(19) United States
(12) Patent Application Publication
(10) Pub. No.: US 2026/0223524 A1
(43) Pub. Date: Jul. 30, 2026

(54) POLYCYCLIC COMPOUND, LIGHT-EMITTING DEVICE AND ELECTRONIC DEVICE

(71) Applicant: Samsung Display Co., LTD., Yongin-si (KR)

(72) Inventors: Se Ran KIM, Yongin-si (KR); Hyeon Deuk KIM, Yongin-si (KR); Hoi Lim KIM, Yongin-si (KR); Byoung Hee PARK, Yongin-si (KR); Seung In BAEK, Yongin-si (KR); Myung Ju SHIN, Yongin-si (KR); Dong Sun YOO, Yongin-si (KR); Jin Kyong HAM, Yongin-si (KR)

(21) Appl. No.: 19/347,019

(22) Filed: Oct. 1, 2025

(30) Foreign Application Priority Data
Jan. 24, 2025 (KR) 10-2025-0011628

(51) Int. Cl. H10K 50/11; C07C 13/553; H10K 50/15; H10K 50/16; H10K 85/30; H10K 85/60; H10K 101/30; H10K 102/00

(52) U.S. Cl. CPC H10K 50/11; C07C 13/553; H10K 50/15; H10K 50/16; H10K 85/346; H10K 85/626; H10K 85/633; H10K 85/636; H10K 85/654; H10K 85/6572; H10K 85/6574; H10K 2101/30; H10K 2102/361

(57) ABSTRACT

A polycyclic compound represented by Chemical Formula 1 or 2, a light-emitting device that includes the polycyclic compound, and an electronic device that includes the light-emitting device

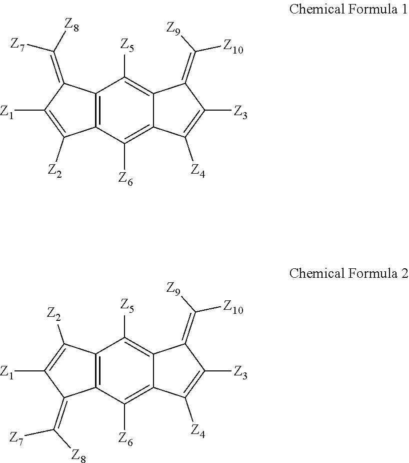

wherein the variables are as described herein,

### CROSS-REFERENCE TO RELATED APPLICATION

This application claims priority to and the benefit Korean Patent Application No. 10-2025-0011628 filed on Jan. 24, 2025 in the Korean Intellectual Property Office (KIPO), and all the benefits accruing therefrom under 35 U.S.C. § 119, the content of which is incorporated herein in its entirety by reference.

### TECHNICAL FIELD

Embodiments of the present application relate to a polycyclic compound, a light-emitting device including the condensed polycyclic compound, and an electronic device including the condensed polycyclic compound.

### BACKGROUND

An organic light-emitting diode (OLED) display has a self-luminous property, and may allow for an improved viewing angle and contrast properties. Additionally, an OLED display may provide a high response speed and a high luminance.

The OLED display may include an emission layer disposed between a first electrode and a second electrode. A hole transferred from the first electrode and an electron transferred from the second electrode may be recombined in the emission layer to generate an exciton. Light emission properties are implemented as the exciton is shifted from an excited state to a ground state.

### SUMMARY

An aspect of the present disclosure includes a polycyclic compound providing improved driving voltage, luminous efficiency and life-span property.

An aspect of the present disclosure includes a light-emitting device having improved driving voltage, luminous efficiency and life-span property.

An aspect of the present disclosure includes an electronic device having improved driving voltage, luminous efficiency, and life-span property.

A polycyclic compound according to the present disclosure may be represented by Chemical Formula 1 or Chemical Formula 2.

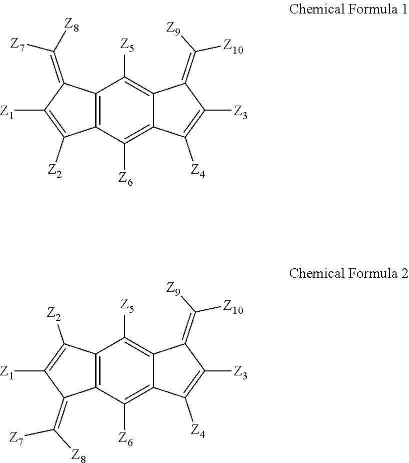

In Chemical Formulae 1 and 2, Z₁ to Z₁₀ are each independently hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ arylalkyl group, a substituted or unsubstituted C₇-C₆₀ alkyl aryl group, a substituted or unsubstituted C₂-C₆₀ heteroaryl group, a substituted or unsubstituted C₂-C₆₀ alkyl heteroaryl group, a substituted or unsubstituted C₃-C₆₀ heteroarylalkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, or a substituted or unsubstituted silyl group, and two or more adjacent groups among Z₁ to Z₁₀ are optionally combined to form a saturated ring or an unsaturated ring.

At least one of Z₁ to Z₁₀ is a substituted or unsubstituted biphenyl group, a substituted or unsubstituted terphenyl group, a substituted or unsubstituted dibenzofuran group, a substituted or unsubstituted dibenzothiophene group, or a substituted or unsubstituted carbazole group, and at least one of a remainder of Z₁ to Z₁₀ is an electron withdrawing group or a group substituted with an electron withdrawing group.

The electron-withdrawing group is a group having a para Hammett substituent constant (σp) of a Hammett rule greater than 0.

A light-emitting device according to the present disclosure may include a first electrode, a second electrode, and an intermediate disposed between the first electrode and the second electrode. The intermediate layer may include at least one light-emitting structure that may include a hole transfer region, an emission layer and an electron transfer region. The hole transfer region may include the above-described polycyclic compound.

In some embodiments, the intermediate layer may include two or more of the light-emitting structures, and at least one of the hole transfer region may include the polycyclic compound.

In some embodiments, the hole transfer region may include a hole injection layer, and the hole injection layer may include the polycyclic compound.

An electronic device according to the present disclosure may include the light-emitting device.

The polycyclic compound, according to some embodiments of the present disclosure, may include an improved driving voltage, luminous efficiency, and life-span property.

The light-emitting device and the electronic device, according to some embodiments of the present disclosure, may have an improved driving voltage, luminous efficiency, and life-span property.

### BRIEF DESCRIPTION OF THE DRAWINGS

FIGS. 1 to 6 are schematic cross-sectional views illustrating light-emitting devices in accordance with example embodiments.

FIG. 7 is a schematic cross-sectional view illustrating a display device in accordance with example embodiments.

FIG. 8 is a schematic cross-sectional view illustrating a display device in accordance with example embodiments.

FIG. 9 is a schematic cross-sectional view illustrating a stack construction of light-emitting structure in a display device in accordance with example embodiments.

FIG. 10 is a schematic cross-sectional view illustrating a display device in accordance with example embodiments.

FIG. 11 is a schematic cross-sectional view illustrating a display device in accordance with example embodiments.

FIG. 12 is a schematic exploded perspective view illustrating an electronic device in accordance with example embodiments.

FIG. 13 is a schematic view illustrating an electronic device in accordance with example embodiments.

FIG. 14 is a block diagram of an electronic device in accordance with an embodiment.

FIG. 15 shows schematic diagrams of electronic devices in accordance with various embodiments.

### DETAILED DESCRIPTION OF THE EMBODIMENTS

A polycyclic compound according to embodiments of the present disclosure may include a group including two or more benzene rings capable of protecting a core, and thus interaction with surrounding compounds may be suppressed or prevented. For example, the polycyclic compound may suppress formation of by-products such as charge transfer complexes due to unnecessary reactions with the surrounding compounds. Thus, consumption of the polycyclic compound by the by-products may be prevented, and the polycyclic compound may have improved stability.

Accordingly, the polycyclic compound may have a deep lowest unoccupied molecular orbit (LUMO) energy level, high hole mobility, low electron mobility, and high glass transition temperature.

According to the present disclosure, a light-emitting device and an electronic device including the polycyclic compound are provided. The light-emitting device and the electronic device may have high luminous efficiency and improved life-span properties at a low driving voltage.

### Definitions of Terminology

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third, or the like may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, ±10%, or ±5% of the stated value.

In the present specification, the term “substituted or unsubstituted” may refer to being substituted or unsubstituted by one or more substituent such as, e.g., a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group (e.g., a C₁-C₆₀, C₁-C₁₀ alkyl group), an alkenyl group (e.g., a C₂-C₆₀, C₂-C₁₀ alkenyl group), an alkynyl group (e.g., a C₂-C₆₀, C₂-C₁₀ alkynyl group), an alkoxy group (e.g., a C₁-C₆₀, C₁-C₁₀ alkoxy group), a hydrocarbon ring group, an aryl group (e.g., a C₆-C₆₀ aryl group), or a heterocyclic group (e.g., a C₁-C₆₀ heterocyclic group). For example, the term “substituted alkyl group” may refer to a group in which at least one of hydrogen atoms of the alkyl group is substituted with the above-described substituent, and thus the substituent is further bonded to a carbon atom of the alkyl group.

The substituent may include a combination of substituents of the groups described above. For example, at least one hydrogen atom in the alkyl group, the aryl group, etc., included as a substituent may be substituted with a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group, an alkenyl group, an alkynyl group, a hydrocarbon ring group, an aryl group, a heterocyclic group, or a combination thereof.

In the substituents described above, a multivalent substituent such as an amino group, a phosphine sulfide group, a phosphine oxide group, a sulfinyl group, a sulfonyl group, an oxy group, a carbonyl group, an ester group, etc., may each independently be substituted with a C₁-C₁₀ alkyl group, a C₁-C₁₀ alkenyl group, a C₁-C₁₀ alkynyl group, a C₆-C₁₀ aryl group a C₆-C₁₀ alkyl aryl group, or C₆-C₁₀ aryl alkyl group.

In the specification, the term “substituted or unsubstituted Cₐ-C_{b} Y group” the range of a to b refers to the number of carbon atoms in an unsubstituted Y group, and may not include the number of carbon atoms of a substituent.

In the specification, an alkyl group may be a monovalent hydrocarbon group in which one hydrogen atom is removed from a linear or branched hydrocarbon group. Examples of an alkyl group may include a methyl group, an ethyl group, a propyl group, a sec-butyl group, a tert-butyl group, an iso-butyl group, a pentyl group, a neopentyl group, a 2-ethyl butyl group, a 3,3-dimethyl butyl group, a hexyl group, a heptyl group, an octyl group, etc.

In the specification, an alkylene group may be a divalent hydrocarbon group in which two hydrogen atoms are removed from a linear or branched hydrocarbon group.

In the specification, an alkenyl group may have the same skeleton as that of an alkyl group, and may be a monovalent hydrocarbon group that includes at least one carbon-carbon double bond. In the specification, an alkenylene group may be a divalent hydrocarbon group in which one hydrogen atom is further removed from an alkenyl group.

In the specification, an alkynyl group may have the same skeleton as that of an alkyl group, and may be a monovalent hydrocarbon group that includes at least one carbon-carbon triple bond. In the specification, an alkynylene group may be a divalent hydrocarbon group in which one hydrogen atom is further removed from an alkynyl group.

In the specification, an aryl group may be a monovalent hydrocarbon group in which one hydrogen atom is removed from a hydrocarbon group having an aromatic structure. The definition of an aryl group may also encompass a group in which multiple aromatic rings are directly connected, such as a biphenyl group. Examples of an aryl group may include, e.g., a phenyl group, a naphthyl group, an anthracenyl group, a phenanthrenyl group, a pyrenyl group, a fluorenyl group, a tetracenyl group, a biphenyl group, a terphenyl group, a quaterphenyl group, a chrysenyl group, etc.

In the specification, a group in which two or more aryl rings are condensed to each other or linked to each other by an alicyclic hydrocarbon ring, such as a fluorenyl group, can be encompassed in the definition of an aryl group.

For example, a biphenyl group may be interpreted as an aryl group, or may be interpreted as a phenyl group that is substituted with a phenyl group.

In the specification, an arylene group may be a divalent hydrocarbon group in which two hydrogen atoms are removed from an aryl group.

In the specification, an alkyl aryl group is an aryl group connected to a moiety by an alkyl group, e.g., a benzyl group is an alkyl aryl group. Similarly, an alkyl heteroaryl group is a heteroaryl group connected to a moiety via an alkyl group, e.g. methyl thiophene.

In the specification, a heteroaryl group may be a monovalent group having an aromatic structure that includes at least one heteroatom such as B, O, P, S, Si, Se, and Ge as a ring-forming atom. In the specification, a heteroarylene group may be a divalent group having an aromatic structure that includes at least one heteroatom such as B, O, P, S, Si, Se, and Ge as a ring-forming atom. When a heteroaryl group or a heteroarylene group includes two or more heteroatoms, the two or more heteroatoms may be the same as or different from each other. In the specification, a heterocycloalkenyl group may be a divalent group that includes at least one heteroatom such as B, O, P, S, Si, Se, and Ge, and has double bond. The double bond may be a carbon-carbon double bond or a carbon-heteroatom double bond.

In the specification, an aryloxy group may be —OA₁₀₂ (wherein A₁₀₂ is the C₆-C₆₀ aryl group), and an alkylthio group may be a monovalent group represented by —SA₁₀₁ (wherein A₁₀₁ is the C₁-C₆₀ alkyl group).

In the specification, a group in which two or more aryl rings are condensed or linked to a non-aromatic heterocyclic ring, such as a carbazole group, can also be encompassed in the definition of a heteroaryl group.

In the specification, the term “cyclic group” may encompass a monocyclic group or a polycyclic group, and may also encompass an alicyclic ring or an aromatic ring.

In the specification, the term “polycyclic group” may be a group in which two or more rings are connected to each other or condensed to each other through one or more atoms. For example, a polycyclic structure may include a bicyclic structure through a bridge carbon, a spiro structure, a fused structure, etc.

In the specification, the term “condensed group” or “condensed ring structure” may each be a group in which two or more adjacent rings share two or more atoms among the above-described polycyclic structures. Examples of a condensed ring structure may include naphthalene, anthracene, phenanthrene, fluorene, pyrene, benzopyrene, pentacene, polyacene, helicene, etc.

In the specification, the term “carbocyclic group (e.g., C₃-C₆₀ carbocyclic group)” may be a cyclic group in which carbon atoms are the only ring-forming atoms. In the specification, a heterocyclic group (e.g., a C₁-C₆₀ heterocyclic group) may be a cyclic group that includes at least one heteroatom as a ring-forming atom, in addition to carbon atoms.

In the specification, a carbocyclic group and a heterocyclic group may each independently be a monocyclic group that consists of one ring or a polycyclic group in which two or more rings are condensed.

### Polycyclic Compound

A polycyclic compound according to embodiments may be represented by Chemical Formula 1 or Chemical Formula 2 below.

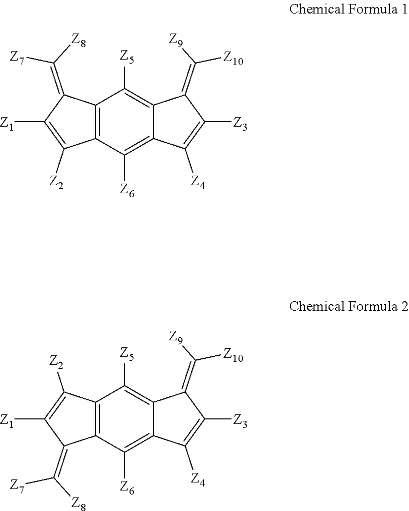

In Chemical Formulae 1 and 2, Z₁ to Z₁₀ may each independently be hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ arylalkyl group, a substituted or unsubstituted C₇-C₆₀ alkyl aryl group, a substituted or unsubstituted C₂-C₆₀ heteroaryl group, a substituted or unsubstituted C₃-C₆₀ alkyl heteroaryl group, a substituted or unsubstituted C₃-C₆₀ heteroarylalkyl group, a substituted or unsubstituted C₆-C₆₀ aryloxy group, a substituted or unsubstituted C₆-C₆₀ arylthio group, a substituted or unsubstituted C₅-C₆₀ condensed polycyclic group, or a substituted or unsubstituted silyl group.

In some embodiments, two or more adjacent groups among Z₁ to Z₁₀ may optionally be combined to form a saturated ring or an unsaturated ring.

At least one of Z₁ to Z₁₀ may be a substituted or unsubstituted biphenyl group, a substituted or unsubstituted terphenyl group, a substituted or unsubstituted dibenzofuran group, a substituted or unsubstituted dibenzothiophene group, or a substituted or unsubstituted carbazole group, and at least one of a remainder of Z₁ to Z₁₀ may be an electron withdrawing group or a group substituted with an electron withdrawing group.

The electron-withdrawing group may be a group having a para Hammett substituent constant (σp) of a Hammett rule greater than 0.

The polycyclic compound may improve hole mobility and reduce electron mobility to generate excitons with high efficiency, and may have a high glass transition temperature so that deterioration may be prevented or suppressed at high temperature.

In an embodiment, the saturated ring may each be independently a 5-membered ring, a 6-membered ring, or a 7-membered ring. The saturated ring may be unsubstituted or substituted with at least one of deuterium, —F, —Cl, —CD₃, —CD₂H, —CDH₂, a C₁-C₁₀ straight-chain alkyl group, a C₃-C₁₀ branched alkyl group, a C₂-C₁₀ straight-chain alkenyl group, a C₃-C₁₀ branched alkenyl group, or a C₆-C₁₀ aryl group. For example, the saturated ring may be a ring having 2 to 60 carbon atoms, and may be selected from a hydrocarbon ring, a heteroatom-containing ring, or a combination thereof.

In some embodiments, the unsaturated rings may be a 5-membered ring, a 6-membered ring, or a 7-membered ring. The unsaturated ring may be, e.g., a cycloalkene or an aromatic ring including a C═C unsaturated double bond. For example, the unsaturated ring may be a ring having 2 to 60 carbon atoms, and may be selected from a hydrocarbon ring, heteroatom-containing ring, or a combination thereof.

The unsaturated ring and the saturated ring may each independently be unsubstituted or substituted with at least one of deuterium, —F, —Cl, —CD₃, —CD₂H, —CDH₂, a C₁-C₁₀ straight-chain alkyl group, a C₃-C₁₀ branched alkyl group, a C₂-C₁₀ straight-chain alkenyl group, a C₃-C₁₀ branched alkenyl group, and a C₆-C₁₀ aryl group.

In some embodiments, the substituted or unsubstituted C₅-C₆₀ condensed polycyclic group may be a condensed polycyclic group in which a C₄-C₁₀ aliphatic hydrocarbon ring and a C₆-C₅₀ aromatic hydrocarbon ring are condensed. The condensed polycyclic group may have a structure in which, e.g., one C₄-C₆ aliphatic hydrocarbon ring is condensed between two C₆-C₁₅ aromatic hydrocarbon rings. The condensed polycyclic group may be, e.g., a substituted or unsubstituted carbazole group, a substituted or unsubstituted fluorene group, or a spiro-bifluorene group.

In some embodiment, e.g., the silyl group may be —Si(R^{Sa})(R^{Sb})(R^{Sc}), and R^{Sa}, R^{Sb}, and R^{Sc} may each independently be hydrogen, deuterium, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₂-C₆₀ heteroaryl group, a substituted or unsubstituted C₃-C₆₀ alkyl heteroaryl group, a substituted or unsubstituted C₃-C₆₀ heteroarylalkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group. The condensed polycyclic group may be defined as described above.

The Hammett rule may be used as a measure of a degree of electron-withdrawing or electron-donating of a functional group. The substituent constant (ap) determined by the Hammett rule can be found in literature such as “A survey of Hammett substituent constants and resonance and field parameters,” but even without reference to such literature, the substituent constant (ap) can be measured based on the Hammett rule as shown in Equation 1 (EQ1):

$$log \frac{K}{{K}_{o}} = σ ρ \qquad ( EQ1 )$$

In Equation 1, K is the equilibrium constant given for an equilibrium reaction with substituent R and the reference constant Kₒ when R is a hydrogen atom to the substituent constant σ, which depends only on the specific substituent R and the reaction rate constant p, which depends on the type of the reaction, but not the substituent used.

In some embodiments, the electron-withdrawing may have a substituent constant σp of the Hammett rule greater than 0.05.

In some embodiments, at least one of Z₁, Z₃, Z₅ and Z₆ in Chemical Formulae 1 and 2 may each independently be a group represented by Chemical Formulae 3-1 to 3-4 below.

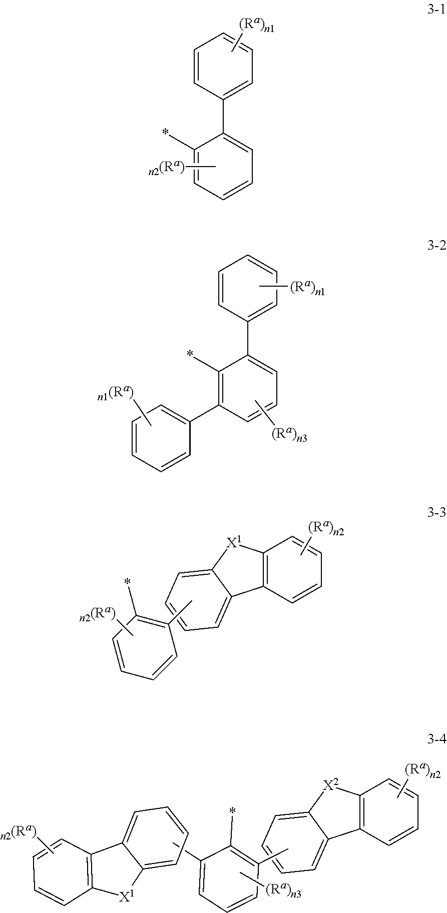

In Chemical Formulae 3-1 to 3-4, R^{a} may be hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a substituted or unsubstituted C₁-C₃₀ alkylthio group, a substituted or unsubstituted C₃-C₃₀ cycloalkyl group, a substituted or unsubstituted C₅-C₃₀ cycloalkenyl group, a substituted or unsubstituted C₁-C₃₀ heterocycloalkyl group, a substituted or unsubstituted C₁-C₃₀ heterocycloalkenyl group, a substituted or unsubstituted C₆-C₃₀ aryl group, a substituted or unsubstituted C₇-C₃₀ arylalkyl group, a substituted or unsubstituted C₇-C₆₀ alkyl aryl group, a substituted or unsubstituted C₂-C₃₀ heteroaryl group, a substituted or unsubstituted C₃-C₆₀ alkyl heteroaryl group, a substituted or unsubstituted C₃-C₃₀ heteroarylalkyl group, a substituted or unsubstituted C₆-C₃₀ aryloxy group, or a substituted or unsubstituted C₆-C₃₀ arylthio group.

When a plurality of R^{a} is included, the plurality of R^{a} may be the same as or different from each other. Two or more adjacent groups among the plurality of R^{a} may optionally be combined to form a saturated ring or an unsaturated ring.

n1 is an integer from 0 to 5, n2 is an integer from 0 to 4, and n3 is an integer from 0 to 3.

X¹ and X² may each independently be CR₁R₂, NR₃, O, S, or Se.

R₁ to R₃ may each independently be hydrogen, deuterium, —CD₃, —CD₂H, —CDH₂, —CF₃, —CF₂H, —CFH₂, a substituted or unsubstituted C₁-C₃₀ alkyl group, a substituted or unsubstituted C₆-C₃₀ aryl group, a substituted or unsubstituted C₇-C₃₀ arylalkyl group, a substituted or unsubstituted C₇-C₃₀ alkyl aryl group, a substituted or unsubstituted C₂-C₃₀ heteroaryl group, a substituted or unsubstituted C₃-C₃₀ alkyl heteroaryl group, or a substituted or unsubstituted C₃-C₃₀ heteroarylalkyl group. *- represents a bonding position.

In some embodiments, Z₁ and Z₃ may each independently be a group represented by Chemical Formulae 3-1 to 3-4, or Z₅ and Z₆ may each independently be a group represented by Chemical Formulae 3-1 to 3-4. Accordingly, an electron distribution region of the core of the polycyclic compound may be protected, so that unnecessary side reactions with surrounding compounds may be suppressed or prevented.

In some embodiments, the electron withdrawing group may be —F, —Cl, —Br, —I, —SF₅, —CF₃, —CN, —SCN, —SOCH₃, —SOCH₂CH₃, —SCH(CH₃)₂, —NO₂, or a π electron-depleted nitrogen-containing C₃-C₃₀ cyclic group. Accordingly, HOMO and LUMO energy levels of the polycyclic compound may be formed at a required level to further enhance hole mobility and further reduce electron mobility.

In some embodiments, the electron withdrawing group may be —F, —Cl, —Br, —I, —SF₅, —CF₃, —CN, —SCN, —SOCH₃, —SOCH₂CH₃, —SCH(CH₃)₂, —NO₂, a substituted or unsubstituted triazine group, a substituted or unsubstituted thiazole group, a substituted or unsubstituted benzothiazole group, a substituted or unsubstituted pyrazine group, a substituted or unsubstituted pyridine group, a substituted or unsubstituted pyridazine group, a substituted or unsubstituted pyrimidine group, a substituted or unsubstituted naphthyridine group, a substituted or unsubstituted phthalazine group, a substituted or unsubstituted quinoline group, a substituted or unsubstituted quinazoline group, a substituted or unsubstituted benzocinnoline group, a substituted or unsubstituted phenanthroline group, or a substituted or unsubstituted acridine group.

Accordingly, hole mobility of the polycyclic compound may be further improved. In some embodiments, the polycyclic compound may be represented by any one of Chemical Formulae 1-1 to 1-10.

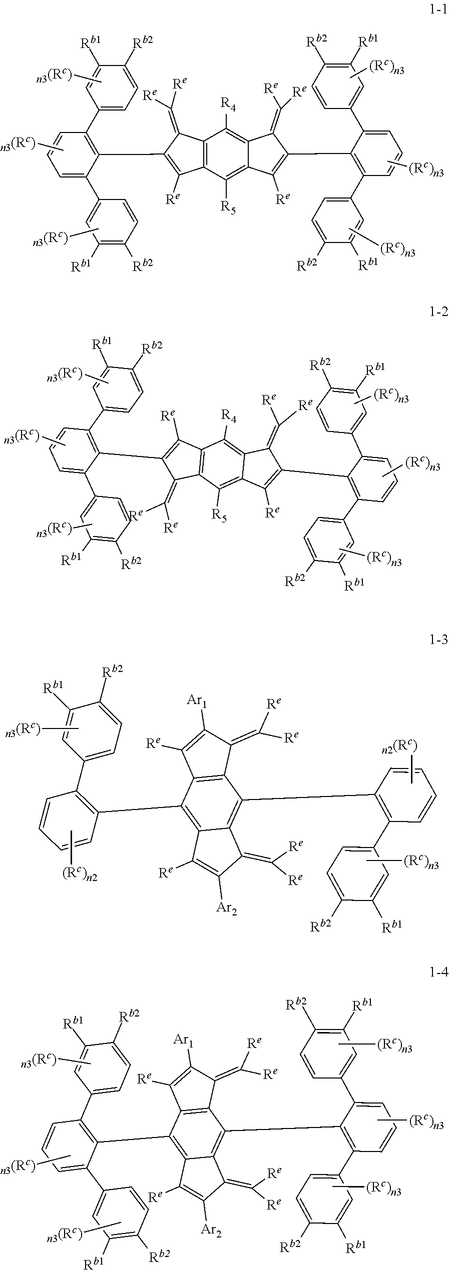

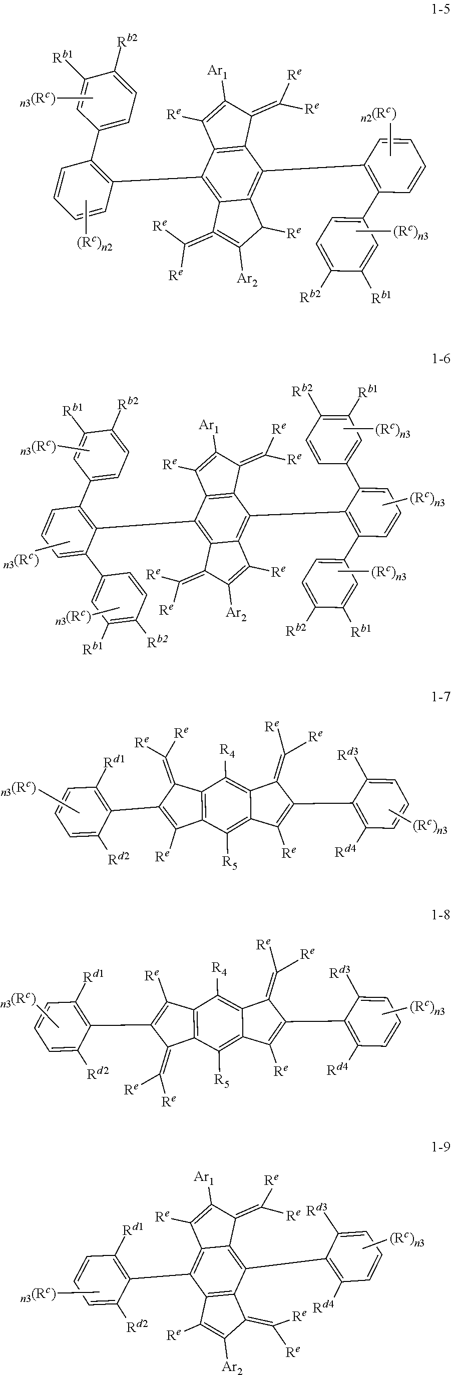

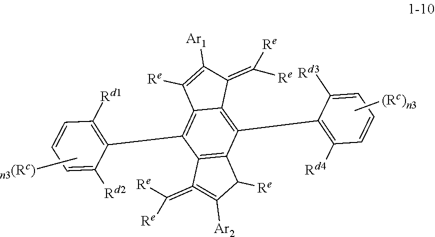

In Chemical Formulas 1-1 to 1-10, n2 may be an integer from 0 to 4, and n3 may be an integer from 0 to 3. When n2 and n3 are 2 or more, multiple R^{c} may be the same as or different from each other.

R^{c} may be hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₃₀ alkyl group, a substituted or unsubstituted C₃-C₃₀ cycloalkyl group, a substituted or unsubstituted C₁-C₃₀ heterocycloalkyl group, a substituted or unsubstituted C₆-C₃₀ aryl group, a substituted or unsubstituted C₇-C₃₀ arylalkyl group, a substituted or unsubstituted C₇-C₃₀ alkyl aryl group, a substituted or unsubstituted C₂-C₃₀ heteroaryl group, a substituted or unsubstituted C₃-C₃₀ alkyl heteroaryl group, or a substituted or unsubstituted C₃-C₃₀ heteroarylalkyl group. When a plurality of R^{c} is included, two or more adjacent groups among the plurality of R^{c} may optionally be combined to form a saturated ring or an unsaturated ring.

A plurality of each of R^{b1} and R^{b2} may be the same as or different from each other, and may each independently be hydrogen, deuterium, —CN, —F, —CD₃, —CD₂H, —CDH₂, —CF₃, —CF₂H, —CFH₂, a substituted or unsubstituted C₆-C₃₀ aryl group, or a substituted or unsubstituted C₂-C₃₀ heteroaryl group.

Ar₁ and Ar₂ may each independently be a substituted or unsubstituted C₆-C₃₀ aryl group, or a substituted or unsubstituted C₂-C₃₀ heteroaryl group.

Two or more R^{e} may be the same as or different from each other, and may each independently be the above-mentioned electron withdrawing groups.

R₄ and R₅ may each independently be hydrogen, deuterium, —CD₃, —CD₂H, —CDH₂, —CF₃, —CF₂H, —CFH₂, a substituted or unsubstituted C₁-C₃₀ alkyl group, or the above-mentioned electron withdrawing groups.

R^{d1} to R^{d4} may each independently be the electron withdrawing group, a substituted or unsubstituted dibenzofuran group, a substituted or unsubstituted dibenzothiophene group, or a substituted or unsubstituted carbazole group. Two or more of R^{d1} to R^{d4} may each independently be a substituted or unsubstituted dibenzofuran group, a substituted or unsubstituted dibenzothiophene group, or a substituted or unsubstituted carbazole group.

Accordingly, the LUMO energy level of the polycyclic compound may become deep, hole mobility may be improved, and high-temperature stability may also be improved.

In some embodiments, in Chemical Formulae 1-1 to 1-6, multiple R^{b1} may each independently a substituted or unsubstituted C₆-C₃₀ aryl group, or a substituted or unsubstituted C₂-C₃₀ heteroaryl group, and multiple R^{b2} may each independently be hydrogen, deuterium, —CN, —F, —CD₃, —CD₂H, —CDH₂, —CF₃, —CF₂H, or —CFH₂.

In some embodiments, multiple R^{b1} may be a substituted or unsubstituted C₆-C₃₀ aryl group, and the aryl group may include one or more electron withdrawing group.

R^{b1} may be bonded to another arylene group bonded to a phenylene group at a meta position relative to the bonded phenylene group. Thus, a core protecting effect of the polycyclic compound may be improved by a rotation through a single bond compared to the case being boned to a para position.

In some embodiments, in Chemical Formulae 1-1 and 1-7, R₄ may be the electron withdrawing group, and R₅ may each independently be hydrogen, deuterium, —CD₃, —CD₂H, —CDH₂, or a substituted or unsubstituted C₁-C₃₀ alkyl group. Accordingly, polarity of the polycyclic compound may be increased, thereby further improving hole injection properties.

In some embodiments, in Chemical Formulae 1-7 to 1-10, at least one of R^{d1} and R^{d2}, and at least one of R^{d3} and R^{d4} may each independently be a substituted or unsubstituted dibenzofuran group, a substituted or unsubstituted dibenzothiophene group, or a substituted or unsubstituted carbazole group.

In some embodiments, a degree of deuteration of the polycyclic compound according to Equation 1 may be in a range from 10% to 100%:

$$Degree of Deuteration ( \% ) = ( the number of deuterium atoms ) / ( the number of deuterium atoms + the number of hydrogen atoms ) \times 100 \qquad [ Equation 1 ]$$

In Equation 1, the number of deuterium atoms and the number of hydrogen atoms refer to the numbers of deuterium and hydrogen atoms, respectively, included in the polycyclic compound. The degree of deuteration may be, e.g., 50% or more, 70% or more, 80% or more, or 90% or more. As the degree of deuteration becomes higher, high temperature stability may be enhanced.

In some embodiments, the polycyclic compound may have a hole mobility in a range from 2.5×10⁻⁴ square centimeters per Volt-second (cm²/Vs) to 1.0×10⁻³ cm²/Vs. For example, the hole mobility may be, for example, 3.0×10⁻⁴ cm²/Vs or more.

In some embodiments, the polycyclic compound may have an electron mobility in a range from 8.0×10⁻⁴ cm²/Vs to 2.5×10⁻³ cm²/Vs. The electron mobility may be, e.g., 2.0×10⁻³ cm²/Vs or less.

Accordingly, the hole injection properties of the polycyclic compound may be further improved.

In some embodiments, the polycyclic compound may have a glass transition temperature of 125° C. or more. Accordingly, high-temperature stability may be enhanced, and deterioration during a high-temperature manufacturing process may be suppressed or reduced.

In some embodiments, the polycyclic compound may have a LUMO energy level in a range from −5.0 electronVolt (eV) to −6.0 eV.

Accordingly, driving voltage and life-span properties of a light-emitting device to which the polycyclic compound is applied may be further improved.

In some embodiments, the polycyclic compound may be any one of compounds 1 to 93 below.

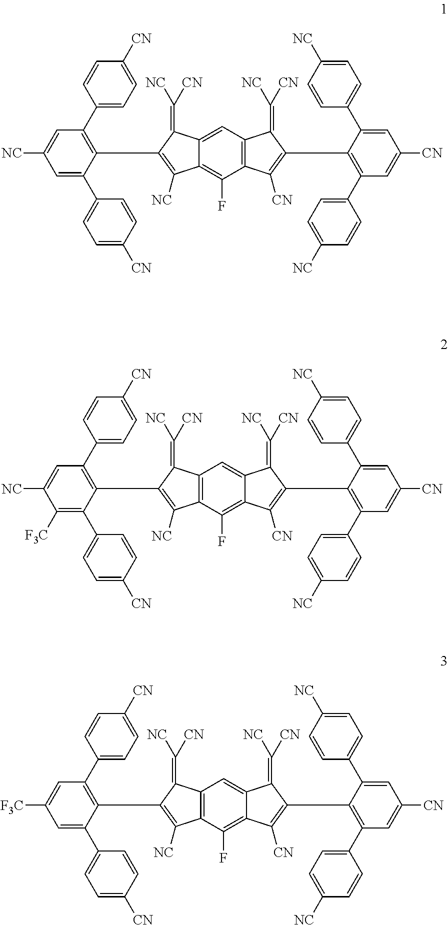 [SMILES: N#CC(C#N)=C1C(c2c(-c3ccc(C#N)cc3)cc(C#N)cc2-c2ccc(C#N)cc2)=C(C#N)c2c1cc1c(c2F)C(C#N)=C(c2c(-c3ccc(C#N)cc3)cc(C#N)cc2-c2ccc(C#N)cc2)C1=C(C#N)C#N ; N#CC(C#N)=C1C(c2c(-c3ccc(C#N)cc3)cc(C#N)cc2-c2ccc(C#N)cc2)=C(C#N)c2c1cc1c(c2F)C(C#N)=C(c2c(-c3ccc(C#N)cc3)cc(C#N)c(C(F)(F)F)c2-c2ccc(C#N)cc2)C1=C(C#N)C#N ; N#CC(C#N)=C1C(c2c(-c3ccc(C#N)cc3)cc(C#N)cc2-c2ccc(C#N)cc2)=C(C#N)c2c1cc1c(c2F)C(C#N)=C(c2c(-c3ccc(C#N)cc3)cc(C(F)(F)F)cc2-c2ccc(C#N)cc2)C1=C(C#N)C#N]

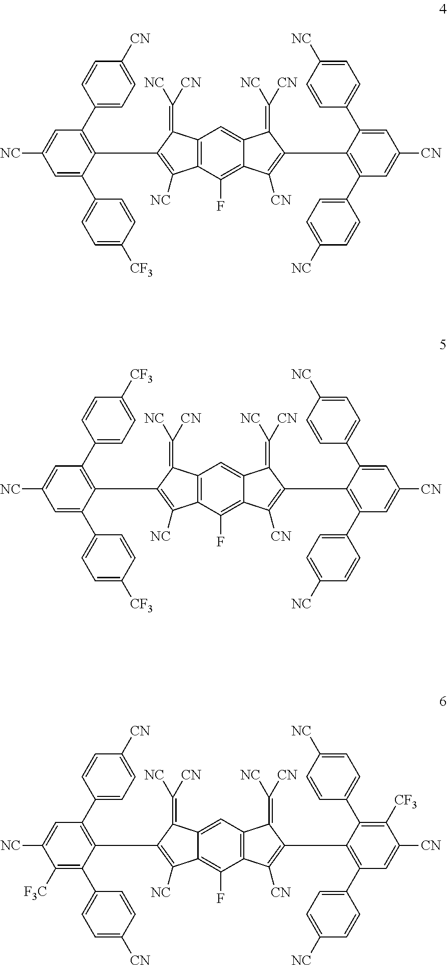 [SMILES: N#CC(C#N)=C1C(c2c(-c3ccc(C#N)cc3)cc(C#N)cc2-c2ccc(C#N)cc2)=C(C#N)c2c1cc1c(c2F)C(C#N)=C(c2c(-c3ccc(C#N)cc3)cc(C#N)cc2-c2ccc(C(F)(F)F)cc2)C1=C(C#N)C#N ; N#CC(C#N)=C1C(c2c(-c3ccc(C#N)cc3)cc(C#N)cc2-c2ccc(C#N)cc2)=C(C#N)c2c1cc1c(c2F)C(C#N)=C(c2c(-c3ccc(C(F)(F)F)cc3)cc(C#N)cc2-c2ccc(C(F)(F)F)cc2)C1=C(C#N)C#N ; N#CC(C#N)=C1C(c2c(-c3ccc(C#N)cc3)cc(C#N)c(C(F)(F)F)c2-c2ccc(C#N)cc2)=C(C#N)c2c1cc1c(c2F)C(C#N)=C(c2c(-c3ccc(C#N)cc3)cc(C#N)c(C(F)(F)F)c2-c2ccc(C#N)cc2)C1=C(C#N)C#N]

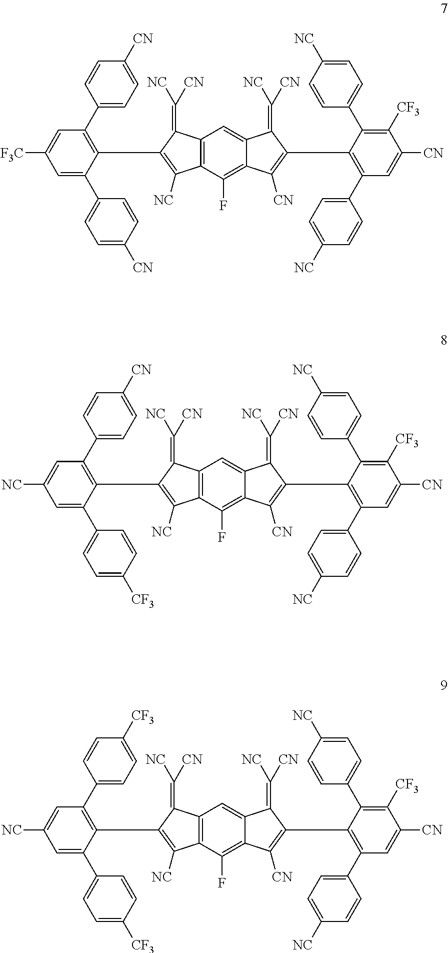 [SMILES: N#CC(C#N)=C1C(c2c(-c3ccc(C#N)cc3)cc(C(F)(F)F)cc2-c2ccc(C#N)cc2)=C(C#N)c2c1cc1c(c2F)C(C#N)=C(c2c(-c3ccc(C#N)cc3)cc(C#N)c(C(F)(F)F)c2-c2ccc(C#N)cc2)C1=C(C#N)C#N ; N#CC(C#N)=C1C(c2c(-c3ccc(C#N)cc3)cc(C#N)cc2-c2ccc(C(F)(F)F)cc2)=C(C#N)c2c1cc1c(c2F)C(C#N)=C(c2c(-c3ccc(C#N)cc3)cc(C#N)c(C(F)(F)F)c2-c2ccc(C#N)cc2)C1=C(C#N)C#N ; N#CC(C#N)=C1C(c2c(-c3ccc(C(F)(F)F)cc3)cc(C#N)cc2-c2ccc(C(F)(F)F)cc2)=C(C#N)c2c1cc1c(c2F)C(C#N)=C(c2c(-c3ccc(C#N)cc3)cc(C#N)c(C(F)(F)F)c2-c2ccc(C#N)cc2)C1=C(C#N)C#N]

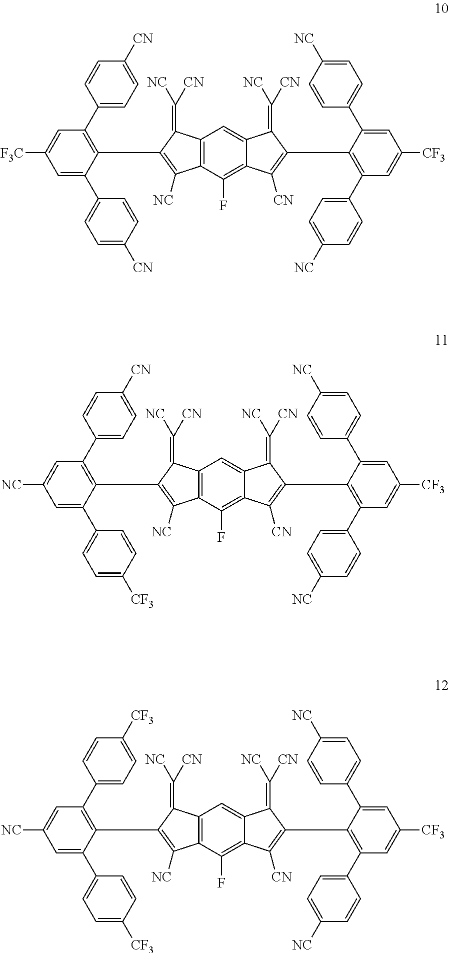 [SMILES: N#CC(C#N)=C1C(c2c(-c3ccc(C#N)cc3)cc(C(F)(F)F)cc2-c2ccc(C#N)cc2)=C(C#N)c2c1cc1c(c2F)C(C#N)=C(c2c(-c3ccc(C#N)cc3)cc(C(F)(F)F)cc2-c2ccc(C#N)cc2)C1=C(C#N)C#N ; N#CC(C#N)=C1C(c2c(-c3ccc(C#N)cc3)cc(C#N)cc2-c2ccc(C(F)(F)F)cc2)=C(C#N)c2c1cc1c(c2F)C(C#N)=C(c2c(-c3ccc(C#N)cc3)cc(C(F)(F)F)cc2-c2ccc(C#N)cc2)C1=C(C#N)C#N ; N#CC(C#N)=C1C(c2c(-c3ccc(C(F)(F)F)cc3)cc(C#N)cc2-c2ccc(C(F)(F)F)cc2)=C(C#N)c2c1cc1c(c2F)C(C#N)=C(c2c(-c3ccc(C#N)cc3)cc(C(F)(F)F)cc2-c2ccc(C#N)cc2)C1=C(C#N)C#N]

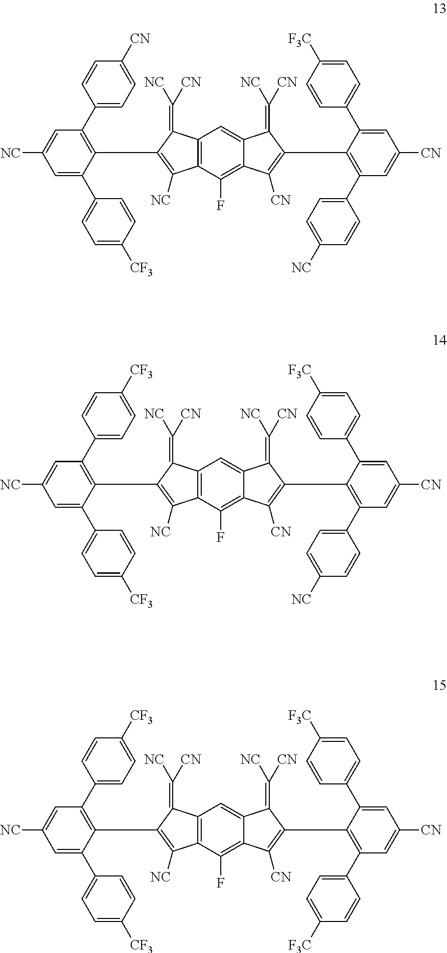 [SMILES: N#CC(C#N)=C1C(c2c(-c3ccc(C#N)cc3)cc(C#N)cc2-c2ccc(C(F)(F)F)cc2)=C(C#N)c2c1cc1c(c2F)C(C#N)=C(c2c(-c3ccc(C#N)cc3)cc(C#N)cc2-c2ccc(C(F)(F)F)cc2)C1=C(C#N)C#N ; N#CC(C#N)=C1C(c2c(-c3ccc(C#N)cc3)cc(C#N)cc2-c2ccc(C(F)(F)F)cc2)=C(C#N)c2c1cc1c(c2F)C(C#N)=C(c2c(-c3ccc(C(F)(F)F)cc3)cc(C#N)cc2-c2ccc(C(F)(F)F)cc2)C1=C(C#N)C#N ; N#CC(C#N)=C1C(c2c(-c3ccc(C(F)(F)F)cc3)cc(C#N)cc2-c2ccc(C(F)(F)F)cc2)=C(C#N)c2c1cc1c(c2F)C(C#N)=C(c2c(-c3ccc(C(F)(F)F)cc3)cc(C#N)cc2-c2ccc(C(F)(F)F)cc2)C1=C(C#N)C#N]

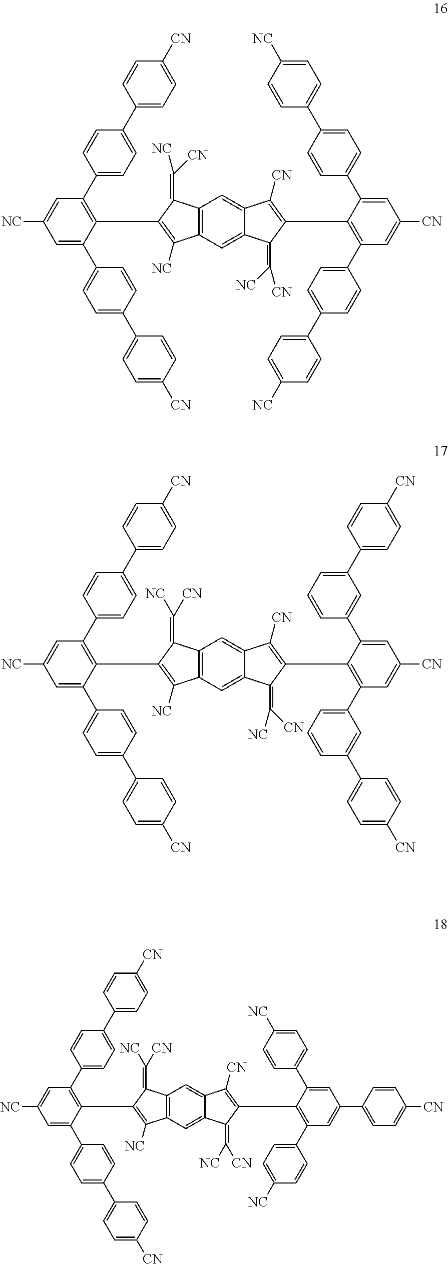

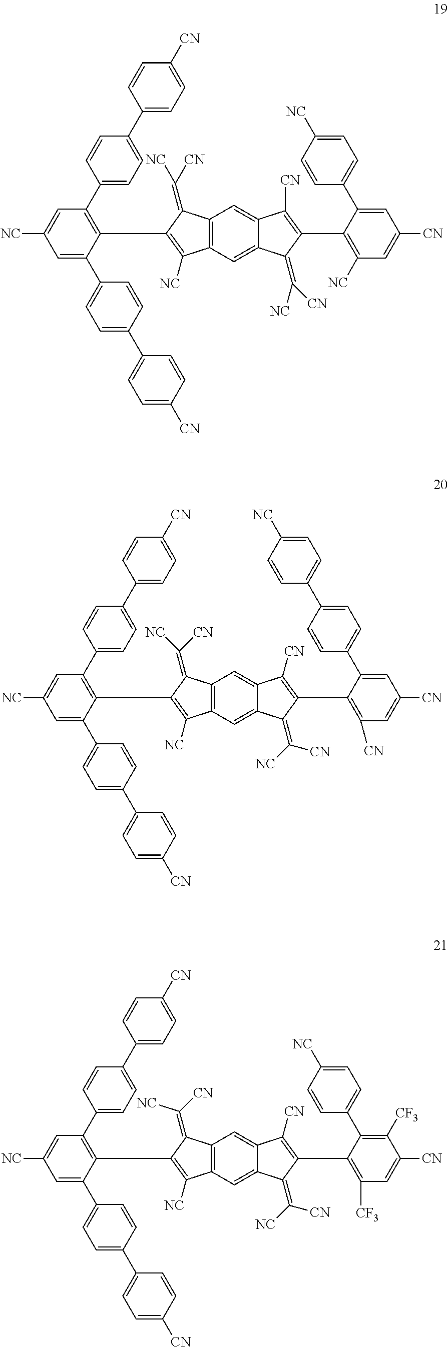 [SMILES: N#CC(C#N)=C1C(c2c(C#N)cc(C#N)cc2-c2ccc(C#N)cc2)=C(C#N)c2cc3c(cc21)C(C#N)=C(c1c(-c2ccc(-c4ccc(C#N)cc4)cc2)cc(C#N)cc1-c1ccc(-c2ccc(C#N)cc2)cc1)C3=C(C#N)C#N ; N#CC(C#N)=C1C(c2c(C#N)cc(C#N)cc2-c2ccc(-c3ccc(C#N)cc3)cc2)=C(C#N)c2cc3c(cc21)C(C#N)=C(c1c(-c2ccc(-c4ccc(C#N)cc4)cc2)cc(C#N)cc1-c1ccc(-c2ccc(C#N)cc2)cc1)C3=C(C#N)C#N ; N#CC(C#N)=C1C(c2c(-c3ccc(-c4ccc(C#N)cc4)cc3)cc(C#N)cc2-c2ccc(-c3ccc(C#N)cc3)cc2)=C(C#N)c2cc3c(cc21)C(C#N)=C(c1c(C(F)(F)F)cc(C#N)c(C(F)(F)F)c1-c1ccc(C#N)cc1)C3=C(C#N)C#N]

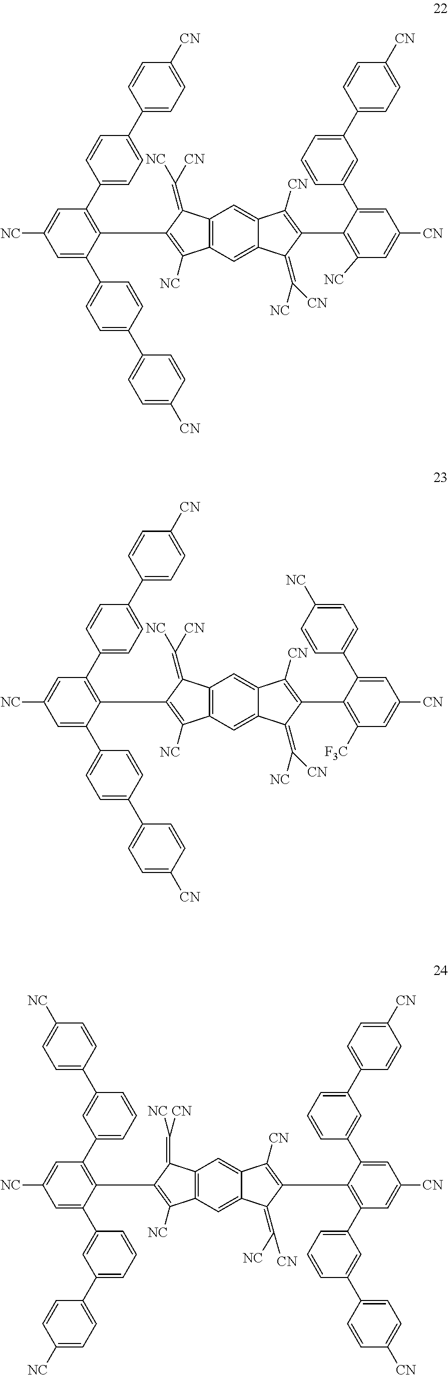

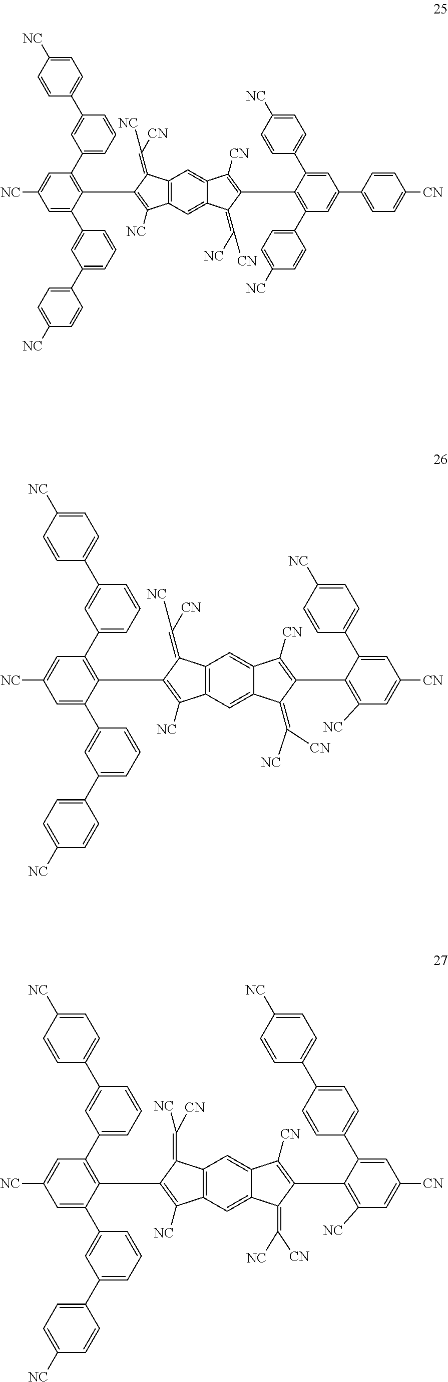 [SMILES: N#CC(C#N)=C1C(c2c(-c3cccc(-c4ccc(C#N)cc4)c3)cc(C#N)cc2-c2cccc(-c3ccc(C#N)cc3)c2)=C(C#N)c2cc3c(cc21)C(C#N)=C(c1c(-c2ccc(C#N)cc2)cc(-c2ccc(C#N)cc2)cc1-c1ccc(C#N)cc1)C3=C(C#N)C#N ; N#CC(C#N)=C1C(c2c(C#N)cc(C#N)cc2-c2ccc(C#N)cc2)=C(C#N)c2cc3c(cc21)C(C#N)=C(c1c(-c2cccc(-c4ccc(C#N)cc4)c2)cc(C#N)cc1-c1cccc(-c2ccc(C#N)cc2)c1)C3=C(C#N)C#N ; N#CC(C#N)=C1C(c2c(C#N)cc(C#N)cc2-c2ccc(-c3ccc(C#N)cc3)cc2)=C(C#N)c2cc3c(cc21)C(C#N)=C(c1c(-c2cccc(-c4ccc(C#N)cc4)c2)cc(C#N)cc1-c1cccc(-c2ccc(C#N)cc2)c1)C3=C(C#N)C#N]

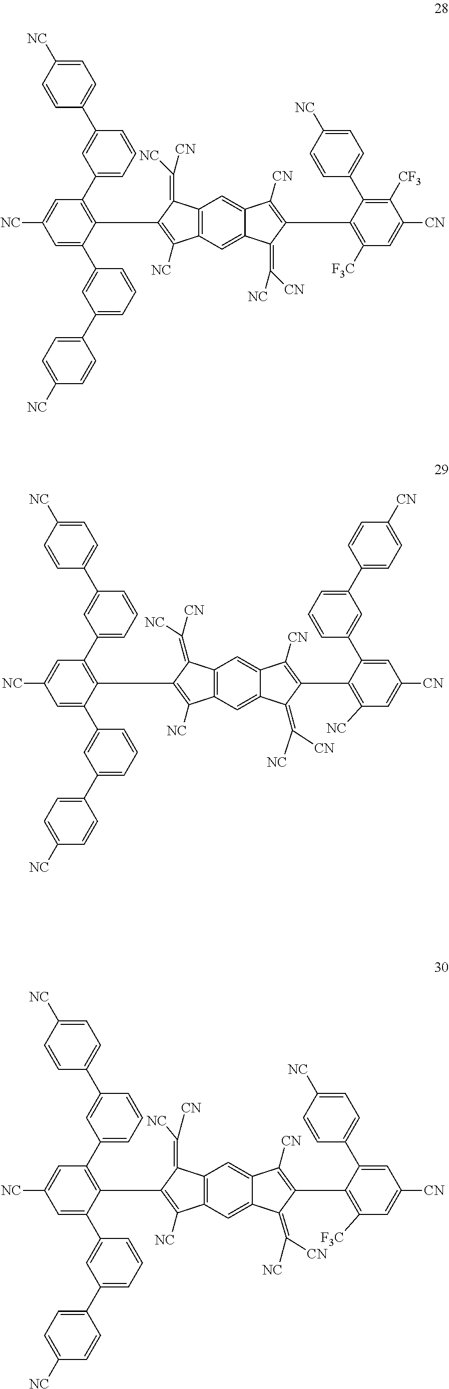 [SMILES: N#CC(C#N)=C1C(c2c(-c3cccc(-c4ccc(C#N)cc4)c3)cc(C#N)cc2-c2cccc(-c3ccc(C#N)cc3)c2)=C(C#N)c2cc3c(cc21)C(C#N)=C(c1c(C(F)(F)F)cc(C#N)c(C(F)(F)F)c1-c1ccc(C#N)cc1)C3=C(C#N)C#N ; N#CC(C#N)=C1C(c2c(C#N)cc(C#N)cc2-c2cccc(-c3ccc(C#N)cc3)c2)=C(C#N)c2cc3c(cc21)C(C#N)=C(c1c(-c2cccc(-c4ccc(C#N)cc4)c2)cc(C#N)cc1-c1cccc(-c2ccc(C#N)cc2)c1)C3=C(C#N)C#N ; N#CC(C#N)=C1C(c2c(-c3cccc(-c4ccc(C#N)cc4)c3)cc(C#N)cc2-c2cccc(-c3ccc(C#N)cc3)c2)=C(C#N)c2cc3c(cc21)C(C#N)=C(c1c(-c2ccc(C#N)cc2)cc(C#N)cc1C(F)(F)F)C3=C(C#N)C#N]

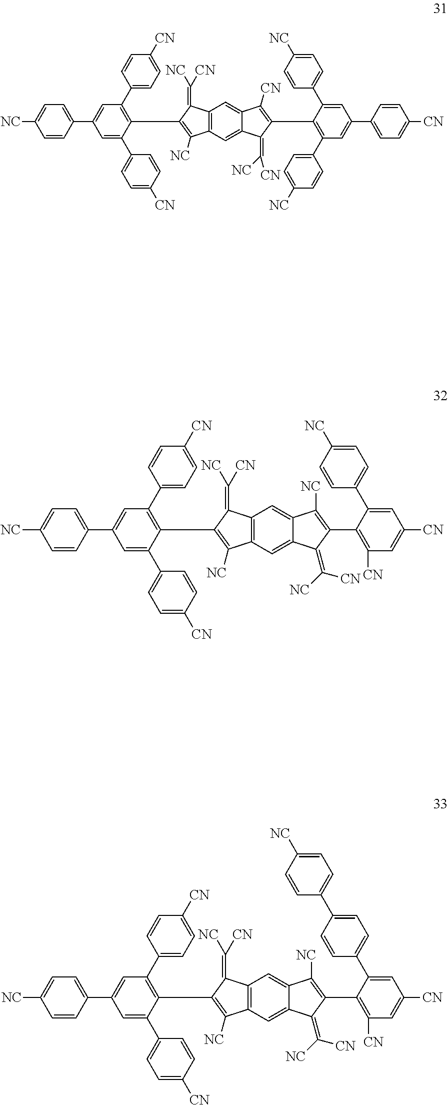 [SMILES: N#CC(C#N)=C1C(c2c(-c3ccc(C#N)cc3)cc(-c3ccc(C#N)cc3)cc2-c2ccc(C#N)cc2)=C(C#N)c2cc3c(cc21)C(C#N)=C(c1c(-c2ccc(C#N)cc2)cc(-c2ccc(C#N)cc2)cc1-c1ccc(C#N)cc1)C3=C(C#N)C#N ; N#CC(C#N)=C1C(c2c(C#N)cc(C#N)cc2-c2ccc(C#N)cc2)=C(C#N)c2cc3c(cc21)C(C#N)=C(c1c(-c2ccc(C#N)cc2)cc(-c2ccc(C#N)cc2)cc1-c1ccc(C#N)cc1)C3=C(C#N)C#N ; N#CC(C#N)=C1C(c2c(C#N)cc(C#N)cc2-c2ccc(-c3ccc(C#N)cc3)cc2)=C(C#N)c2cc3c(cc21)C(C#N)=C(c1c(-c2ccc(C#N)cc2)cc(-c2ccc(C#N)cc2)cc1-c1ccc(C#N)cc1)C3=C(C#N)C#N]

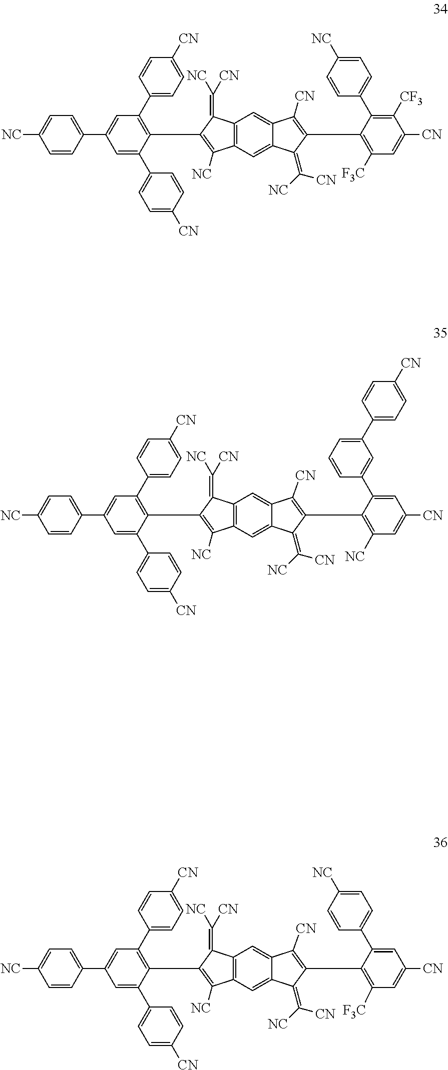 [SMILES: N#CC(C#N)=C1C(c2c(-c3ccc(C#N)cc3)cc(-c3ccc(C#N)cc3)cc2-c2ccc(C#N)cc2)=C(C#N)c2cc3c(cc21)C(C#N)=C(c1c(C(F)(F)F)cc(C#N)c(C(F)(F)F)c1-c1ccc(C#N)cc1)C3=C(C#N)C#N ; N#CC(C#N)=C1C(c2c(C#N)cc(C#N)cc2-c2cccc(-c3ccc(C#N)cc3)c2)=C(C#N)c2cc3c(cc21)C(C#N)=C(c1c(-c2ccc(C#N)cc2)cc(-c2ccc(C#N)cc2)cc1-c1ccc(C#N)cc1)C3=C(C#N)C#N ; N#CC(C#N)=C1C(c2c(-c3ccc(C#N)cc3)cc(-c3ccc(C#N)cc3)cc2-c2ccc(C#N)cc2)=C(C#N)c2cc3c(cc21)C(C#N)=C(c1c(-c2ccc(C#N)cc2)cc(C#N)cc1C(F)(F)F)C3=C(C#N)C#N]

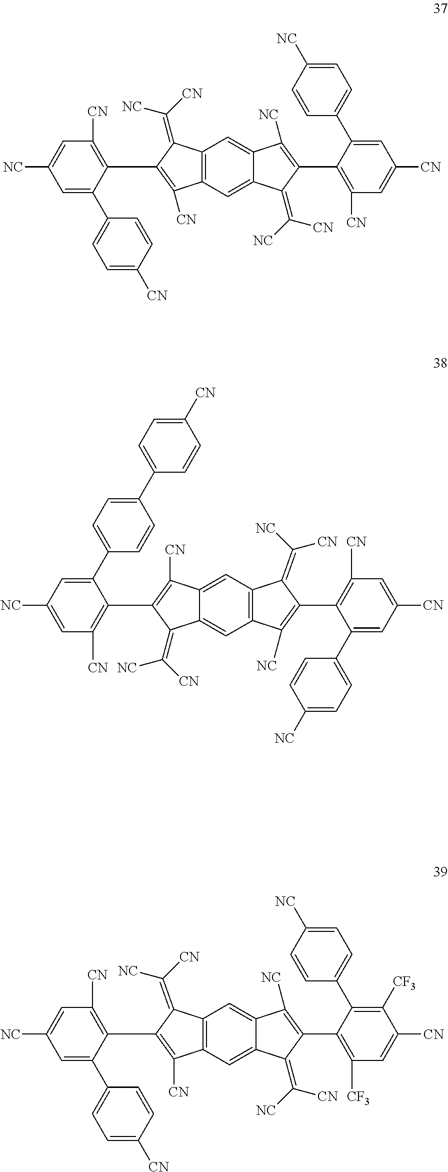 [SMILES: N#CC(C#N)=C1C(c2c(C#N)cc(C#N)cc2-c2ccc(C#N)cc2)=C(C#N)c2cc3c(cc21)C(C#N)=C(c1c(C#N)cc(C#N)cc1-c1ccc(C#N)cc1)C3=C(C#N)C#N ; N#CC(C#N)=C1C(c2c(C#N)cc(C#N)cc2-c2ccc(C#N)cc2)=C(C#N)c2cc3c(cc21)C(C#N)=C(c1c(C#N)cc(C#N)cc1-c1ccc(-c2ccc(C#N)cc2)cc1)C3=C(C#N)C#N ; N#CC(C#N)=C1C(c2c(C#N)cc(C#N)cc2-c2ccc(C#N)cc2)=C(C#N)c2cc3c(cc21)C(C#N)=C(c1c(C(F)(F)F)cc(C#N)c(C(F)(F)F)c1-c1ccc(C#N)cc1)C3=C(C#N)C#N]

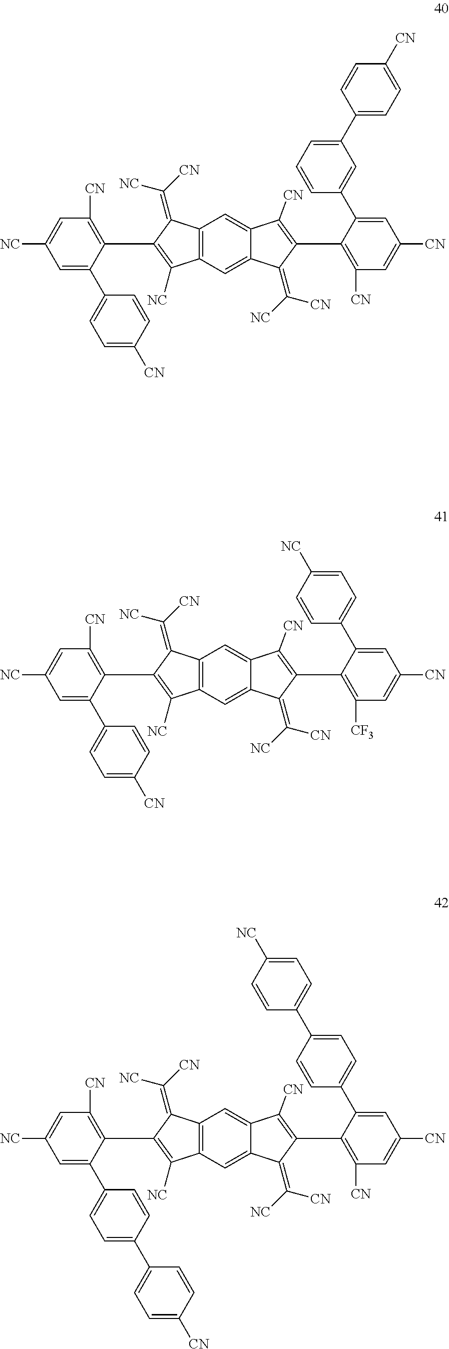 [SMILES: N#CC(C#N)=C1C(c2c(C#N)cc(C#N)cc2-c2ccc(C#N)cc2)=C(C#N)c2cc3c(cc21)C(C#N)=C(c1c(C#N)cc(C#N)cc1-c1cccc(-c2ccc(C#N)cc2)c1)C3=C(C#N)C#N ; N#CC(C#N)=C1C(c2c(C#N)cc(C#N)cc2-c2ccc(C#N)cc2)=C(C#N)c2cc3c(cc21)C(C#N)=C(c1c(-c2ccc(C#N)cc2)cc(C#N)cc1C(F)(F)F)C3=C(C#N)C#N ; N#CC(C#N)=C1C(c2c(C#N)cc(C#N)cc2-c2ccc(-c3ccc(C#N)cc3)cc2)=C(C#N)c2cc3c(cc21)C(C#N)=C(c1c(C#N)cc(C#N)cc1-c1ccc(-c2ccc(C#N)cc2)cc1)C3=C(C#N)C#N]

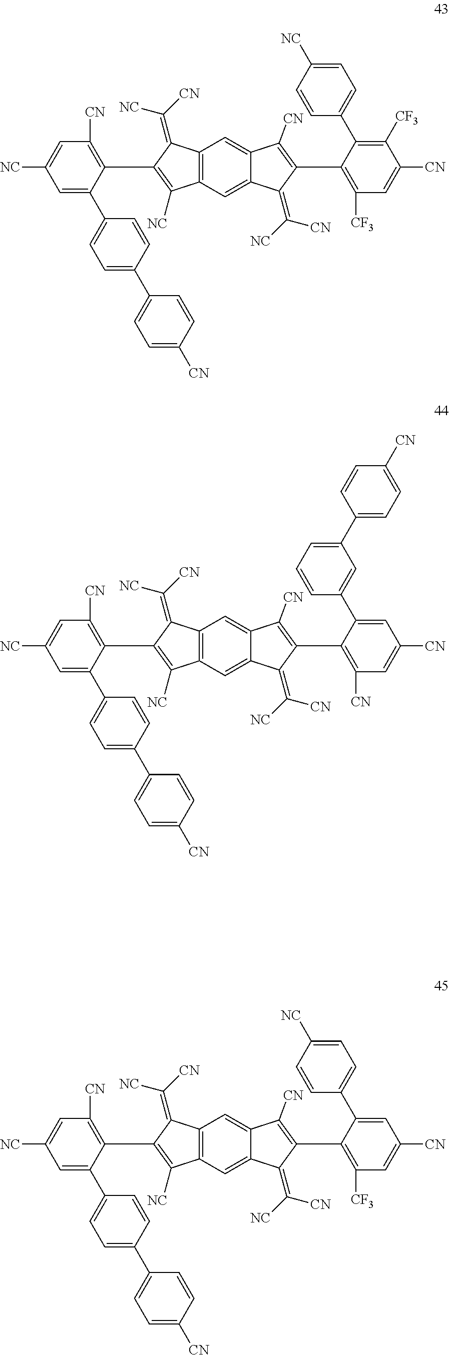 [SMILES: N#CC(C#N)=C1C(c2c(C#N)cc(C#N)cc2-c2ccc(-c3ccc(C#N)cc3)cc2)=C(C#N)c2cc3c(cc21)C(C#N)=C(c1c(C(F)(F)F)cc(C#N)c(C(F)(F)F)c1-c1ccc(C#N)cc1)C3=C(C#N)C#N ; N#CC(C#N)=C1C(c2c(C#N)cc(C#N)cc2-c2ccc(-c3ccc(C#N)cc3)cc2)=C(C#N)c2cc3c(cc21)C(C#N)=C(c1c(C#N)cc(C#N)cc1-c1cccc(-c2ccc(C#N)cc2)c1)C3=C(C#N)C#N ; N#CC(C#N)=C1C(c2c(C#N)cc(C#N)cc2-c2ccc(-c3ccc(C#N)cc3)cc2)=C(C#N)c2cc3c(cc21)C(C#N)=C(c1c(-c2ccc(C#N)cc2)cc(C#N)cc1C(F)(F)F)C3=C(C#N)C#N]

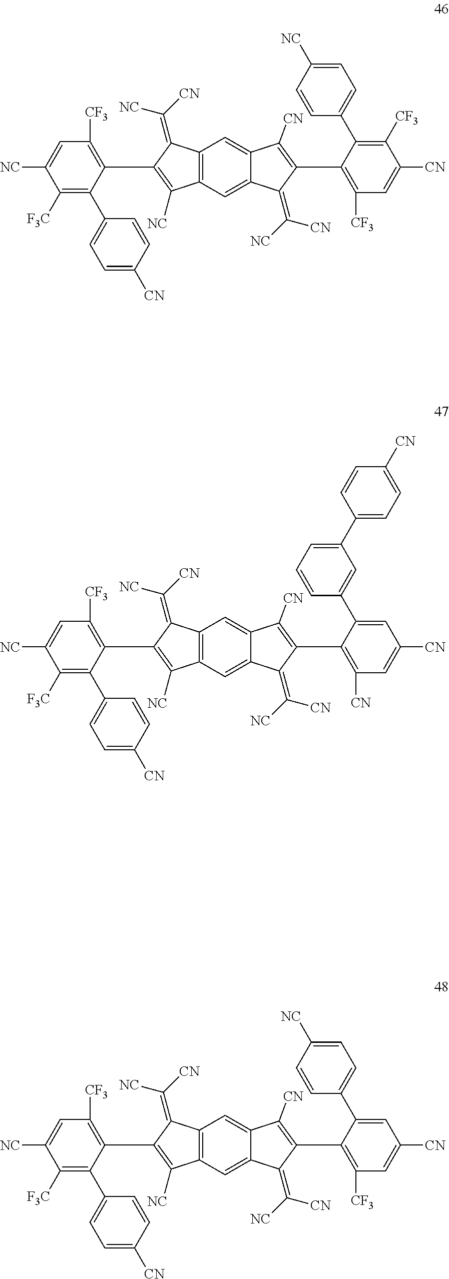 [SMILES: N#CC(C#N)=C1C(c2c(C(F)(F)F)cc(C#N)c(C(F)(F)F)c2-c2ccc(C#N)cc2)=C(C#N)c2cc3c(cc21)C(C#N)=C(c1c(C(F)(F)F)cc(C#N)c(C(F)(F)F)c1-c1ccc(C#N)cc1)C3=C(C#N)C#N ; N#CC(C#N)=C1C(c2c(C#N)cc(C#N)cc2-c2cccc(-c3ccc(C#N)cc3)c2)=C(C#N)c2cc3c(cc21)C(C#N)=C(c1c(C(F)(F)F)cc(C#N)c(C(F)(F)F)c1-c1ccc(C#N)cc1)C3=C(C#N)C#N ; N#CC(C#N)=C1C(c2c(-c3ccc(C#N)cc3)cc(C#N)cc2C(F)(F)F)=C(C#N)c2cc3c(cc21)C(C#N)=C(c1c(C(F)(F)F)cc(C#N)c(C(F)(F)F)c1-c1ccc(C#N)cc1)C3=C(C#N)C#N]

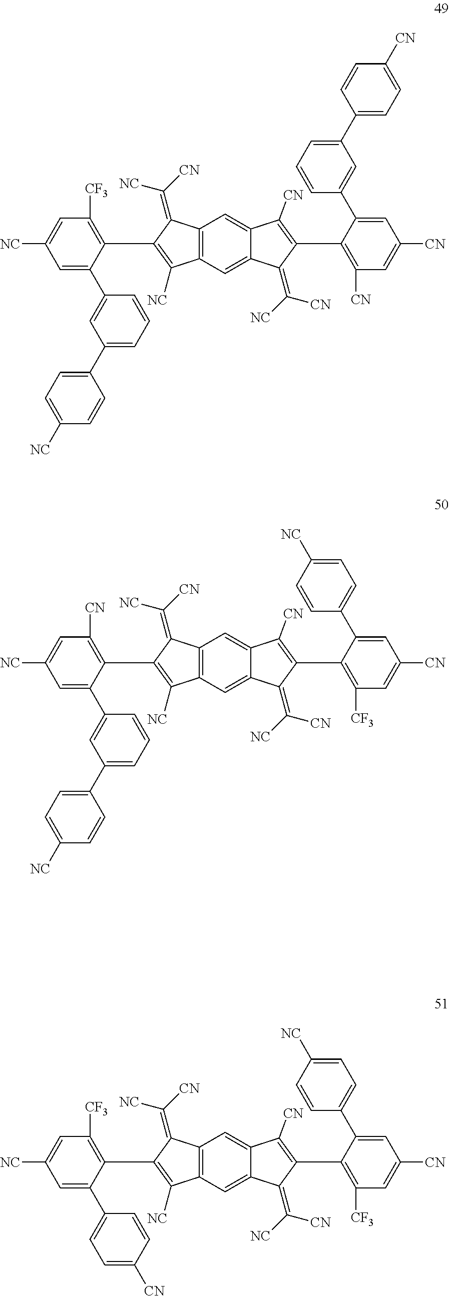 [SMILES: N#CC(C#N)=C1C(c2c(C#N)cc(C#N)cc2-c2cccc(-c3ccc(C#N)cc3)c2)=C(C#N)c2cc3c(cc21)C(C#N)=C(c1c(-c2cccc(-c4ccc(C#N)cc4)c2)cc(C#N)cc1C(F)(F)F)C3=C(C#N)C#N ; N#CC(C#N)=C1C(c2c(C#N)cc(C#N)cc2-c2cccc(-c3ccc(C#N)cc3)c2)=C(C#N)c2cc3c(cc21)C(C#N)=C(c1c(-c2ccc(C#N)cc2)cc(C#N)cc1C(F)(F)F)C3=C(C#N)C#N ; N#CC(C#N)=C1C(c2c(-c3ccc(C#N)cc3)cc(C#N)cc2C(F)(F)F)=C(C#N)c2cc3c(cc21)C(C#N)=C(c1c(-c2ccc(C#N)cc2)cc(C#N)cc1C(F)(F)F)C3=C(C#N)C#N]

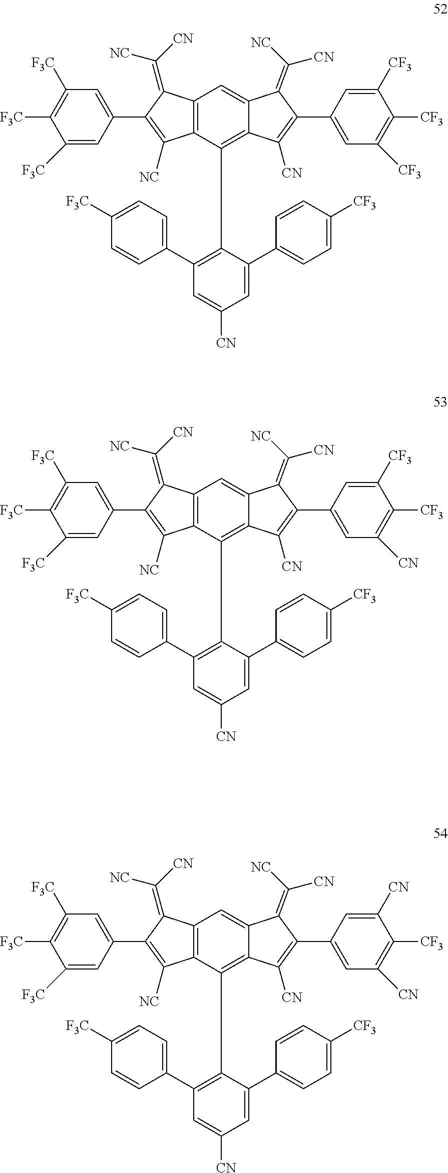

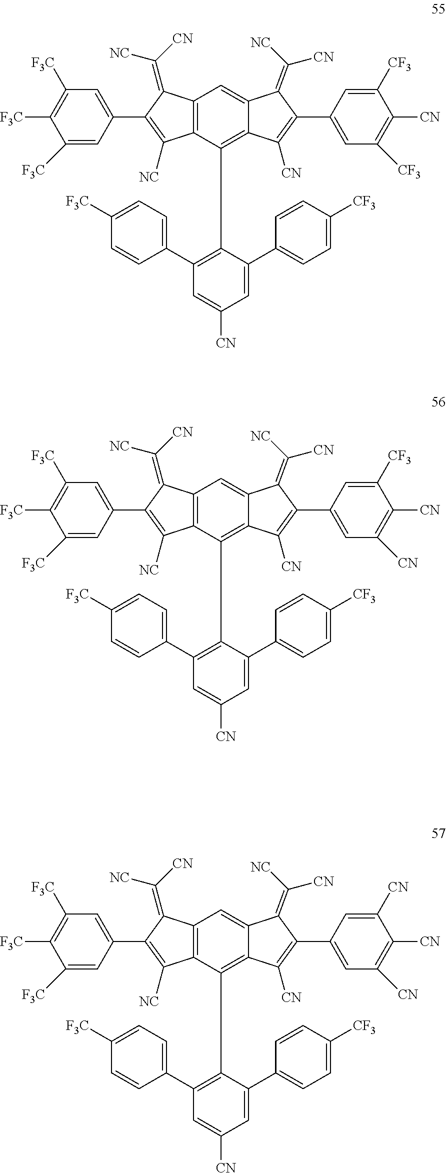

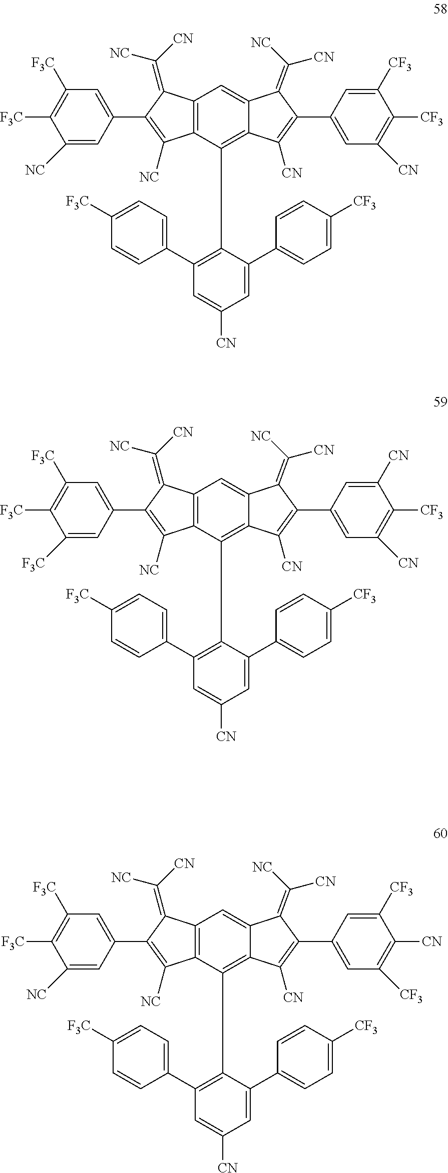

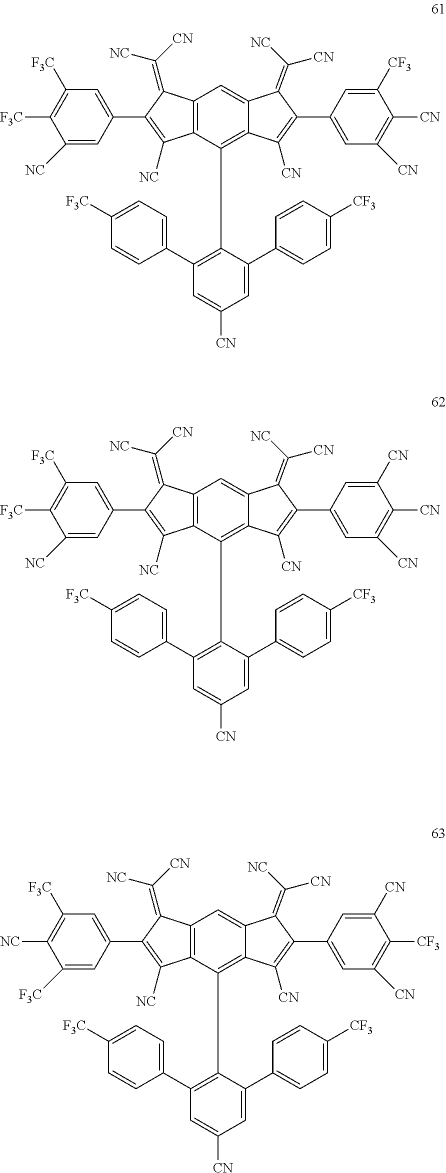

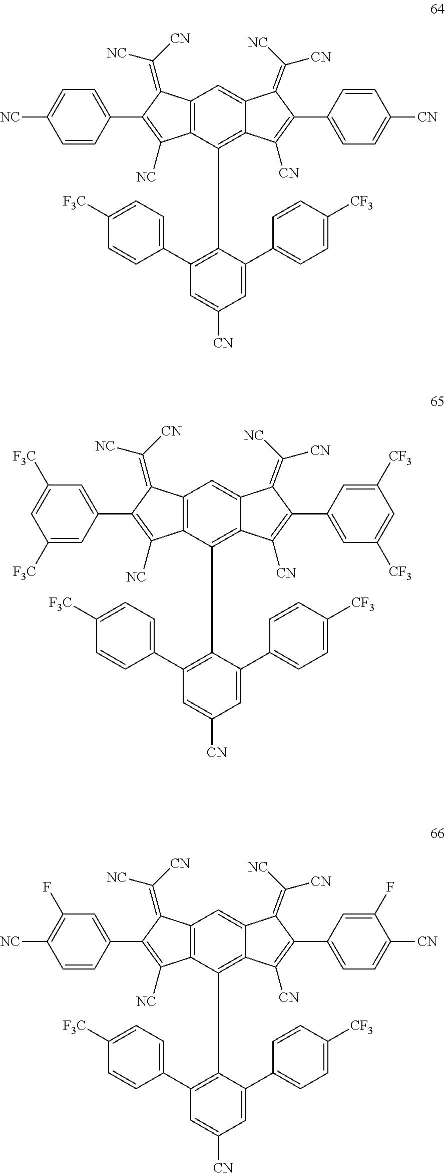 [SMILES: N#CC(C#N)=C1C(c2ccc(C#N)cc2)=C(C#N)c2c1cc1c(c2-c2c(-c3ccc(C(F)(F)F)cc3)cc(C#N)cc2-c2ccc(C(F)(F)F)cc2)C(C#N)=C(c2ccc(C#N)cc2)C1=C(C#N)C#N ; N#CC(C#N)=C1C(c2cc(C(F)(F)F)cc(C(F)(F)F)c2)=C(C#N)c2c1cc1c(c2-c2c(-c3ccc(C(F)(F)F)cc3)cc(C#N)cc2-c2ccc(C(F)(F)F)cc2)C(C#N)=C(c2cc(C(F)(F)F)cc(C(F)(F)F)c2)C1=C(C#N)C#N ; N#CC(C#N)=C1C(c2ccc(C#N)c(F)c2)=C(C#N)c2c1cc1c(c2-c2c(-c3ccc(C(F)(F)F)cc3)cc(C#N)cc2-c2ccc(C(F)(F)F)cc2)C(C#N)=C(c2ccc(C#N)c(F)c2)C1=C(C#N)C#N]

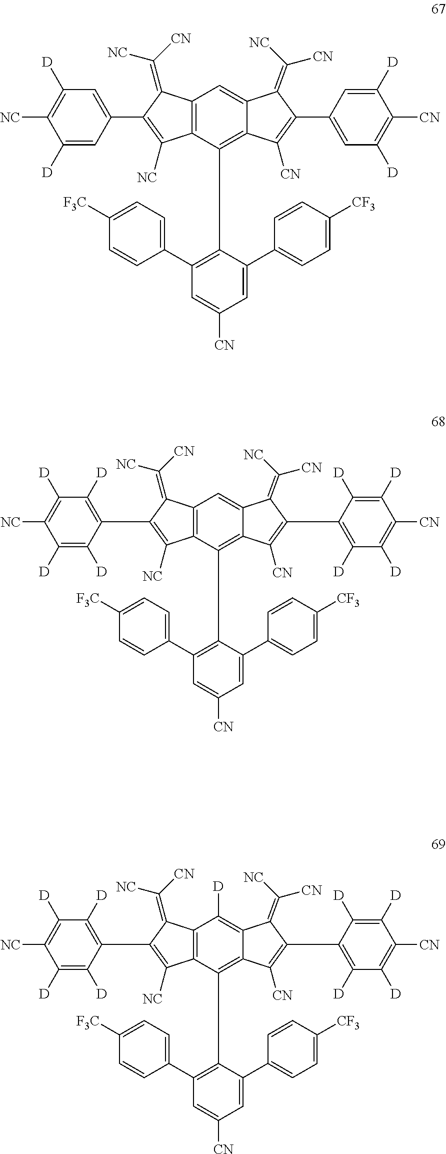

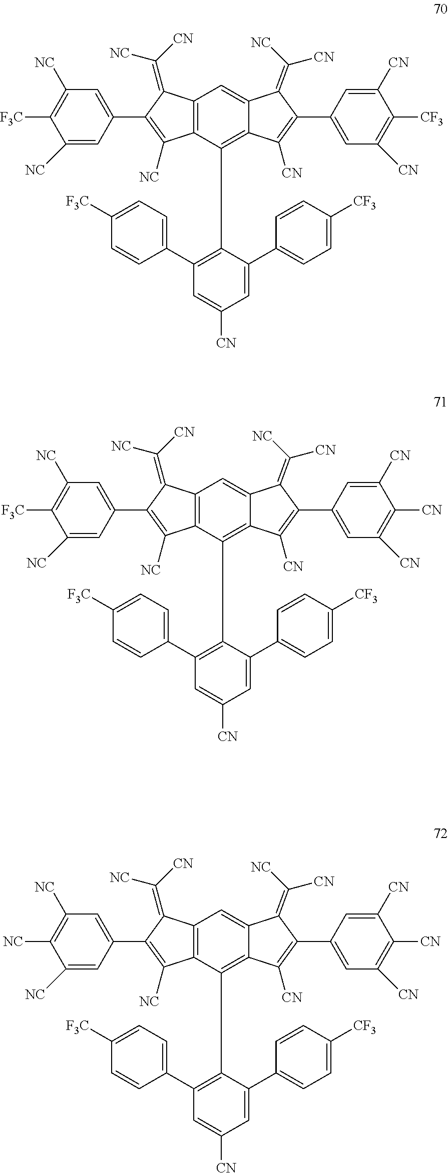 [SMILES: N#CC(C#N)=C1C(c2cc(C#N)c(C(F)(F)F)c(C#N)c2)=C(C#N)c2c1cc1c(c2-c2c(-c3ccc(C(F)(F)F)cc3)cc(C#N)cc2-c2ccc(C(F)(F)F)cc2)C(C#N)=C(c2cc(C#N)c(C(F)(F)F)c(C#N)c2)C1=C(C#N)C#N ; N#CC(C#N)=C1C(c2cc(C#N)c(C#N)c(C#N)c2)=C(C#N)c2c1cc1c(c2-c2c(-c3ccc(C(F)(F)F)cc3)cc(C#N)cc2-c2ccc(C(F)(F)F)cc2)C(C#N)=C(c2cc(C#N)c(C(F)(F)F)c(C#N)c2)C1=C(C#N)C#N ; N#CC(C#N)=C1C(c2cc(C#N)c(C#N)c(C#N)c2)=C(C#N)c2c1cc1c(c2-c2c(-c3ccc(C(F)(F)F)cc3)cc(C#N)cc2-c2ccc(C(F)(F)F)cc2)C(C#N)=C(c2cc(C#N)c(C#N)c(C#N)c2)C1=C(C#N)C#N]

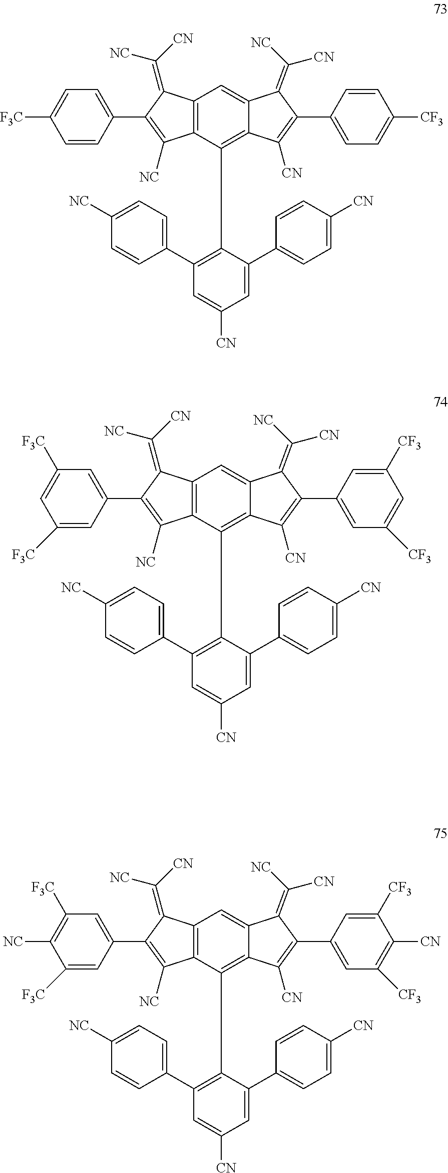 [SMILES: N#CC(C#N)=C1C(c2ccc(C(F)(F)F)cc2)=C(C#N)c2c1cc1c(c2-c2c(-c3ccc(C#N)cc3)cc(C#N)cc2-c2ccc(C#N)cc2)C(C#N)=C(c2ccc(C(F)(F)F)cc2)C1=C(C#N)C#N ; N#CC(C#N)=C1C(c2cc(C(F)(F)F)cc(C(F)(F)F)c2)=C(C#N)c2c1cc1c(c2-c2c(-c3ccc(C#N)cc3)cc(C#N)cc2-c2ccc(C#N)cc2)C(C#N)=C(c2cc(C(F)(F)F)cc(C(F)(F)F)c2)C1=C(C#N)C#N ; N#CC(C#N)=C1C(c2cc(C(F)(F)F)c(C#N)c(C(F)(F)F)c2)=C(C#N)c2c1cc1c(c2-c2c(-c3ccc(C#N)cc3)cc(C#N)cc2-c2ccc(C#N)cc2)C(C#N)=C(c2cc(C(F)(F)F)c(C#N)c(C(F)(F)F)c2)C1=C(C#N)C#N]

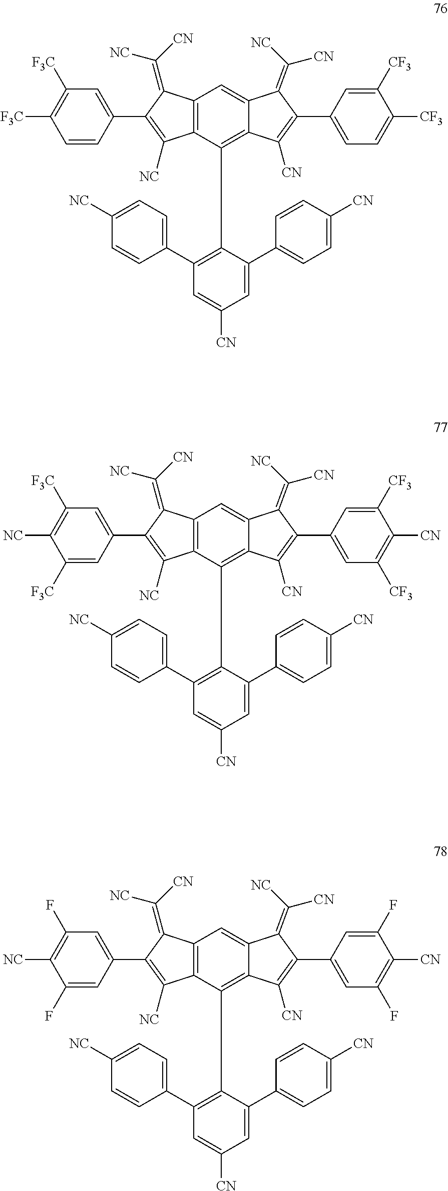 [SMILES: N#CC(C#N)=C1C(c2ccc(C(F)(F)F)c(C(F)(F)F)c2)=C(C#N)c2c1cc1c(c2-c2c(-c3ccc(C#N)cc3)cc(C#N)cc2-c2ccc(C#N)cc2)C(C#N)=C(c2ccc(C(F)(F)F)c(C(F)(F)F)c2)C1=C(C#N)C#N ; N#CC(C#N)=C1C(c2cc(C(F)(F)F)c(C#N)c(C(F)(F)F)c2)=C(C#N)c2c1cc1c(c2-c2c(-c3ccc(C#N)cc3)cc(C#N)cc2-c2ccc(C#N)cc2)C(C#N)=C(c2cc(C(F)(F)F)c(C#N)c(C(F)(F)F)c2)C1=C(C#N)C#N ; N#CC(C#N)=C1C(c2cc(F)c(C#N)c(F)c2)=C(C#N)c2c1cc1c(c2-c2c(-c3ccc(C#N)cc3)cc(C#N)cc2-c2ccc(C#N)cc2)C(C#N)=C(c2cc(F)c(C#N)c(F)c2)C1=C(C#N)C#N]

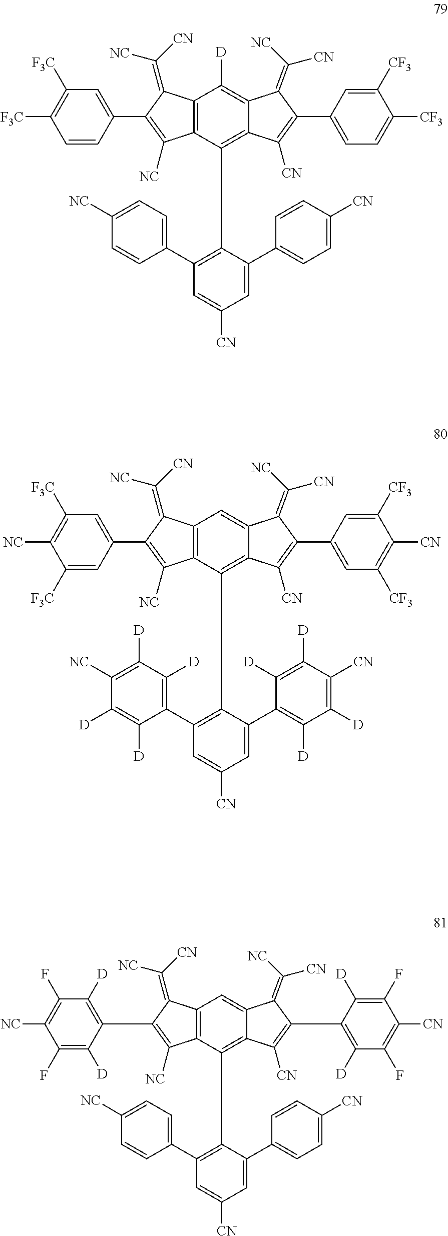

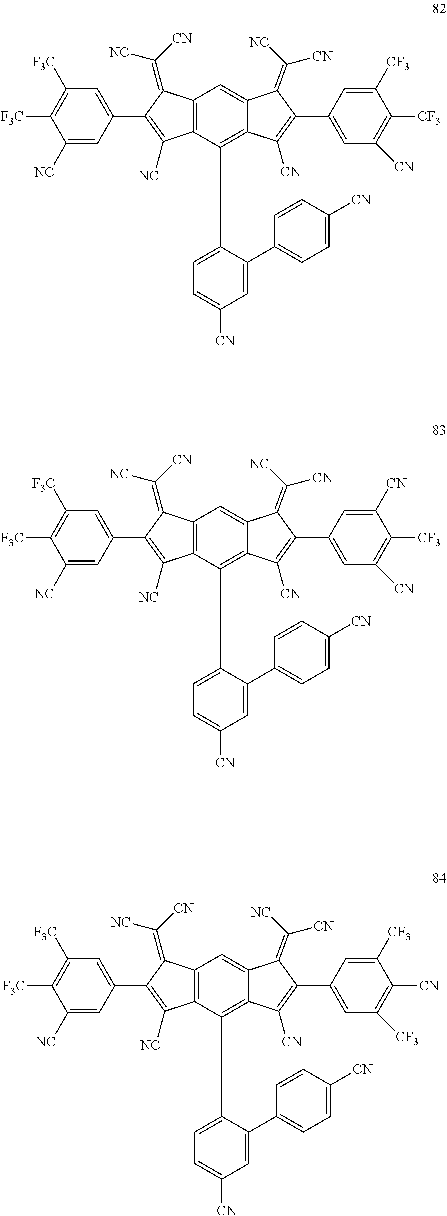 [SMILES: N#CC(C#N)=C1C(c2cc(C#N)c(C(F)(F)F)c(C(F)(F)F)c2)=C(C#N)c2c1cc1c(c2-c2ccc(C#N)cc2-c2ccc(C#N)cc2)C(C#N)=C(c2cc(C#N)c(C(F)(F)F)c(C(F)(F)F)c2)C1=C(C#N)C#N ; N#CC(C#N)=C1C(c2cc(C#N)c(C(F)(F)F)c(C#N)c2)=C(C#N)c2c1cc1c(c2-c2ccc(C#N)cc2-c2ccc(C#N)cc2)C(C#N)=C(c2cc(C#N)c(C(F)(F)F)c(C(F)(F)F)c2)C1=C(C#N)C#N ; N#CC(C#N)=C1C(c2cc(C(F)(F)F)c(C#N)c(C(F)(F)F)c2)=C(C#N)c2c1cc1c(c2-c2ccc(C#N)cc2-c2ccc(C#N)cc2)C(C#N)=C(c2cc(C#N)c(C(F)(F)F)c(C(F)(F)F)c2)C1=C(C#N)C#N]

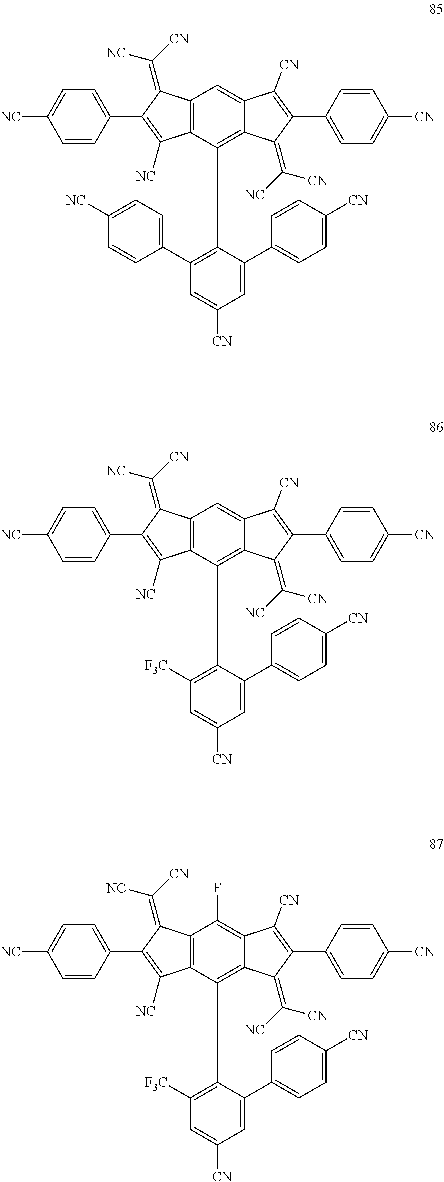 [SMILES: N#CC(C#N)=C1C(c2ccc(C#N)cc2)=C(C#N)c2c1cc1c(c2-c2c(-c3ccc(C#N)cc3)cc(C#N)cc2-c2ccc(C#N)cc2)C(=C(C#N)C#N)C(c2ccc(C#N)cc2)=C1C#N ; N#CC(C#N)=C1C(c2ccc(C#N)cc2)=C(C#N)c2c1cc1c(c2-c2c(-c3ccc(C#N)cc3)cc(C#N)cc2C(F)(F)F)C(=C(C#N)C#N)C(c2ccc(C#N)cc2)=C1C#N ; N#CC(C#N)=C1C(c2ccc(C#N)cc2)=C(C#N)c2c1c(F)c1c(c2-c2c(-c3ccc(C#N)cc3)cc(C#N)cc2C(F)(F)F)C(=C(C#N)C#N)C(c2ccc(C#N)cc2)=C1C#N]

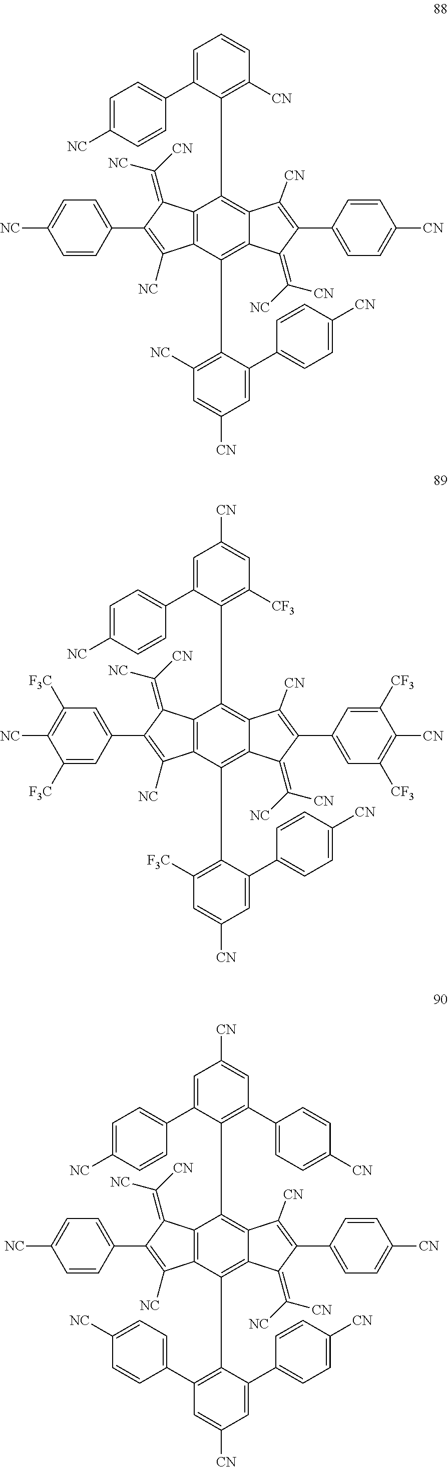

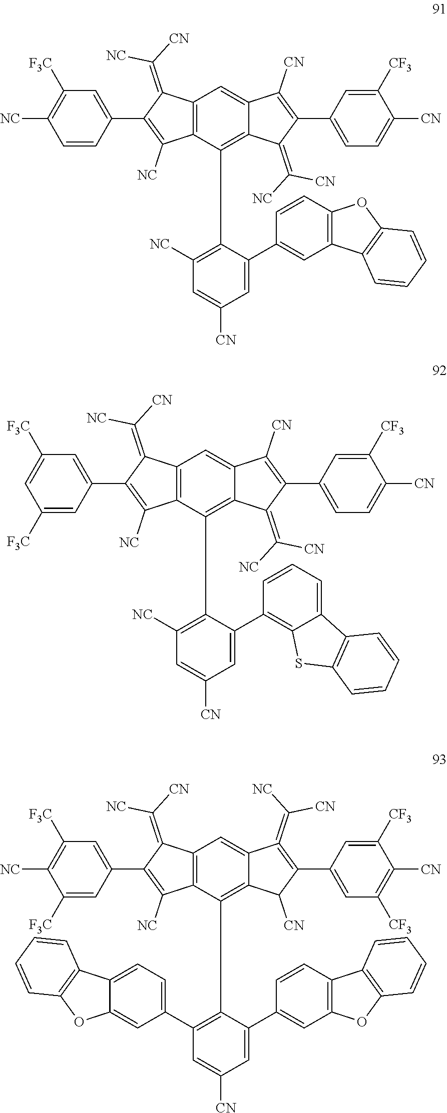

### Light-Emitting Device

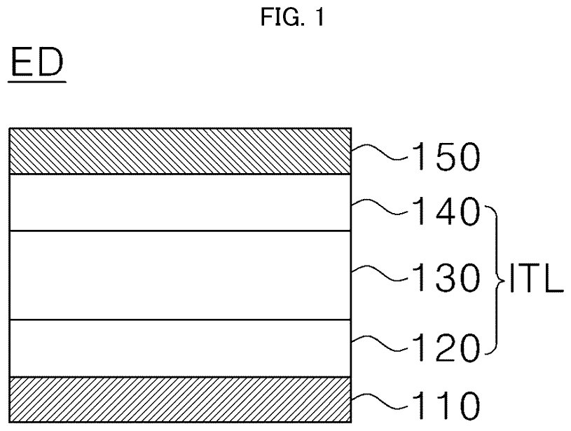

FIGS. 1 to 6 are schematic cross-sectional views illustrating light-emitting devices in accordance with example embodiments.

Referring to FIG. 1, a light-emitting device ED may include a first electrode 110, a second electrode 150, and an emission layer 130 interposed between the first electrode 110 and the second electrode 150. The intermediate layer ITL may include a light-emitting structure including a hole transfer region 120, an emission layer 130, and an electron transfer region 140.

In some embodiments, the intermediate layer ITL may include two or more of the light-emitting structures.

The hole transfer regions 120 may include the above-described polycyclic compound, thereby providing improved driving voltage and life-span properties and sufficient luminous efficiency.

In some embodiments, the hole transfer region 120 may include at least one of a hole injection layer 122, a hole transport layer 124, and an electron blocking layer 126, and the hole injection layer 122 may include the polycyclic compound. Accordingly, hole injection properties and stability of the light-emitting device may be improved.

In some embodiments, the polycyclic compound may include at least one compounds represented by Chemical Formulae 1-1 to 1-10.

In some embodiments, the polycyclic compound may include at least one of the above-mentioned compounds 1 to 93.

In some embodiments, the emission layer 130 may emit a blue light having a maximum emission central wavelength of 430 nm to 490 nm.

A charge generation layer may be included between two or more light-emitting structures, and the charge generation layer may include, e.g., a p-type charge generation layer and/or an n-type charge generation layer.

In some embodiments, the polycyclic compound may be included in an amount of, e.g., about 1 wt % to about 40 wt %, from about 1 wt % to about 30 wt %, based on a total weight of the hole injection layer.

The first electrode 110 may be an anode or a cathode. In some embodiments, the first electrode 110 may serve as an anode, and may serve as a pixel electrode. In this case, the first electrode 110 may include a conductive material with a high work function that promotes hole injection.

In some embodiments, the first electrode 110 may be a transmissive electrode. The first electrode 110 may include a transparent conductive oxide such as indium tin oxide (ITO), indium zinc oxide (IZO), zinc oxide (ZnO), indium tin zinc oxide (ITZO), etc.

In some embodiments, the first electrode 110 may be a translucent electrode or a reflective electrode. The first electrode 110 may include at least one of Ag, Mg, Cu, Al, Pt, Pd, Au, Ni, Nd, Ir, Cr, Li, Ca, Mo, Ti, W, In, Sn, Zn, and an alloy or a compound (e.g., LiF) containing at least one therefrom. For example, the first electrode 110 may include Li, Ca, LiF/Ca (a stacked structure of LiF and Ca), LiF/Al (a stacked structure of LiF and Al), a mixture of Ag and Mg, etc.

The first electrode 110 may have a single-layered structure or a multi-layered structure. For example, the first electrode 110 may have a triple-layered structure of ITO/Ag/ITO.

A thickness of the first electrode 110 may be in a range of about 700 Å to about 10,000 Å. For example, the thickness of the first electrode 110 may be in a range of about 1,000 Å to about 3,000 Å.

The second electrode 150 may be a cathode or an anode. In some embodiments, the second electrode 150 may serve as an electron injection electrode or as a cathode. The second electrode 150 may include a metal, an alloy, an electrically conductive compound, etc., having a low work function.

For example, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, etc. The second electrode 150 may include one of the aforementioned materials, or a combination thereof.

The second electrode 150 may be a transmissive electrode, a translucent electrode, or a reflective electrode. The second electrode 150 may have a single-layered structure or a multi-layered structure.

The emission layer 130 may include a host material and a dopant.

In a non-limiting example, the emission layer 130 may include the dopant in an amount of about 0.01 parts by weight to about 15.00 parts by weight, or about 0.01 parts by weight to about 12.00 parts by weight, based on 100 parts by weight of the host material.

The emission layer 130 may emit a red light, a green light, a blue light and/or a white light. For example, the emission layer 130 may emit a blue light.

In some embodiments, an emission half width (full width at half maximum) of the blue light may be about 30 nm or less, or about 28 nm or less.

For example, the emission layer 130 may include the host material such as an anthracene derivative, a pyrene derivative, a fluoranthene derivative, a chrysene derivative, a dihydrobenzanthracene derivative, a triphenylene derivative, or the like.

In some embodiments, the emission layer 130 may include, e.g., a host material represented by Chemical Formula FH. For example, the compound represented by Chemical Formula FH may be used as a fluorescent host material.

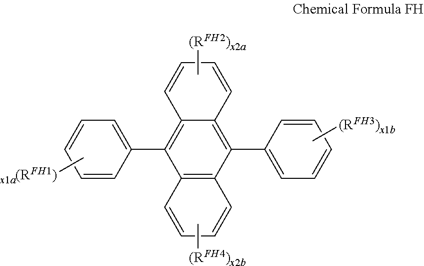

In Chemical Formula FH, R^{FH1} to R^{FH4} may each independently be a hydrogen atom, a deuterium atom, a halogen atom, a substituted or unsubstituted silyl group, a substituted or unsubstituted thio group, a substituted or unsubstituted oxy group, a substituted or unsubstituted C₁-C₁₀ alkyl group, a substituted or unsubstituted C₂-C₁₀ alkenyl group, a substituted or unsubstituted C₆-C₃₀ aryl group, a substituted or unsubstituted C₆-C₆₀ alkyl aryl group, a substituted or unsubstituted C₆-C₆₀ aryl alkyl group, a substituted or unsubstituted C₆-C₃₀ heteroaryl group, a substituted or unsubstituted C₃-C₆₀ alkyl heteroaryl group, a substituted or unsubstituted C₃-C₆₀ heteroarylalkyl group, or a cyclic group formed through a combination thereof. In some embodiments, in Chemical Formula FH, at least one of R^{FH1} to R^{FH4} may form a condensed ring with a bonded benzene ring.

In Chemical Formula FH, x1a and x1b may each independently be an integer from 0 to 5; and x2a and x2b may each independently be an integer from 0 to 4. When x1a, x1b, x2a, and x2b are each 2 or more, two or more of each of R^{FH1} to R^{FH4} may be the same as or different from each other.

In some embodiments, the emission layer 130 may include, e.g., a host material represented by Chemical Formula PH. For example, the compound represented by Chemical Formula PH may be used as a host material for a phosphorescent device.

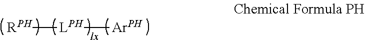

In Chemical Formula PH, R^{PH} may be a substituted or unsubstituted carbazole group. L^{PH} may be a direct linkage, a substituted or unsubstituted C₆-C₃₀ arylene group, or a substituted or unsubstituted C₂-C₃₀ heteroarylene group. Ar^{PH} may be a substituted or unsubstituted C₆-C₃₀ aryl group, a substituted or unsubstituted C₆-C₆₀ alkyl aryl group, a substituted or unsubstituted C₆-C₆₀ aryl alkyl group, a substituted or unsubstituted C₂-C₃₀ heteroaryl group, a substituted or unsubstituted C₃-C₆₀ alkyl heteroaryl group, or a substituted or unsubstituted C₃-C₆₀ heteroarylalkyl group.

As described above in the definition of terminology, the term “C₆-C₃₀ aryl group” may encompass a group in which multiple aryl rings are condensed or bonded through a cyclic group (e.g., an alicyclic hydrocarbon ring). For example, a C₆-C₃₀ aryl group may include a fluorenyl group.

As described above in the definition of terminology, the term “C₂-C₃₀ heteroaryl group” may encompass a group in which multiple aryl rings are condensed or bonded through a heterocyclic ring. For example, a C₂-C₃₀ heteroaryl group may include a carbazole group, a dibenzofuran group, a dibenzothiophene group, etc. In some embodiments, a C₂-C₃₀ heteroaryl group may be a group in which multiple aryl rings are condensed or bonded to each other through the same or different heterocyclic rings.

In some embodiments, a substituent included in Ar^{PH} may be a silyl group represented by —Si(R^{sa})(R^{sb})(R^{sc}); and R^{sa}, R^{sb}, and R^{sc} may each independently be hydrogen, halogen, a hydroxyl group, a C₁-C₆₀ alkyl group, a C₁-C₆₀ alkoxy group, a C₁-C₆₀ alkylthio group, a C₆-C₆₀ aryl group, or a C₂-C₃₀ heteroaryl group. At least one of R^{sa}, R^{sb}, and R^{sc} may be a C₆-C₆₀ aryl group or a C₂-C₃₀ heteroaryl group. For example, R^{sa}, R^{sb} and R^{sc} may each independently be a C₆-C₆₀ aryl group or a C₂-C₃₀ heteroaryl group.

In Chemical Formula PH, lx may be an integer from 0 to 10. When lx is 2 or more, two or more of L^{PH} may be the same as or different from each other.

The emission layer 130 may include, e.g., BCPDS (bis (4-(9H-carbazol-9-yl) phenyl) diphenylsilane), POPCPA ((4-(1-(4-(diphenylamino) phenyl) cyclohexyl) phenyl) diphenyl-phosphine oxide), DPEPO (bis[2-(diphenylphosphino)phenyl]ether oxide), mCBP (3,3′-di(9H-carbazol-9-yl)-1,1′-biphenyl), CBP (4,4′-bis(N-carbazolyl)-1,1′-biphenyl), mCP (1,3-bis(carbazol-9-yl)benzene), PPF (2,8-bis(diphenylphosphoryl) dibenzo[b,d]furan), TCTA (4,4′,4″-tris(carbazol-9-yl)-triphenylamine), TPBi (1,3,5-tris(1-phenyl-1H-benzo[d]imidazole-2-yl)benzene), Alq3 (tris(8-hydroxyquinolino) aluminum), ADN (9,10-di(naphthalene-2-yl)anthracene), TBADN (2-tert-butyl-9,10-di(naphth-2-yl)anthracene), DSA (distyrylarylene), CDBP (4,4′-bis(9-carbazolyl)-2,2′-dimethyl-biphenyl), MADN (2-methyl-9,10-bis(naphthalen-2-yl)anthracene), CP1 (hexaphenyl cyclotriphosphazene), UGH2 (1,4-bis(triphenylsilyl)benzene), DPSiO3 (hexaphenylcyclotrisiloxane), DPSiO4 (octaphenylcyclotetrasiloxane), or the like, as the host material.

In some embodiments, in the emission layer 130, the host material may include one of the materials as described above, or any combination thereof.

Non-limiting examples of the host material are as follows.

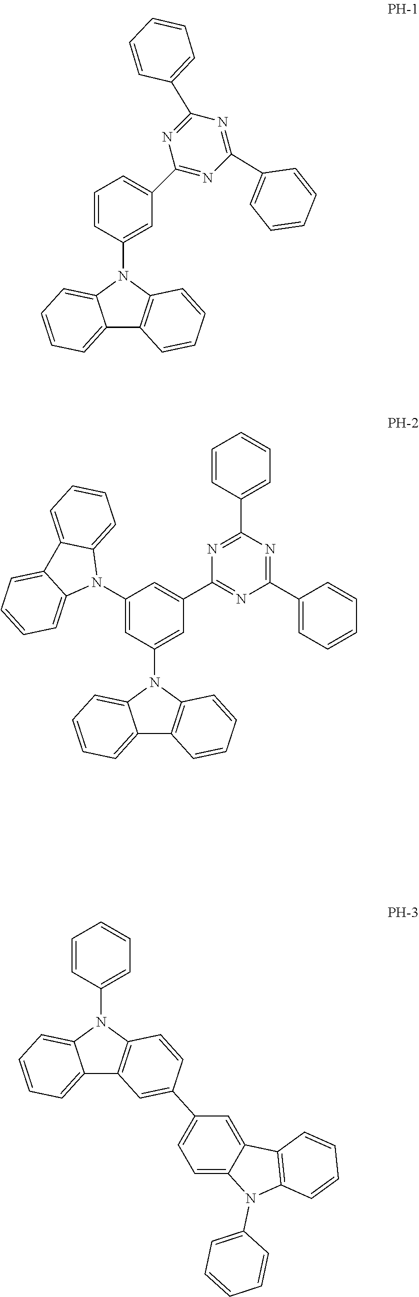 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1ccc(-n2c3ccccc3c3cc(-c4ccc5c(c4)c4ccccc4n5-c4ccccc4)ccc32)cc1]

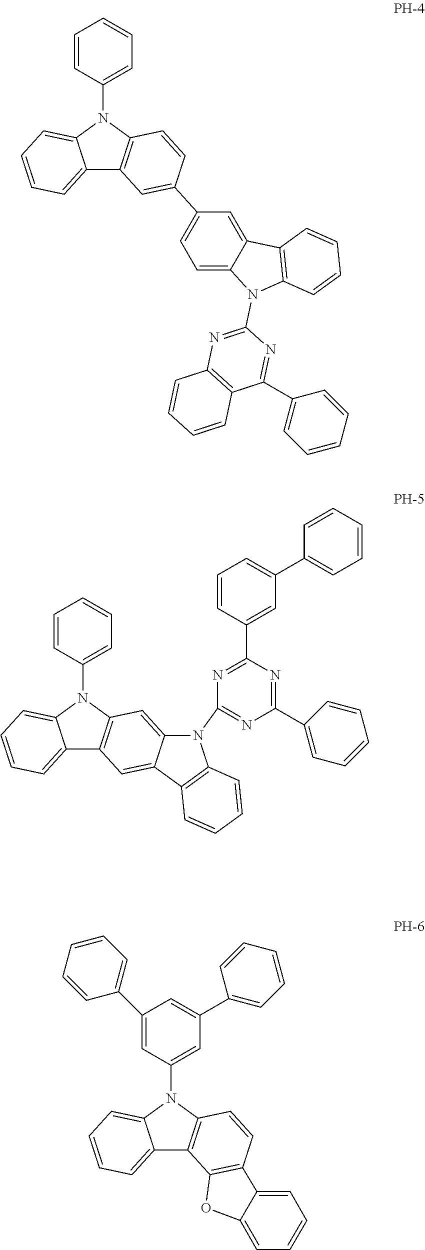 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-c3ccccc3)cc(-n3c4ccccc4c4c5oc6ccccc6c5ccc43)c2)cc1]

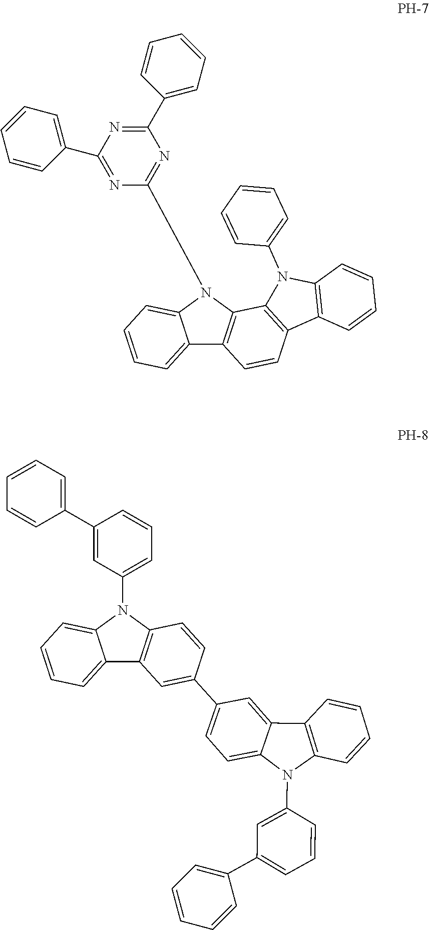 [SMILES: c1ccc(-c2nc(-c3ccccc3)nc(-n3c4ccccc4c4ccc5c6ccccc6n(-c6ccccc6)c5c43)n2)cc1 ; c1ccc(-c2cccc(-n3c4ccccc4c4cc(-c5ccc6c(c5)c5ccccc5n6-c5cccc(-c6ccccc6)c5)ccc43)c2)cc1]

The emission layer 130 may further include the dopant interacting with the host material.

In some embodiments, the emission layer 130 may include a dopant represented by Chemical Formula FD. For example, the compound represented by Chemical Formula FD may be used as a fluorescent dopant.

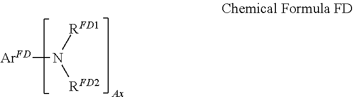

In Chemical Formula FD, Ar^{FD}, R^{FD1}, and R^{FD2} may each independently be a substituted or unsubstituted C₃-C₆₀ carbocyclic group, or a substituted or unsubstituted C₁-C₆₀ heterocyclic group. Ax may be an integer from 1 to 6.

In some embodiments, Ar^{FD} may include a condensed ring structure in which three or more aryl rings or benzene rings are condensed together (e.g., an anthracene group, a chrysene group, a pyrene group, etc.).

In some embodiments, the emission layer 130 may include a phosphorescent dopant. The phosphorescent dopant may include an organometallic compound that includes a central metal and at least one ligand bonded to the central metal via a coordination bond. The central metal may include, e.g., a transition metal, and the ligand may include, e.g., a monodentate ligand, a bidentate ligand, a tridentate ligand, a tetradentate ligand, a pentadentate ligand, a hexadentate ligand, or a combination thereof.

The phosphorescent dopant may include, e.g., a compound represented by Chemical Formula PD.

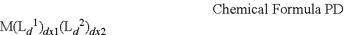

In Chemical Formula PD, M may be a transition metal atom, e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), ruthenium (Ru), copper (Cu), or thulium (Tm).

In Chemical Formula PD, L_{d}¹ may be a ligand represented by Chemical Formula LD1.

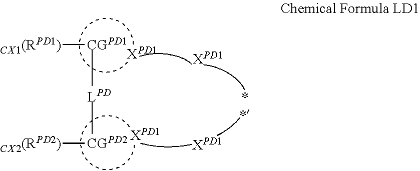

In Chemical Formula LD1, X^{PD1} and X^{PD2} may each independently be C or N.

In some embodiments, one of X^{PD1} and X^{PD2} may be C and the other may be N. In some embodiments, X^{PD1} and X^{PD2} may each be N.

In Chemical Formula LD1, CG^{PD1} and CG^{PD2} may each independently be a substituted or unsubstituted C₃-C₆₀ carbocyclic group, or a substituted or unsubstituted C₁-C₆₀ heterocyclic group. For example, CG^{PD1} and CG^{PD2} may each independently be a pyrrole group, a pyrazole group, an imidazole group, a triazole group, an oxazole group, an isoxazole group, a thiazole group, an isothiazole group, an oxadiazole group or a thiadiazole group, a benzene group, a pyridine group, a pyrimidine group, a naphthalene group, a dibenzofuran group, a dibenzothiophene group, a carbazole group, a fluorene group, a dibenzosilole group, a naphthobenzofuran group, a naphthobenzothiophene group, a benzocarbazole group, a benzofluorene group, a naphthobenzosilole group, a dinaphthofuran group, a dinaphthothiophene group, a dibenzocarbazole group, a dibenzofluorene group, a dinaphthosilole group, an azadibenzofuran group, an azadibenzothiophene group, an azacarbazole group, an azafluorene group, an azadibenzosilole group, an azanaphthobenzofuran group, an azanaphthobenzothiophene group, an azabenzocarbazole group, an azabenzofluorene group, an azanaphthobenzosilole group, an azadinapthofuran group, an azadinapthothiophene group, an azadibenzocarbazole group, an azadibenzofluorene group, or an azadinapthosilole group.

In Chemical Formula LD1, L^{PD} may be a single bond, a substituted or unsubstituted methylene group, a substituted or unsubstituted ethylene group, *—O—*′, *—S—*′, *—C(═O)—*′, *—N(R^{PD3})—*′, *—C(R^{PD4})═*′, or *═C(R^{PD5})—*′.

In Chemical Formula LD1, X^{PD3} and X^{PD4} may each independently be a chemical bond, O, S, N(R^{PD6}), B(R^{PD7}), P(R^{PD8}), C(R^{PD9})(R^{PD10}), or Si(R^{PD11})(R^{PD12}). The chemical bond may be, e.g., a covalent bond or a coordination bond.

In Chemical Formula LD1, R^{PD1} and R^{PD2} may each independently be hydrogen, deuterium, —F, —Cl, —Br, —I, —SF₅, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, a substituted or unsubstituted C₈-C₆₀ condensed polycyclic group, a substituted or unsubstituted silyl group, a substituted or unsubstituted amine group, a substituted or unsubstituted aniline group, —B(R^{PD13})(R^{PD14}), —C(═O)(R^{PD15}), —S(═O)₂(R^{PD16}), or —P(═O)(R^{PD17})(R^{PD18}). The silyl group may be represented by —Si(R^{sa})(R^{sb})(R^{sc}), as explained above.

R^{PD3} to R^{PD18} may each independently be hydrogen, deuterium, —F, —Cl, —Br, —I, —SF₅, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

In Chemical Formula LD1, cx1 and cx2 may each independently be an integer from 0 to 10. When at least one of cx1 and cx2 is 2 or more, two or more of R^{PD1} or two or more of R^{PD2} may be the same as or different from each other.

The symbols -* and -*′ each represent a binding site where the ligand represented by Chemical Formula LD1 bonds to M.

In Chemical Formula PD, dx1 may be an integer from 1 to 3. When dx1 is 2 or 3, two or three of L_{d}^{a} may be the same as or different from each other. Among two or three of L_{d}¹, CG^{PD1} and/or CG^{PD2} adjacent to each other may be connected to each other through a connecting group such as L^{PD1}, L^{PD2}, etc. The connecting group, such as L^{PD1}, L^{PD2}, etc., may each independently be the same as defined in connection with L^{PD}.

In Chemical Formula PD, L_{d}² may be an organic ligand. L_{d}² may include, e.g., a halogen group, CO, NO, CS, picolinate, acetate, oxalate, a diketone group, an isonitrile group, isothiocyanato-N, thiosulphato-S, an alkyl phosphine, phenylphosphine, an aryl phosphine, phosphine oxide, phosphite, or a combination thereof.

In Chemical Formula PD, dx2 is an integer of 0 to 4. When dx2 is 2 or more, two or more of L_{d2} may be the same as or different from each other.

Non-limiting examples of the phosphorescent dopant are as follows.

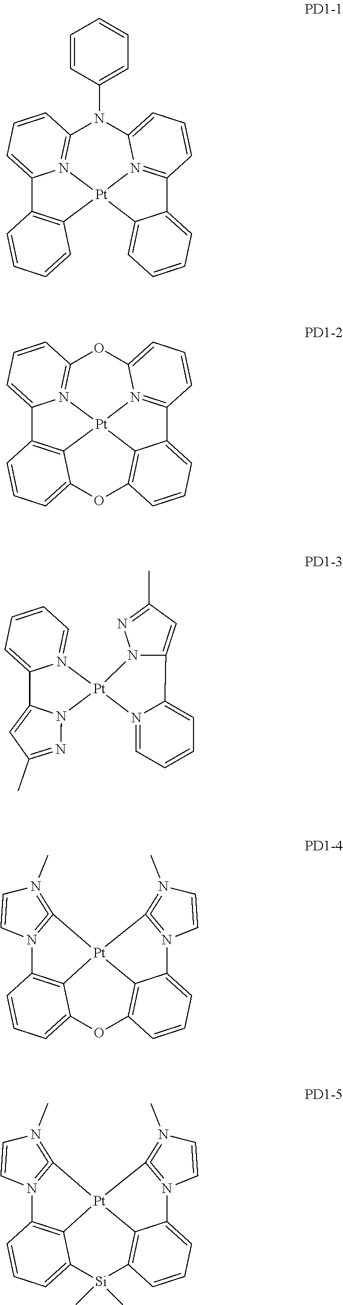

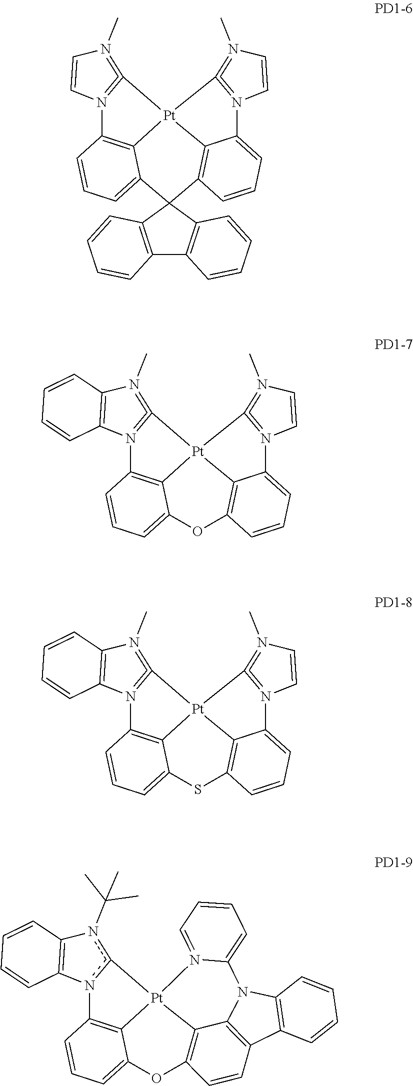

In some embodiments, the emission layer 130 may include a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (NBDAVBi), etc.), 4,4′-bis[2-(4-(N,N-diphenylamino)phenyl)vinyl]biphenyl (DPAVBi), perylene or a derivative thereof (e.g., 2,5,8,11-tetra-t-butylperylene (TBP)), pyrene or a derivative thereof (e.g., 1,1-dipyrene, 1,4-dipyrenylbenzene, 1,4-bis(N,N-diphenylamino)pyrene), etc.), etc., as a fluorescent dopant.

The emission layer 130 may include a metal complex that includes iridium (Ir), platinum (Pt), osmium (Os), gold (Au), titanium (Ti), zirconium (Zr), hafnium (Hf), europium (Eu), terbium (Tb), or thulium (Tm) as a phosphorescent dopant, in addition to the materials described above. For example, FIrpic (iridium(III) bis(4,6-difluorophenylpyridinato-N,C2′)picolinate), FIr6 (bis(2,4-difluorophenylpyridinato)-tetrakis(1-pyrazolyl)borate iridium(III)), PtOEP (platinum octaethyl porphyrin), etc., may be used as the phosphorescent dopant.

In some embodiments, the emission layer 130 may include a boron-containing dopant represented by Chemical Formula BD.

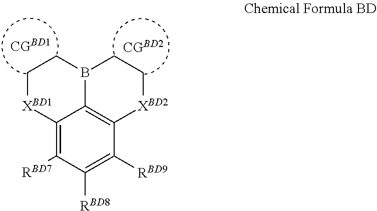

In Chemical Formula BD, X^{BD1} and X^{BD2} may each independently be N(R^{BD1}), P(R^{BD2}) C(R^{BD3}) (R^{BD4}), Si(R^{BD5})(R^{BD6}), S, or O. In some embodiments, X^{BD1} and X^{BD2} may each be N(R^{BD1}). R^{BD1} to R^{BD6} may each independently be hydrogen, deuterium, a substituted or unsubstituted C₁-C₂₀ alkyl group, a substituted or unsubstituted C₆-C₃₀ aryl group, or a substituted or unsubstituted C₂-C₃₀ heteroaryl group. R^{BD7}, R^{BD8}, and R^{BD9} may each independently be hydrogen, deuterium, halogen, a cyano group, a substituted or unsubstituted amine group, a substituted or unsubstituted boryl group, a substituted or unsubstituted oxy group, a substituted or unsubstituted thio group, a substituted or unsubstituted C₁-C₂₀ alkyl group, a substituted or unsubstituted C₆-C₃₀ aryl group, or a substituted or unsubstituted C₂-C₃₀ heteroaryl group. R^{BD7}, R^{BD8} and/or R^{BD9} may be bonded to an adjacent group to form a ring.

In Chemical Formula BD, CG^{BD1} and CG^{BD2} represent a cyclic group, and CG^{BD1} and CG^{BD2} may each independently be a substituted or unsubstituted C₃-C₆₀ carbocyclic group, or a substituted or unsubstituted C₁-C₆₀ heterocyclic group. In some embodiments, CG^{BD1} and CG^{BD2} may each independently be a substituted or unsubstituted C₆-C₃₀ aryl group, or a substituted or unsubstituted C₂-C₃₀ heteroaryl group.

In some embodiments, CG^{BD1} and CG^{BD2} may each independently be a substituted or unsubstituted benzene ring. In this case, the boron-containing dopant may serve as a thermally activated delayed fluorescence (TADF) dopant.

In some embodiments, one of CG^{BD1} and CG^{BD2} may be a non-condensed aryl group or a non-condensed heteroaryl group, and the other one thereof may be a condensed polycyclic aryl group or a condensed polycyclic heteroaryl group. In this case, the boron-containing dopant may serve as a fluorescent dopant.

In some embodiments, the emission layer 130 may include one of the dopant materials as described above, or any combination thereof.

In some embodiments, the emission layer 130 may include two or more host materials. For example, the emission layer 130 may include a hole transporting host and an electron transporting host. In this case, the emission layer 130 may include a hole transporting host, an electron transporting host, a photosensitive agent, and a dopant. In some embodiments, the hole transporting host and the electron transporting host may form an exciplex, and energy may be transferred from the exciplex to the photosensitive agent and from the photosensitive agent to the dopant, thereby inducing a light emission.

Non-limiting examples of the hole transporting host may include a compound represented by Chemical Formula HT as described below. Non-limiting examples of the electron transporting host may include a compound represented by Chemical Formula ET as described below.

In some embodiments, the emission layer 130 may include quantum dots. A quantum dot may include a Group II-VI compound, a Group III-VI compound, a Group I-III-VI compound, a Group III-V group compound, a Group III-II-V group compound, a Group IV-VI compound, a Group IV element, a Group IV compound, or a combination thereof.

The quantum dot may include a core that includes the compound as described above, and a shell surrounding the core. The shell may include an inorganic oxide or a semiconductor compound. Examples of the semiconductor compound may include CdS, CdSe, CdTe, ZnS, ZnSe, ZnTe, ZnSe, ZnTeS, GaAs, GaP, GaSb, HgS, HgSe, HgTe, InAs, InP, InGaP, InSb, AlAs, AlP, AlSb, etc.

In some embodiments, a color of light from a quantum dot may be adjusted according to a particle size of the quantum dot. The quantum dot may include a blue quantum dot, a red quantum dot, or a green quantum dot.

The hole transfer region 120 may be formed between the first electrode 110 and the emission layer 130. The hole transfer region 120 may have a single-layered structure or a multi-layered structure including different materials.

The hole transfer region 120 may include the above-described polycyclic compound.

The hole transfer region 120 may include a hole injection layer, a hole transport layer, and/or an electron blocking layer, and may further include an auxiliary emission layer.

The hole injection layer may include the above-described polycyclic compound.

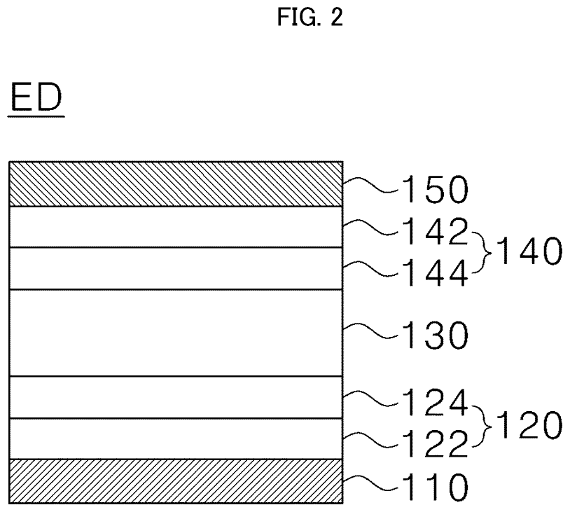

In some embodiments, as illustrated in FIG. 2, the hole transfer region 120 may include a hole injection layer 122 and a hole transport layer 124, sequentially stacked from the first electrode 110.

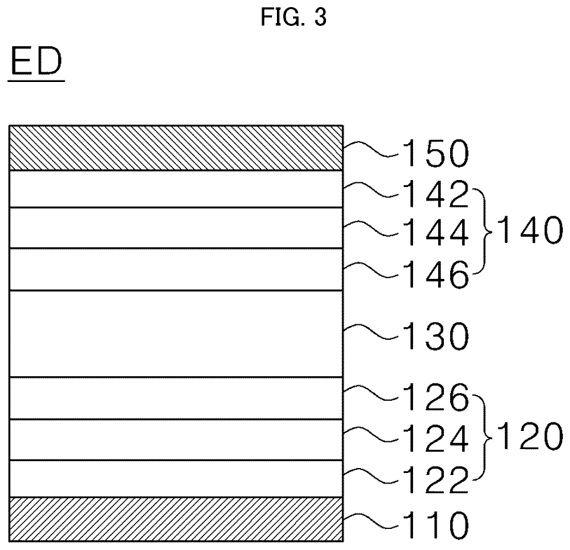

In some embodiments, as illustrated in FIG. 3, the hole transfer region 120 may include a hole injection layer 122, a hole transport layer 124, and an electron blocking layer 126, sequentially stacked from the first electrode 110. The electron blocking layer 126 may block an electron transfer from the electron transfer region 140 to the hole transfer region 120. Accordingly, the generation of excitons in the emission layer 130 may be increased, and light-emission efficiency may be further increased.

In some embodiments, the hole transfer region 120 may include a compound as described below.

For example, the hole transfer region 120 may include a compound represented by Chemical Formula HT.

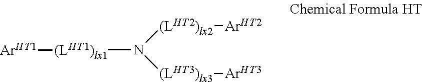

In Chemical Formula HT, L^{HT1}, L^{HT2}, and L^{HT3} may each independently be a direct linkage, a substituted or unsubstituted C₆-C₃₀ arylene group, or a substituted or unsubstituted C₂-C₃₀ heteroarylene group.

In Chemical Formula HT, lx1 to lx3 may each independently be an integer from 0 to 10. When lx1, lx2 or lx3 is 2 or more, two or more of each of L^{HT1} L^{HT2}, or L^{HT3} may be directly connected by, e.g., carbon atoms (e.g., sp2 carbons) of each aryl ring, to form a substituted or unsubstituted C₆-C₃₀ arylene group, or a substituted or unsubstituted C₂-C₃₀ heteroarylene group.

In Chemical Formula HT, Ar^{HT1} and Ar^{HT2} may each independently be a substituted or unsubstituted C₆-C₃₀ aryl group, or a substituted or unsubstituted C₂-C₃₀ heteroaryl group. Ar^{HT3} may be a substituted or unsubstituted C₆-C₃₀ aryl group.

In some embodiments, the compound represented by Chemical Formula HT may be a monoamine compound. In some embodiments, the compound represented by Chemical Formula HT may be a diamine compound in which at least one of Ar^{HT1} to Ar^{HT3} includes an amine group as a substituent.

In some embodiments, the compound represented by Chemical Formula HT may be a carbazole-based compound in which at least one of Ar^{HT1} and Ar^{HT2} includes a substituted or unsubstituted carbazole group. or a fluorene-based compound in which at least one of Ar^{HT1} and Ar^{HT2} includes a substituted or unsubstituted fluorene group.

In some embodiments, two adjacent groups among Ar^{HT1} to Ar^{HT3} may be condensed together to form a ring.

In non-limiting examples, the hole transfer region 120 may include at least one compound as follows.

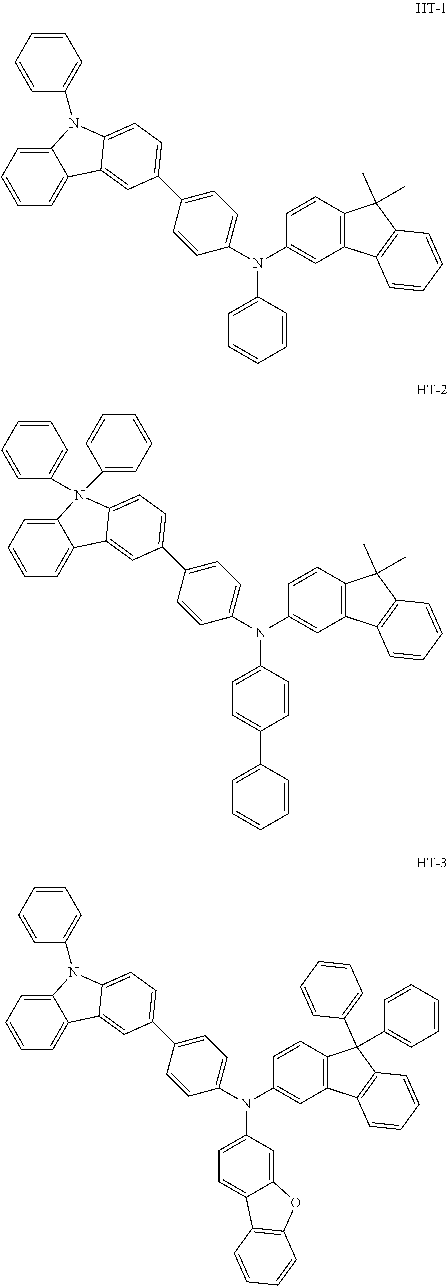

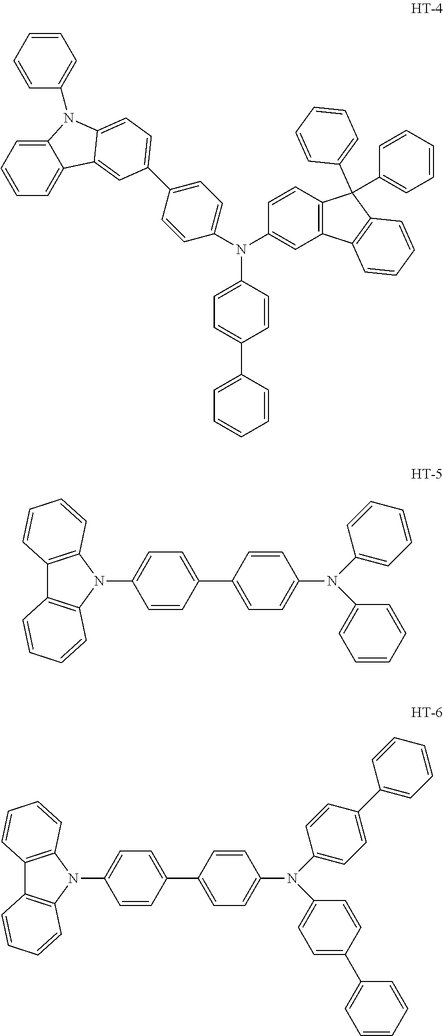 [SMILES: c1ccc(-c2ccc(N(c3ccc(-c4ccc5c(c4)c4ccccc4n5-c4ccccc4)cc3)c3ccc4c(c3)-c3ccccc3C4(c3ccccc3)c3ccccc3)cc2)cc1 ; c1ccc(N(c2ccccc2)c2ccc(-c3ccc(-n4c5ccccc5c5ccccc54)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

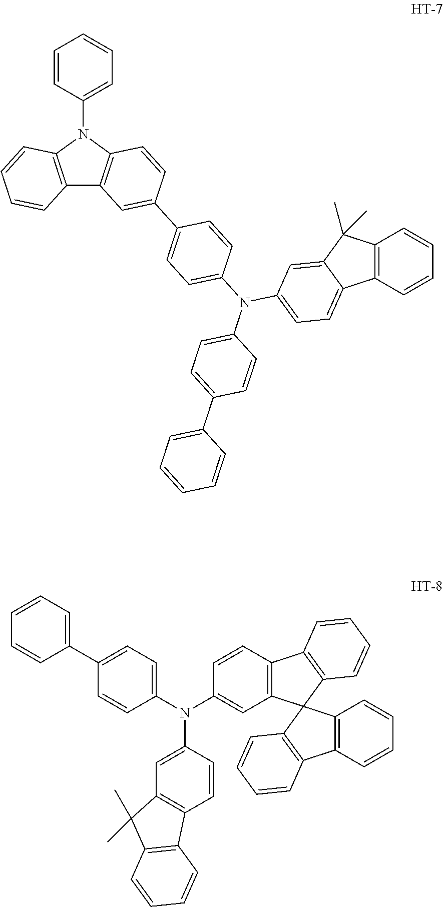 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21]

For example, the hole transfer region 120 may include m-MTDATA (4,4′,4″-[tris(3-methylphenyl)phenylamino]triphenylamine), TDATA (4,4′4″-tris(N,N-diphenylamino)triphenylamine), 2-TNATA (4,4′,4″-tris[N(2-naphthyl)-N-phenylamino]-triphenylamine), NPB (N,N′-di(naphthalene-1-yl)-N,N′-diphenyl-benzidine), NPD (N,N′-Bis(naphthalen-1-yl)-N,N′-bis(phenyl)-2,2′-dimethylbenzidine), TPD (N,N′-bis(3-methylphenyl)-N,N′-diphenyl-[1,1′-biphenyl]-4,4′-diamine), Spiro-TPD, Spiro-NPB, DNTPD (N¹,N^{1′}—([1,1′-biphenyl]-4,4′-diyl)bis(N¹-phenyl-N⁴,N⁴-di-m-tolylbenzene-1,4-diamine), TAPC (4,4′-cyclohexylidene bis[N,N-bis(4-methylphenyl)benzenamine]), HMTPD(4,4′-bis[N,N′-(3-tolyl)amino]-3,3′-dimethylbiphenyl), TCTA (4,4′,4″-tris(N-carbazolyl)triphenylamine), PANI/DBSA (polyaniline/dodecylbenzenesulfonic acid), PEDOT/PSS (poly(3,4-ethylenedioxythiophene)/poly(4-styrenesulfonate)), PANI/CSA (polyaniline/camphor sulfonic acid), PANI/PSS (polyaniline/poly(4-styrenesulfonate)), a phthalocyanine compound, a carbazole compound (N-phenylcarbazole, polyvinylcarbazole, CzSi (9-(4-tert-butylphenyl)-3,6-bis(triphenylsilyl)-9H-carbazole), CCP (9-phenyl-9H-3,9′-bicarbazole), mDCP (1,3-bis(1,8-dimethyl-9H-carbazol-9-yl)benzene), etc.), a fluorene compound, or the like. The hole transfer region 120 may include one of the hole transfer materials described above, or a combination thereof.

The hole transfer materials described above may be included in at least one of the hole injection layer 122, the hole transport layer 124, and the electron blocking layer 126.

The hole transfer region 120 may further include a charge generating material. The charge generating material may be a dopant material such as a p-dopant, so that conductivity of the hole transfer region 120 may be improved.

Examples of the dopant material may include a halogenated metal compound such as LiF, NaCl, CsF, RbCl, RbI, CuI, and KI; a quinone derivative such as TCNQ (tetracyanoquinodimethane), F4-TCNQ (2,3,5,6-tetrafluoro-7,7,8,8-tetracyanoquinodimethane), etc.; a cyano-containing compound such as HATCN (dipyrazino[2,3-f: 2′,3′-h]quinoxaline-2,3,6,7,10,11-hexacarbonitrile), NDP9 (4-[[2,3-bis[cyano-(4-cyano-2,3,5,6-tetrafluorophenyl)methylidene]cyclopropylidene]-cyanomethyl]-2,3,5,6-tetrafluorobenzonitrile), etc.; a tungsten (W) oxide; a molybdenum (Mo) oxide; etc. The hole transfer region 120 may include one of the dopant materials described above, or a combination thereof.

A thickness of the hole transfer region 120 may be in a range of about 100 Å to about 10,000 Å. For example, the thickness of the hole transfer region 120 may be in a range of about 100 Å to about 1,500 Å.

When the hole transfer region 120 includes the hole injection layer 122 or the hole transport layer 124, a thickness of the hole injection layer 122 may be in a range from about 100 Å to about 9,000 Å, from about 100 Å to about 3,000 Å, or from about 100 Å to about 1,000 Å. A thickness of the hole transport layer 124 may be in a range from 50 Å to about 2,000 Å, from about 100 Å to about 1,500 Å, from about 100 Å to about 1,000 Å, or from about 100 Å to about 600 Å.

In the thickness ranges described above, hole transfer properties may be enhanced even at a low voltage operation, and a life-span of the device may be further improved.

Each layer of the hole transfer region 120 may be formed by a process such as a thermal evaporation deposition, a vacuum deposition, a spin coating, an inkjet printing, a laser printing, a casting, a laser thermal transfer, etc.

The electron transfer region 140 may be disposed between the second electrode 150 and the emission layer 130. The electron transfer region 140 may have a single-layered, or a multi-layered structure including different materials.

The electron transfer region 140 may include an electron injection layer, an electron transport layer, and/or a hole blocking layer, and may further include an auxiliary emission layer.

In some embodiments, as illustrated in FIG. 2, the electron transfer region 140 may include an electron injection layer 142 and an electron transport layer 144, stacked from the second electrode 150 to the emission layer 130.

In some embodiments, as illustrated in FIG. 3, the electron transfer region 140 may include an electron injection layer 142, an electron transport layer 144, and a hole blocking layer 146, sequentially stacked from the second electrode 150. The hole blocking layer 146 may block or suppress a hole transfer from the hole transfer region 120. Accordingly, emission energy and luminescence efficiency in the emission layer 130 may be further improved.

For example, the electron transfer region 140 may include a compound represented by Chemical Formula ET.

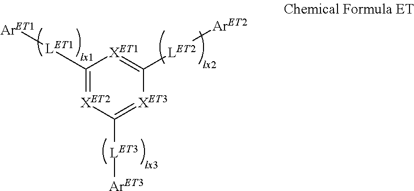

In Chemical Formula ET, at least one of X^{ET1} to X^{ET3} may be N; and the remainder of X^{ET1} to X^{ET3} may each independently be C(R^{ET}). R^{ET} may be a hydrogen atom, a deuterium atom, a substituted or unsubstituted C₁-C₂₀ alkyl group, a substituted or unsubstituted C₆-C₆₀ aryl group, or a substituted or unsubstituted C₂-C₆₀ heteroaryl group.

When one of X^{ET1} to X^{ET3} is N, the compound represented by Chemical Formula ET may include a pyridine group. When two of X^{ET1} to X^{ET3} are N, the compound represented by Chemical Formula ET may include a pyrimidine group. When X^{ET1} to X^{ET3} are each N, the compound represented by Chemical Formula ET may include a triazine group.

In Chemical Formula ET, lx1 to lx3 may each independently be an integer from 0 to 10. L^{ET1} to L^{ET3} may each independently be a direct linkage, a substituted or unsubstituted C₆-C₃₀ arylene group, or a substituted or unsubstituted C₂-C₃₀ heteroarylene group.

When lx1, lx2 or lx3 is 2 or more, two or more of each of L^{ET1} L^{ET2}, or L^{ET3}, respectively, may be linked together, e.g., by carbon atoms of each aryl ring (e.g., sp2 carbons), to form a substituted or unsubstituted C₆-C₃₀ arylene group, or a substituted or unsubstituted C₂-C₃₀ heteroarylene group.

In Chemical Formula ET, Ar^{ET1} to Ar^{ET3} may each independently be a hydrogen atom, a deuterium atom, a substituted or unsubstituted C₁-C₂₀ alkyl group, a substituted or unsubstituted C₆-C₃₀ aryl group, or a substituted or unsubstituted C₂-C₃₀ heteroaryl group. For example, Ar^{ET1} to Ar^{ET3} may each independently be a substituted or unsubstituted phenyl group, a substituted or unsubstituted carbazole group, a substituted or unsubstituted fluorene group, or a substituted or unsubstituted silyl group. The silyl group may be represented by —Si(R^{sa})(R^{sb})(R^{sc}), as explained above.

Non-limiting examples of the electron transfer material included in the electron transfer region 140 are as follows.

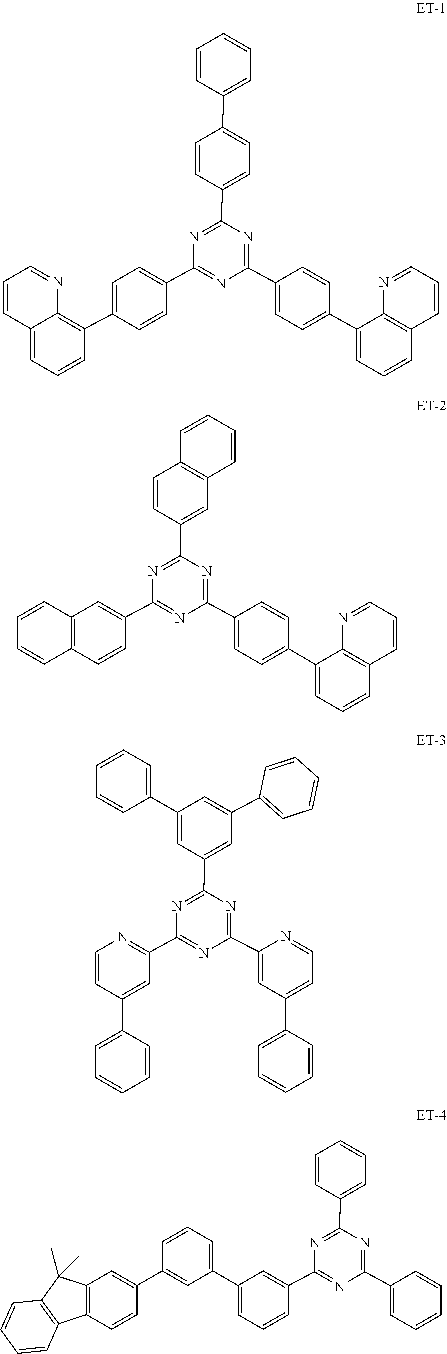 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; c1ccc(-c2cc(-c3ccccc3)cc(-c3nc(-c4cc(-c5ccccc5)ccn4)nc(-c4cc(-c5ccccc5)ccn4)n3)c2)cc1 ; CC1(C)c2ccccc2-c2ccc(-c3cccc(-c4cccc(-c5nc(-c6ccccc6)nc(-c6ccccc6)n5)c4)c3)cc21]

For example, the electron transfer region 140 may include an anthracene compound, Alq₃ (tris(8-hydroxyquinolinato)aluminum), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, TPBi (1,3,5-Tri(1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (Bis(2-methyl-8-quinolinolato-N1,O8)-(1,1′-biphenyl-4-olato)aluminum), Bebg₂ (beryllium bis(benzoquinolin-10-olate)), ADN (9,10-di(naphthalene-2-yl)anthracene), BmPyPhB (1,3-bis[3,5-di(pyridin-3-yl)phenyl]benzene), TSPO1 (diphenyl(4-(triphenylsilyl)phenyl)phosphine oxide), or the like. The electron transfer region 140 may include one of the electron transfer materials described above, or a combination thereof.

The above-mentioned material may be included in at least one of the electron injection layer 142, the electron transport layer 144, and the hole blocking layer 146.

The electron transfer region 140 may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof. In some embodiments, the above-mentioned material may be included electron injection layer 142.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include an oxide, a halide (e.g., a fluoride, a chloride, a bromide, an iodide, etc.), a telluride, or a combination thereof of the alkali metal, the alkaline earth metal, and the rare earth metal, respectively.

The alkali metal complex, the alkaline earth metal complex, and the rare earth metal complex may include a metal ion such as an alkali metal ion, an alkaline earth metal ion or a rare earth metal ion, and a ligand bonded to the metal ion. The ligand may include, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or a combination thereof.

A thickness of the electron transfer region 140 may be in a range from about 100 Å to about 1,000 Å, e.g., from about 150 Å to about 500 Å.

When the electron transfer region 140 includes an electron injection layer 142 or an electron transport layer 144, a thickness of the electron injection layer 142 may be in a range from about 1 Å to about 100 Å, from 1 Å to about 90 Å or from about 5 Å to about 50 Å, and a thickness of the electron transport layer 144 may be in a range from 10 Å to about 900 Å, from about 10 Å to about 500 Å or from about 100 Å to about 400 Å.

Within any of the thickness ranges described above, electron injection and electron transport properties may be further improved without an excessive increase in driving voltage, and stability of the electron transfer region 140 may be improved.

Each layer of the electron transfer region 140 may be formed by a process such as a thermal evaporation deposition, a vacuum deposition, a spin coating, an inkjet printing, a laser printing, a casting, a laser thermal transfer, etc.

The light-emitting device ED may further include a capping layer. Light emission efficiency to an outside of the light-emitting device ED may be improved through the capping layer.

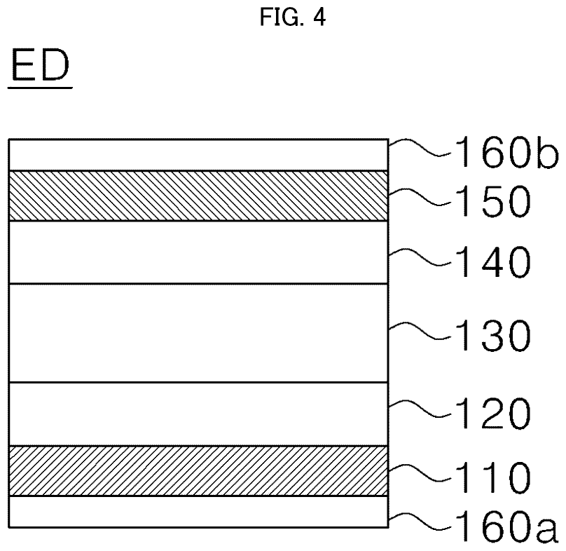

As illustrated in FIG. 4, a second capping layer 160b may be formed on an outer surface of the second electrode 150. In some embodiments, a first capping layer 160a may be formed on an outer surface of the first electrode 110.

A refractive index of the first capping layer 160a and/or the second capping layer 160b may be 1.6 or more. For example, the refractive index of the first capping layer 160a and/or the second capping layer 160b may be 1.6 or more, 1.8 or more, or 2.0 or more for a light in a wavelength range of 550 nm to 660 nm.

The first capping layer 160a and the second capping layer 160b may each be formed as an organic capping layer including an organic material, an inorganic capping layer including an inorganic material, or an organic-inorganic hybrid capping layer including both the organic and inorganic materials.

The first capping layer 160a and/or the second capping layer 160b may each have a single-layered structure or a multi-layered structure including different materials.

In some embodiments, the first capping layer 160a and/or the second capping layer 160b may each independently include a carbocyclic compound, a heterocyclic compound, an amine group-containing compound, a porphine derivative, a phthalocyanine derivative, a naphthalocyanine derivative, an alkaline metal complex, an alkaline earth metal complex, etc. The first capping layer 160a and the second capping layer 160b may each independently include one of the aforementioned materials, or a combination thereof.

In some embodiments, the first capping layer 160a and/or the second capping layer 160b may each independently include an amine group-containing compound.

In a non-limiting example, the first capping layer 160a and/or the second capping layer 160b may include the compound HT-7, the compound HT-8, or the like.

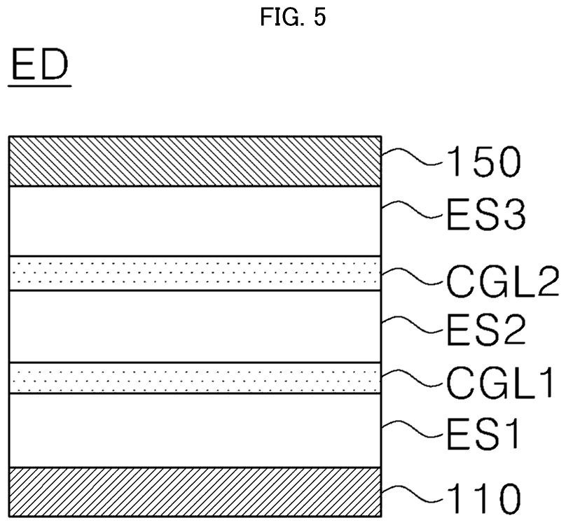

Referring to FIG. 5, the light-emitting device ED may include a plurality of light-emitting structures (e.g., the light-emitting structures ES1, ES2 and ES3). The light-emitting structures ES1, ES2, and ES3 may each include a stacked structure of the hole transfer region 120, the emission layer 130, and the electron transfer region 140, as described with reference to FIGS. 1 to 4. In some embodiments, the light-emitting device ED of FIG. 5 may be a light-emitting device having a tandem structure.

Charge generation layers CGL1 and CGL2 may each be disposed between adjacent structures among the light-emitting structures ES1, ES2 and ES3. Charge generation layers CGL1 and CGL2 may each independently include a p-type charge generation layer and/or an n-type charge generation layer.

The p-type charge generation layer may include a hole transport host compound, such as NPB. For example, the p-type charge generation layer may include a compound represented by Chemical Formula HT as described above. The p-type charge generation layer may further include a p-dopant, such as TCNQ.

In some embodiments, the n-type charge generation layer may include at least one of an alkali metal, an alkaline earth metal, a lanthanide metal, a rare earth metal, a transition metal, a post-transition metal, or an alloy thereof.

The n-type charge generation layer may further include, e.g., a metal complex, and the metal complex may include the above-described metal and at least one organic ligand. The organic ligand may include, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxydiphenyloxadiazole, hydroxydiphenylthiadiazole, hydroxyphenylpyridine, hydroxyphenylbenzimidazole, hydroxyphenylbenzothiazole, bipyridine, phenanthroline, cyclopentadiene, or the like.

The n-type charge generation layer may further include an electron transport host compound. For example, the n-type charge generation layer may include a compound represented by Chemical Formula ET as described above. In some embodiments, the n-type charge generation layer may include a phenanthroline-based compound.

For example, a thickness of the n-type charge generation layer and a thickness of the p-type charge generation layer may each independently be in a range from 20 Å to 1000 Å, from 20 Å to 700 Å, or from 30 Å to 500 Å.

The charge generation layers CGL1 and CGL2 may include a first charge generation layer CGL1 disposed between the first light-emitting structure ES1 and the second light-emitting structure ES2, and a second charge generation layer CGL2 disposed between the second light-emitting structure ES2 and the third-light emitting structure ES3.

In some embodiments, the first light-emitting structure ES1, the first charge generation layer CGL1, the second light-emitting structure ES2, the second charge generation layer CGL2, the third light-emitting structure ES3, and the second electrode 150 may be sequentially stacked from a top surface of the first electrode 110.

Colors emitted from the first light-emitting structure ES1, the second light-emitting structure ES2 and the third light-emitting structure ES3 may be the same as or different from each other. In some embodiments, the first light-emitting structure ES1, the second light-emitting structure ES2 and the third light-emitting structure ES3 may include a red light-emitting layer, a green light-emitting layer and a blue light-emitting layer, respectively, and a white light-emitting structure may be implemented through the tandem structure, but is not limited thereto.

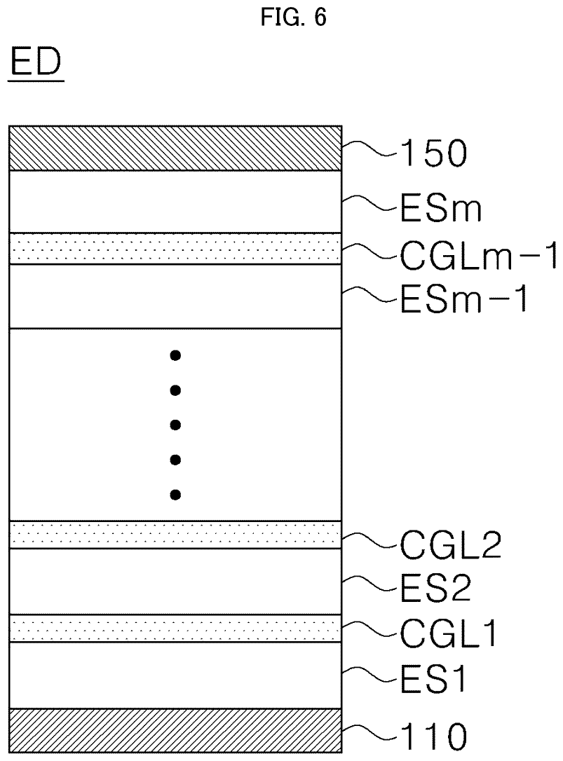

In FIG. 5, the 3-stack tandem structure in which three light-emitting structures are stacked is illustrated as an example, but the tandem structure of the light-emitting device of the present disclosure is not limited to the structure illustrated in FIG. 5. For example, 2-stack structure, or a 4-stack structure, a 5-stack structure, or more stacked structure as will be described with reference FIG. 6 may also be implemented.

Referring to FIG. 6, as described with reference to FIG. 5, a tandem structure in which the light-emitting structure and the charge generation layer are alternately and repeatedly stacked may be disposed between the first electrode 110 and the second electrode 150.

In some embodiments, first to m^{th} light-emitting structures ES1 to ESm may be sequentially stacked from the top surface of the first electrode 110 with the charge generation layer interposed therebetween. The charge generation layer may include a first charge generation layer CGL1 to an (m−1)th charge generation layer CGLm−1 sequentially stacked from the top surface of the first electrode 110.

As illustrated in FIG. 6, the first light-emitting structure ES1, the first charge generation layer CGL1, the second light-emitting structure ES2, the second charge generation layer CGL2, . . . , an (m−1)th light-emitting structure ESm−1, an (m−1)th charge generation layer CGLm−1, an mth light-emitting structure ESm, and the second electrode 150 may be sequentially stacked from the top surface of the first electrode 110.

In some embodiments, m is 4, and the intermediate layer ITL of the light-emitting device may have a 4-stack tandem structure, and may include first to fourth light-emitting structures ES1, ES2, ES3 and ES4, and first to third charge generation layers CGL1, CGL2 and CGL3. Colors of light generated from the first to fourth light-emitting structures ES1, ES2, ES3 and ES4 may be the same as or different from each other.

In some embodiments, the first to fourth light emitting structures ES1, ES2, ES3 and ES4 may include at least one blue light-emitting structure and at least one green-light emitting structure. In a non-limiting example, the first to third light emitting structures ES1, ES2 and ES3 may correspond to the blue light-emitting structure, and the fourth light emitting structure ES4 may correspond to the green-light emitting structure.

In some embodiments, m is 5, and the intermediate layer ITL of the light-emitting device may have a 5-stack tandem structure, and may include first to fifth light-emitting structures ES1, ES2, ES3, ES4 and ES5, and first to fourth charge generation layers CGL1, CGL2, CGL3 and CGL4. Colors of light generated from the first to fifth light-emitting structures ES1, ES2, ES3, ES4, and ES5 may be the same as or different from each other.

In some embodiments, the first to fifth light-emitting structures ES1, ES2, ES3, ES4 and ES5 may include at least one blue light-emitting structure and at least one green light-emitting structure. In a non-limiting example, the first to fifth light-emitting structures ES1, ES2, ES3, ES4 and ES5 may include three blue light-emitting structures and two green light-emitting structures. For example, the first, third and fifth light-emitting structures ES1, ES3 and ES5 may correspond to the blue light-emitting structure, and the second and fourth light-emitting structures ES2 and ES4 may correspond to the green light-emitting structure.

### Electronic Device

The above-described light-emitting device ED may be applied to an electronic device and may be provided as a light-emitting portion or a light-emitting unit of the electronic device.

The electronic device may include the light-emitting device ED including the polycyclic compound of Chemical Formula 1 described above, thereby providing improved color properties, luminous efficiency, and life-span properties.

The electronic device may further include, e.g., a functional layer disposed on the light-emitting device, and the functional layer may include a sensor layer, a polarizing layer, a color conversion layer, a color filter layer, or a combination of at least two thereof.

Examples of an electronic device may include a display device, a billboard, a signboard, a light source, a lighting device, a personal computer such as a laptop computer or a desktop computer, a mobile phone, an electronic book, an electronic dictionary, an electronic notebook, a health-care device including a diagnostic device and various sensors, various display parts for transportation means (automobile, aircraft, ship, train, etc.).

In some embodiments, the light-emitting device ED may be applied to an organic light emitting diode (OLED) display device or a quantum dot (QD)-OLED display device.

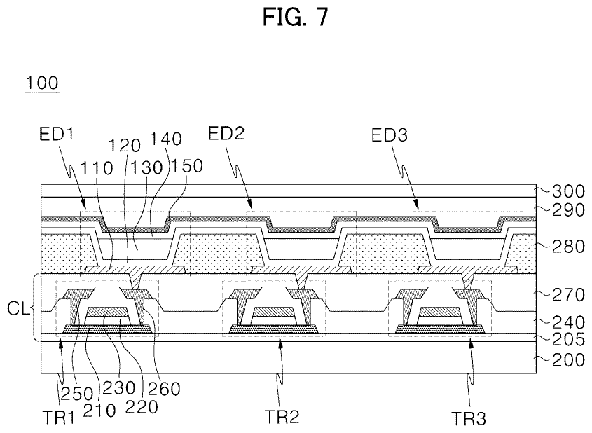

FIG. 7 is a schematic cross-sectional view illustrating a display device in accordance with some embodiments.

Referring to FIG. 7, the display device may include a circuit layer CL disposed on a base substrate 200, and light-emitting devices ED1, ED2 and ED3 disposed on the circuit layer CL.

The base substrate 200 may serve as a supporting substrate or as a back-plane substrate of a display device. The base substrate 200 may be a glass substrate or a plastic substrate.

In some embodiments, the base substrate 200 may include a polymer material having transparent and flexible properties. When the base substrate 200 includes a polymer material, the base substrate 200 may be used in a transparent flexible display device. For example, the base substrate 200 may include a polymer material such as polyimide, polysiloxane, an epoxy resin, an acrylic resin, polyester, etc. In some embodiments, the base substrate 200 may include polyimide.

The circuit layer CL may include transistors TR1, TR2 and TR3. The circuit layer CL may include wiring layers and insulating layers that form a thin film transistor array (TFT-Array).

The circuit layer CL may further include a buffer layer 205 on a top surface of the base substrate 200. The buffer layer 205 may block the penetration of moisture through the base substrate 200, and may also block the diffusion of impurities between the base substrate 200 and the structures formed thereon.

The buffer layer 205 may include, e.g., silicon oxide, silicon nitride, or silicon oxynitride. The buffer layer 205 may include one of the aforementioned materials, or a combination thereof. In some embodiments, the buffer layer 205 may have a stacked structure that includes a silicon oxide layer and a silicon nitride layer.

The transistors TR1, TR2 and TR3 may be disposed on the buffer layer 205. A first transistor TR1, a second transistor TR2 and a third transistor TR3 may be electrically connected to a first light-emitting device ED1, a second light-emitting device ED2 and a third light-emitting device ED3, respectively.

The transistors TR1, TR2 and TR3 may each include an active layer 210, a gate insulation layer 220, and a gate electrode 230.

The active layer 210 may be disposed on the buffer layer 205, and may be patterned for each pixel. The active layer 210 may include a silicon compound such as amorphous silicon or polysilicon. A p-type dopant or an n-type dopant may be doped in a region of the active layer 210, and the active layer 210 may include a source region, a drain region, and a channel region.

The active layer 210 may include an oxide semiconductor, such as indium gallium zinc oxide (IGZO), zinc tin oxide (ZTO), or ITZO.

The gate insulation layer 220 may be formed on the active layer 210, and the gate electrode 230 may be stacked on the gate insulation layer 220. As illustrated in FIG. 7, the gate insulation layer 220 may be patterned to partially cover each active layer 210. Alternatively, the gate insulation layer 220 may extend continuously over multiple pixels or light-emitting regions, and may be provided as a common layer for the first, second, and third transistors TR1, TR2 and TR3.

The gate electrode 230 may overlap the channel region of the active layer 210 in a thickness direction.

An insulating interlayer 240 may be formed on the active layer 210 to cover the gate electrode 230 and the gate insulation layer 220. Connection electrodes 250 and 260 which may be in contact with or electrically connected to the active layer 210 may each be disposed on the insulating interlayer 240.

The connection electrodes 250 and 260 may extend through the insulating interlayer 240 to be in contact with or electrically connected to the active layer 210. When the gate insulation layer 220 is provided as a common layer for multiple light-emitting regions, the connection electrodes 250 and 260 may also extend through the gate insulation layer 220.

The connection electrodes 250 and 260 may include a source electrode 250 that may be in contact with or connected to the source region of the active layer 210, and a drain electrode 260 that may be in contact with or connected to the drain region of the active layer 210.

The gate insulation layer 220 and the insulating interlayer 240 may each independently include silicon oxide, silicon nitride, or silicon oxynitride, and may each have a stacked structure that includes a silicon oxide layer and a silicon nitride layer.

The gate electrode 230 and the connection electrodes 250 and 260 may include a metal such as Ag, Mg, Al, W, Cu, Ni, Cr, Mo, Ti, Pt, Ta, Nd, Sc, an alloy thereof, or a nitride thereof.

A via insulation layer 270 may be formed on the insulating interlayer 240 to cover the connection electrodes 250 and 260.

The via insulation layer 270 may accommodate a via structure electrically connecting the first electrode 110 and the drain electrode 260. The via insulation layer 270 may serve as a planarization layer of the circuit layer CL. In some embodiments, the via insulation layer 270 may include an organic material such as polyimide, an epoxy resin, an acrylic resin, polyester, etc.

The light-emitting devices ED1, ED2 and ED3 may be disposed on the via insulation layer 270. For example, as described with reference to FIGS. 1 to 4, the light-emitting devices ED1, ED2 and ED3 may include the first electrode 110, the hole transfer region 120, the emission layer 130, the electron transfer region 140, and the second electrode 150 which are sequentially stacked from the via insulation layer 270.

The first electrode 110 may be electrically connected to the transistors TR1, TR2 and TR3 or the connection electrodes 250 and 260 in the circuit layer CL through the via structure. As illustrated in FIG. 7, the first electrode 110 may be in contact with or connected to the drain electrode 260 to serve as a pixel electrode patterned for each light-emitting region or pixel.

A pixel defining layer 280 may be formed on the via insulation layer 270 to define each light-emitting region or pixel. A blue light-emitting region, a red light-emitting region, and a green light-emitting region may be separated and defined by the pixel defining layer 280, and the light-emitting devices ED1, ED2, and ED3 may respectively correspond to a blue light-emitting device, a red light-emitting device, and a green light-emitting device.

The pixel defining layer 280 may partially cover the first electrode 110 of each light-emitting region.

As illustrated in FIG. 7, the hole transfer region 120 and the electron transfer region 140 may each be provided as a common layer that continuously extends over the pixel defining layer 280 and the first electrodes 110. The emission layer 130 may be formed within each light emitting-region or pixel, and may be separated by the pixel defining layer 280.

In some embodiments, the emission layer 130 may also be provided as a common layer that continuously extends over the light emitting-regions or pixels. In some embodiments, the hole transfer region 120, the emission layer 130, and the electron transfer region 140 may each be patterned and separately formed for each light-emitting region or pixel.

The second electrode 150 may be provided as a common electrode that continuously extends over the light-emitting regions or the pixels.

An encapsulation layer 290 may be disposed on the pixel defining layer 280 and the light-emitting devices ED1, ED2 and ED3 to protect the light-emitting devices ED1, ED2 and ED3 from moisture and/or oxygen. The encapsulation layer 290 may be a thin film encapsulation (TFE) having a single-layered structure or multi-layered structure.

The encapsulation layer 290 may include an inorganic layer that includes silicon nitride (SiNₓ), silicon oxide (SiOₓ), indium tin oxide, indium zinc oxide, or any combination thereof; an organic layer that includes polyethylene terephthalate, polyethylene naphthalate, polycarbonate, polyimide, polyethylene sulfonate, polyoxymethylene, polyarylate, hexamethyldisiloxane, an acrylic resin (e.g., polymethylmethacrylate, polyacrylic acid, etc.), an epoxy resin (e.g., an aliphatic glycidyl ether (AGE)) or any combination thereof; or a combination of the inorganic layer and the organic layer.

The display device may further include a functional layer 300 disposed on the encapsulation layer 290. The functional layer 300 may include a sensor layer such as a touch sensor layer, an optical layer such as a polarizing layer, a color conversion layer, a color filter layer, a window film, or any combination thereof.

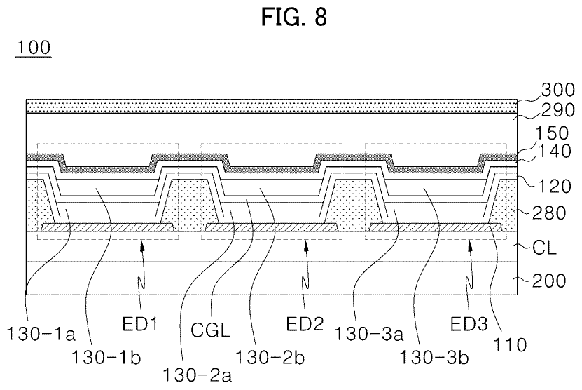

FIG. 8 is a schematic cross-sectional view illustrating a display device in accordance with some embodiments.

Referring to FIG. 8, each of the light-emitting devices ED1, ED2 and ED3 may have a tandem structure, e.g., a 2-stack tandem structure.

In some embodiments, the hole transfer region 120 and the electron transfer region 140 may be continuously and commonly formed and included in an intermediate layer of each light-emitting structure. Additionally, a charge generation layer CGL may continuously extend across a plurality of pixels and may be commonly included in the intermediate layer of each light-emitting structure.

The first light-emitting device ED1 may include a first lower emission layer 130-1a disposed between the hole transfer region 120 and the charge generation layer CGL, and a first upper emission layer 130-1b disposed between the charge generation layer CGL and the electron transfer region 140.

The second light-emitting device ED2 may include a second lower emission layer 130-2a disposed between the hole transfer region 120 and the charge generation layer CGL, and a second upper emission layer 130-2b disposed between the charge generation layer CGL and the electron transfer region 140.

The third light-emitting device ED3 may include a third lower emission layer 130-3a disposed between the hole transfer region 120 and the charge generation layer CGL, and a third upper emission layer 130-3b disposed between the charge generation layer CGL and the electron transfer region 140.

The lower and upper emission layers included in each light-emitting structure may generate light of the same color. In some embodiments, each of the first lower emission layer 130-1a and the first upper emission layer 130-1b included in the first light-emitting device ED1 may correspond to a red emission layer. Each of the second lower emission layer 130-2a and the second upper emission layer 130-2b included in the second light-emitting device ED2 may correspond to a green emission layer. Each of the third lower emission layer 130-3a and the third upper emission layer 130-3b included in the third light-emitting device ED3 may correspond to a blue emission layer.

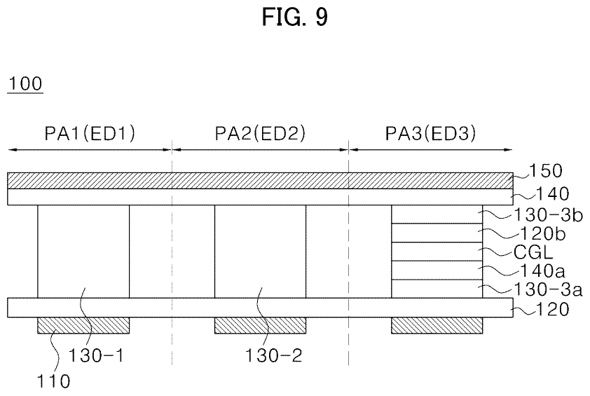

FIG. 9 is a schematic cross-sectional view illustrating a stack construction of light-emitting structure in a display device in accordance with some embodiments. For convenience of illustration and description, illustration of the circuit layer, the base substrate, the pixel defining layer, etc., is omitted from FIG. 9, and a shape of each layer or element in the light-emitting structure is briefly shown as a rectangle.

Referring to FIG. 9, at least one of the light-emitting devices ED1, ED2 and ED3 or pixel areas PA1, PA2 and PA3 may have a tandem structure including a plurality of emission layers, and at least one of the remainder may have a single emission layer structure.

In some embodiments, one of the light-emitting devices ED1, ED2 and ED3 or the pixel areas PA1, PA2 and PA3 may have a tandem structure, and the remainder may have a single emission layer structure.

As illustrated in FIG. 9, the first light-emitting device ED1, the second light-emitting device ED2, and the third light-emitting device ED3 may be included in the first pixel area PA1, the second pixel area PA2, and the third pixel area PA3, respectively. In some embodiments, the first pixel area PA1, the second pixel area PA2, and the third pixel area PA3 may correspond to a red pixel area, a green pixel area, and a blue pixel area, respectively.

The hole transfer region 120, the electron transfer region 140, and the second electrode 150 may each be provided as a common layer continuously extending over the first pixel area PA1, the second pixel area PA2, and the third pixel area PA3.

The first-light emitting device ED1 included in the first pixel area PA1 may include a first emission layer 130-1, and the second light-emitting device ED2 included in the second pixel area PA2 may include a second emission layer 130-2. Each of the first emission layer 130-1 and the second emission layer 130-2 may be a single-layered emission layer.

The third light-emitting device ED3 included in the third pixel area PA3 may have, e.g., a 2-stack tandem structure. The third light-emitting device ED3 may include a third lower emission layer 130-3a and a third upper emission layer 130-3b separated with the charge generation layer CGL interposed therebetween. Each of the third lower emission layer 130-3a and the third upper emission layer 130-3b may correspond to a blue emission layer.

A lower electron transfer region 140a may be disposed between the charge generation layer CGL and the third lower emission layer 130-3a. An upper hole transfer region 120b may be disposed between the charge generation layer CGL and the third upper emission layer 130-3b.

Accordingly, a tandem light-emitting structure in which the first electrode 110, the hole transfer region 120, the third lower emission layer 130-3a, the lower electron transfer region 140a, the charge generation layer CGL, the upper hole transfer region 120b, the third upper emission layer 130-3b, the electron transfer region 140, and the second electrode 150 are sequentially stacked may be disposed in the third pixel area PA3.

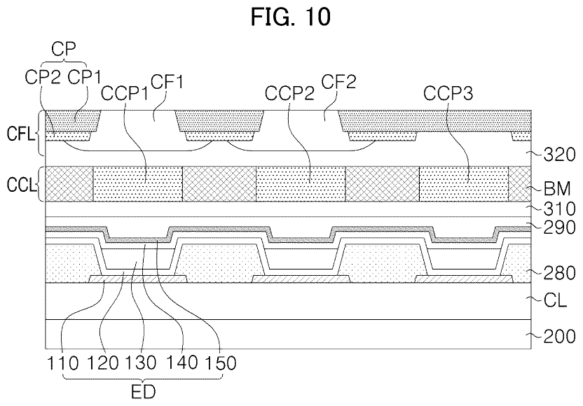

FIG. 10 is a schematic cross-sectional view illustrating a display device in accordance with some embodiments.

FIG. 10 illustrates a display device having a QD-OLED structure according to some embodiments. Detailed descriptions regarding elements and structures that are the same as or substantially similar to those described with reference to FIG. 7 will not be repeated here.

Referring to FIG. 10, the pixel defining layer 280 and the light-emitting device ED may be disposed on the circuit layer CL, as described above with reference to FIG. 7. In some embodiments, each pixel may emit light of the same wavelength region. In some embodiments, each light-emitting device ED may emit a blue light.

In some embodiments, each light-emitting region may include the light-emitting device having the tandem structure, as described above with respect to FIG. 5. In this case, the intermediate layer of each light-emitting device ED may be provided as a common layer that continuously extends over a plurality of the light-emitting regions.

A color control layer CCL may be disposed on the encapsulation layer 290, and the color control layer CCL may include color control portions CCP1, CCP2, and CCP3.

The color control portions CCP1, CCP2 and CCP3 may each include a light transformer such as a quantum dot or a phosphor. In each of the color control portions CCP1, CCP2 and CCP3, the light transformer may convert a wavelength of a provided light and emit a resulting light.

The color control portions CCP1, CCP2 and CCP3 may be separated or spaced apart from each other by a bank BM. The bank BM may substantially overlap the pixel defining layer 280, and the color control portions CCP1, CCP2 and CCP3 may substantially overlap each of the emission layers 130.

The color control layer CCL may include a first color control portion CCP1 including a first quantum dot that converts a first color light provided from the light-emitting device ED into a second color light, a second color control portion CCP2 including a second quantum dot that converts the first color light into a third color light, and a third color control portion CCP3 that transmits the first color light.

In some embodiments, the first color light, the second color light, and the third color light may be a blue light, a red light, and a green light, respectively. The first quantum dot and the second quantum dot may respectively be a red quantum dot and a green quantum dot.

The color control portions CCP1, CCP2 and CCP3 may each further include a scattering material such as inorganic particles. The third color control portion CCP3 may not include quantum dots and may include the scattering material. The scattering material may include TiO₂, ZnO, Al₂O₃, SiO₂, hollow silica, etc. The scattering material may be one of the aforementioned materials or a combination thereof.

The color control portions CCP1, CCP2, and CCP3 may each further include a binder resin that disperses the quantum dot and the scattering material. The binder resin may include an acrylic resin, a urethane resin, a silicone resin, an epoxy resin, etc.

A color filter layer CFL that includes color filters CF1 and CF2 and a light-shielding portion CP may be disposed on the color control layer CCL.

The color filter layer CFL may include a first filter CF1 that transmits the second color light, a second filter CF2 that transmits the third color light, and a third filter that transmits the first color light. For example, the first filter CF1 may be a red filter, the second filter CF2 may be a green filter, and the third filter may be a blue filter.

The color filters CF1 and CF2 may each include a photosensitive binder resin and a colorant including a pigment and/or a dye. The first filter CF1 may include a red pigment or dye, and the second filter CF2 may include a green pigment or dye.

The light-shielding portion CP may be disposed between the color filters. In some embodiments, the light-shielding portion may include a first light-shielding portion CP1 and a second light-shielding portion CP2 that includes colorants of different colors.

In some embodiments, the first light-shielding portion CP1 may include a blue colorant, and the second light-shielding portion CP2 may include a red colorant or a black colorant. In some embodiments, in the blue light-emitting region, a portion of the first light-shielding portion CP1 may be exposed between the second light-shielding portions CP2 may be provided as a blue color filter and, so that an additional color filter (e.g., the third filter) may be omitted.

A first barrier layer 310 may be disposed between the color control layer CCL and the light-emitting device ED (or the encapsulation layer 290). A second barrier layer 320 may be disposed between the color control layer CCL and the color filter layer CFL.

The barrier layers 310 and 320 may each include at least one inorganic layer. For example, the barrier layers 310 and 320 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, etc.

In some embodiments, the barrier layers 310 and 320 may each have a multi-layered structure that further includes an organic layer.

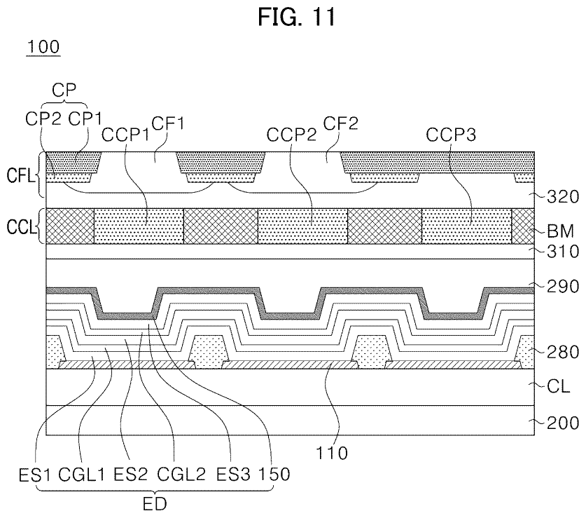

FIG. 11 is a schematic cross-sectional view illustrating a display device in accordance with some embodiments. Detailed descriptions of elements and structures substantially the same as or similar to those described with reference to FIG. 10 are omitted herein.

Referring to FIG. 11, the light-emitting device ED corresponding to the color control portions CCP1, CCP2 and CCP3 may be disposed on the first electrode 110 serving as the pixel electrode, and the light-emitting device ED may have a tandem structure.

In some embodiments, as described with reference to FIG. 5, the first light-emitting structure ES1, the first charge generation layer CGL1, the second light-emitting structure ES2, the second charge generation layer CGL2, and the third light-emitting structure ES3 may be sequentially stacked between the first electrode 110 and the second electrode 150. The first light-emitting structure ES1, the first charge generation layer CGL1, the second light-emitting structure ES2, the second charge generation layer CGL2, and the third light-emitting structure ES3 may be continuously and commonly formed in a plurality of pixel areas or light-emitting regions.

In some embodiments, the first light-emitting structure ES1, the second light-emitting structure ES2, and the third light-emitting structure ES3 may generate different color lights, and the light-emitting device ED may generate a white light. In some embodiments, the first light-emitting structure ES1, the second light-emitting structure ES2, and the third light-emitting structure ES3 may all generate blue lights.

In some embodiments, as described with reference to FIG. 6, the light-emitting device ED may include a tandem structure of 4-stack, 5-stack, or more of the stacked number.

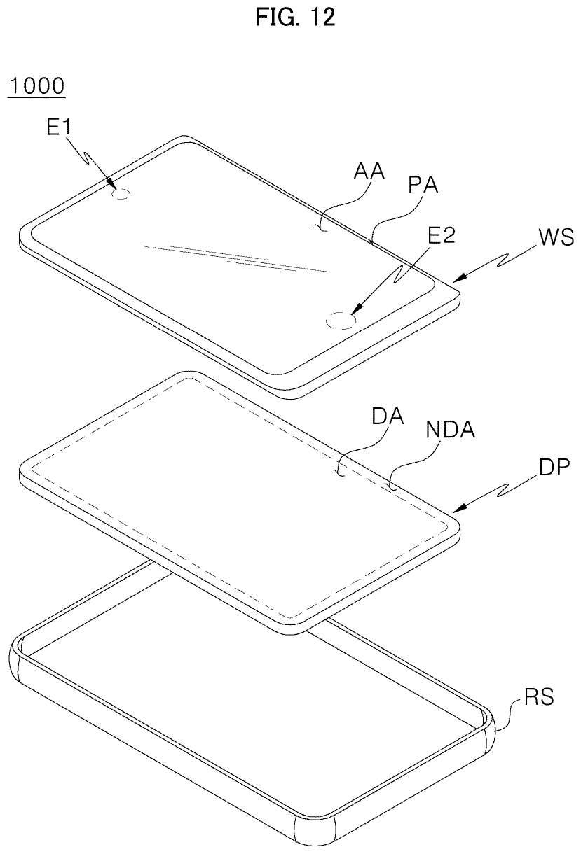

FIG. 12 is a schematic exploded perspective view illustrating an electronic device in accordance with some embodiments.

According to some embodiments, the electronic device may be implemented in the form of a mobile phone (smart phone), a tablet, a PC, or the like, including the above-described display device.

Referring to FIG. 12, the electronic device may include a window structure WS, a display panel DP, and a rear structure RS.

The window structure WS may provide an external display surface recognized by a user, such as a viewing surface of a mobile phone, and may include a transparent material film. For example, the window structure WS may include glass (e.g., ultra-thin glass (UTG), a hard coating film, a plastic film, or the like.

An outer surface of the window structure WS may include an active area AA and a peripheral area PA. The active area AA may provide a surface from which an image of the display device DD is substantially displayed and to which a user's touch/command is input. The peripheral area PA may substantially correspond to a bezel area of the display device.

The display panel DP may include the above-described display device and may have a display area DA and a non-display area NDA. The display area DA of the display panel DP may substantially correspond to or overlap the active area AA of the window structure WS. The non-display area NDA of the display panel DP may substantially correspond to or overlap the peripheral area PA of the window structure WS.

In some embodiments, functional device areas E1 and E2 may be included in the active area AA of the window structure WS. For example, a first functional device area E1 may be included at one end portion of the active area AA and may be implemented, e.g., in the form of a camera hole. The second functional device area E2 may serve as a fingerprint sensing area.

For example, a sensor structure for a touch sensing or a fingerprint sensing may be disposed in the display panel DP or between the window structure WS and the display panel DP.

The rear structure RS may serve as a frame structure or a housing of the display device or the electronic device. A cover panel may be disposed between the rear structure RS and the display panel DP.

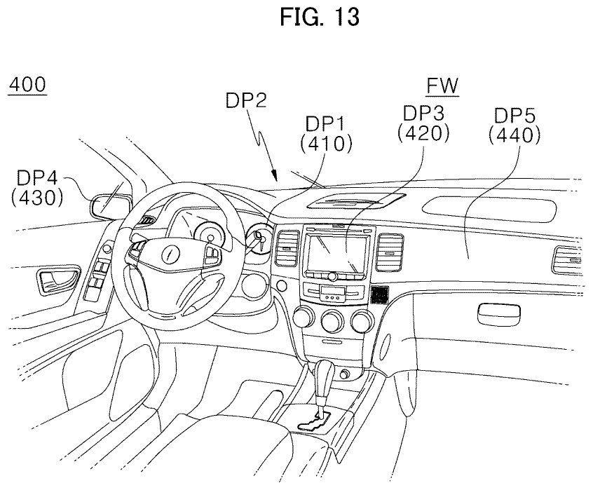

FIG. 13 is a schematic view illustrating an electronic device in accordance with some embodiments.

The electronic device may be installed in, embedded in, attached to, or integrated with a vehicle 400. However, the vehicle 400 is not limited to the embodiment illustrated in FIG. 13 Further examples of the vehicle 400 may include a transportation means such as a three-wheeled or four-wheeled vehicle, a construction machine, a two-wheeled vehicle, a motor vehicle, a bicycle, a train, etc. Other examples of the vehicle 400 may include an electric vehicle, a hybrid vehicle, etc.

Referring to FIG. 13, at least one of first to fifth display devices DP1, DP2, DP3, DP4, and DP5 may be applied to the vehicle 400.

In some embodiments, the first display device DP1 may be disposed in a cluster area 410. Driving information such as a driving distance and speed, and various warning lights may be displayed in the cluster area 410.

The second display device DP2 may be disposed on a front window FW of the vehicle 400. For example, the second display device DP2 may be installed as a head-up display (HUD).

The third display device DP3 may be disposed on a center fascia 420 of the vehicle 400. In the center fascia 420, a button or a switch for controlling an image display or a music player, an air conditioner, a heater, etc., may be displayed, and vehicle information may be displayed thereon.

The fourth display device DP4 may be applied to side mirrors 430 of the vehicle 400. A side mirror 430 may be installed at each of both sides of an exterior of the vehicle 400, and the fourth display device DP4 may be applied to at least one of the side mirrors 430 installed at each of the both sides.

The fifth display device DP5 may be disposed on a passenger seat dashboard 440. Information/image identical to or different from information/image displayed on the cluster area 410 and/or the center fascia 420 may be displayed at the passenger seat dashboard 440.

In some embodiments, the electronic device may include, e.g., a flat panel display, a curved display, a computer monitor, a medical monitor, a television, a billboard, a light for indoor or outdoor lighting, a signal light, a head-up display, a full or partial transparent display, a flexible display, a rollable display, a foldable display, a stretchable display, a laser printer, a phone, a mobile phone, a tablet, a phablet, a personal digital assistant (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

The display device according to some embodiments of the present disclosure may be applied to various electronic devices. The electronic device according to some embodiments include the above-described display device, and may further include a module or device having another additional function in addition to the display device.

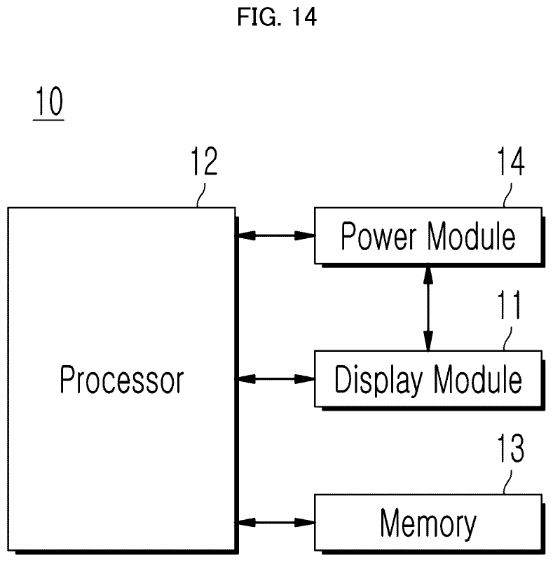

FIG. 14 is a block diagram of an electronic device in accordance with some embodiments.

Referring to FIG. 14, an electronic device 10 according to some embodiments may include a display module 11, a processor 12, a memory 13 and a power module 14.

The processor 12 may include a central processing unit (CPU), an application processor (AP), a graphic processing unit (GPU), a communication processor (CP), an image signal processor (ISP) and/or a controller.

Data information for an operation of the processor 12 or the display module 11 may be stored in the memory 13. When the processor 12 executes an application stored in the memory 13, an image data signal and/or an input control signal may be transmitted to the display module 11, and the display module 11 may process the received signal and output image information through a display screen.

The power module 14 may include a power supply module such as a power adapter or a battery device, and a power conversion module that converts a power supplied by the power supply module to a generate power required for the operation of the electronic device 10.

At least one of components of the electronic device 10 as described above may be included in the display device according to the above-described embodiments. Additionally, some of individual modules functionally included in one module may be included in the display device, and others may be provided separately from the display device. For example, the display module 11 may include the display device, and the processor 12, the memory 13 and the power module 14 may be provided in the form of another device in the electronic device 10 different from the display device.

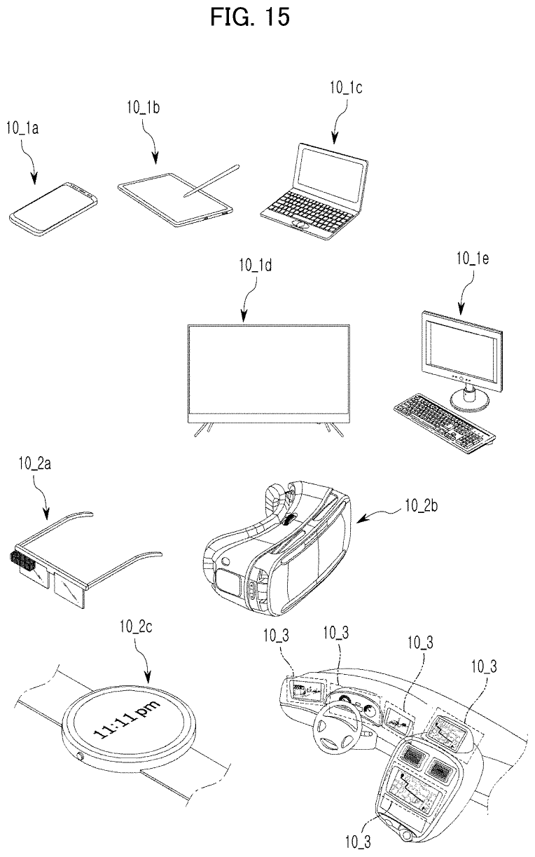

FIG. 15 shows schematic diagrams of an electronic device in accordance with some embodiments.

Referring to FIG. 15, non-limiting examples of various electronic devices to which the display device according to the above-described embodiments is applied include an electronic device for displaying an image such as a smartphone 10_1a, a tablet PC 10_1b, a laptop 10_1c, a TV 10_1d, a desk monitor 10_1e, or the like; a wearable electronic device including a display module such as smart glasses 10_2a, a head mounted display 10_2b, a smart watch 10_2c, or the like; a vehicle electronic device 10_3 including a display module such as a center information display (CID) disposed at a vehicle instrument panel, a center fascia, a dashboard, etc., a room mirror display, a head-up display, or the like. The electronic device may include a virtual reality glass or an augmented reality glass.

Hereinafter, some embodiments will be described in detail with reference to experimental examples including Examples and the Comparative Examples. The Examples are provided to assist in understanding the disclosure, but they are provided as non-limiting examples, and the scope of the disclosure is not limited thereto. It will be clear to those skilled in the art that various changes and modifications to disclose examples can be made within the scope of the disclosure.

### Synthesis Example 1: Synthesis of Compound 1

A compound 1 was synthesized according to a reaction scheme as follows.

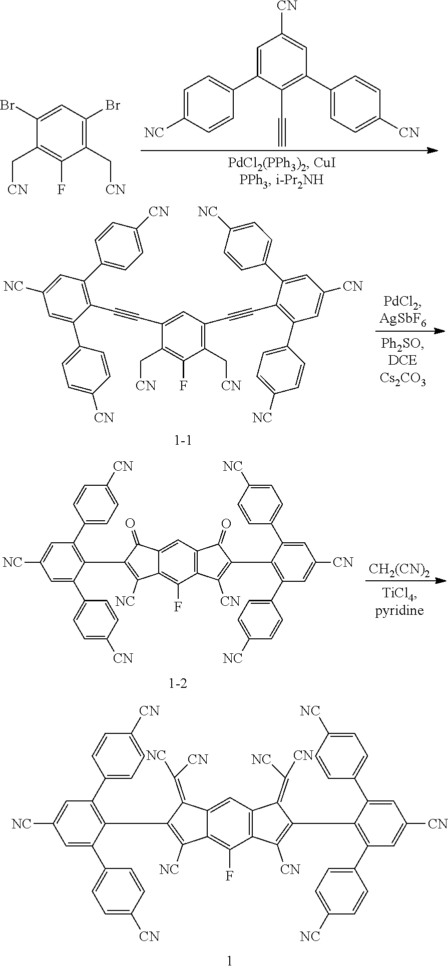

### 1) Synthesis of Intermediate 1-1

Under a nitrogen atmosphere, 2,2′-(4,6-dibromo-2-fluoro-1,3-phenylene)diacetonitrile (1 eq), 2′-ethynyl-[1,1′:3′,1″-terphenyl]-4,4″,5′-tricarbonitrile (2 eq), PdCl₂(PPh₃)₂ (0.05 eq), copper iodide (0.05 eq), triphenylphosphine (0.1 eq), and diisopropylamine (0.2 eq) were dissolved in toluene and stirred at 50° C. for 24 hours. After cooling, the mixture was washed three times with ethyl acetate and water, and the organic layer was separated. The obtained organic layer was dried over MgSO₄ and then dried under reduced pressure. An intermediate 1-1 was obtained by purification using a column chromatography (yield 59%).

### 2) Synthesis of Intermediate 1-2

The intermediate 1-1 (1 eq), PdCl₂ (0.05 eq), silver hexafluoroantimonate (V) (0.1 eq), and diphenylsulfoxide (3 eq) were dissolved in dichloroethylene, stirred at 60° C. for 24 hours, and then Cs₂CO₃ (0.1 eq) was added and stirred for 12 hours. Thereafter, the mixture was extracted with dichloromethane, added to 35% hydrochloric acid, and stirred for 2 hours. After cooling, an organic layer obtained by extraction with dichloromethane and a NH₄Cl aqueous solution was dried under reduced pressure over MgSO₄. An intermediate 1-2 was obtained using a column chromatography (yield 38%).

### 3) Synthesis of Compound 1

The intermediate 1-2 (1 eq), malononitrile (2 eq), and dichloromethane were stirred for 30 minutes in a nitrogen atmosphere. Titanium tetrachloride (3 eq) was slowly injected, pyridine was added, and stirred at room temperature. After cooling, an organic layer obtained by extraction with dichloromethane and a NH₄Cl aqueous solution was dried under reduced pressure over MgSO₄. The compound 1 was obtained using a column chromatography (yield 2.2%).

The obtained compound was confirmed by MS/FAB.

C₆₂H₂₁FN₁₂ cal. 952.20, found 952.21.

### Synthesis Example 2: Synthesis of Compound 36

A compound 36 was synthesized according to a reaction scheme as follows.

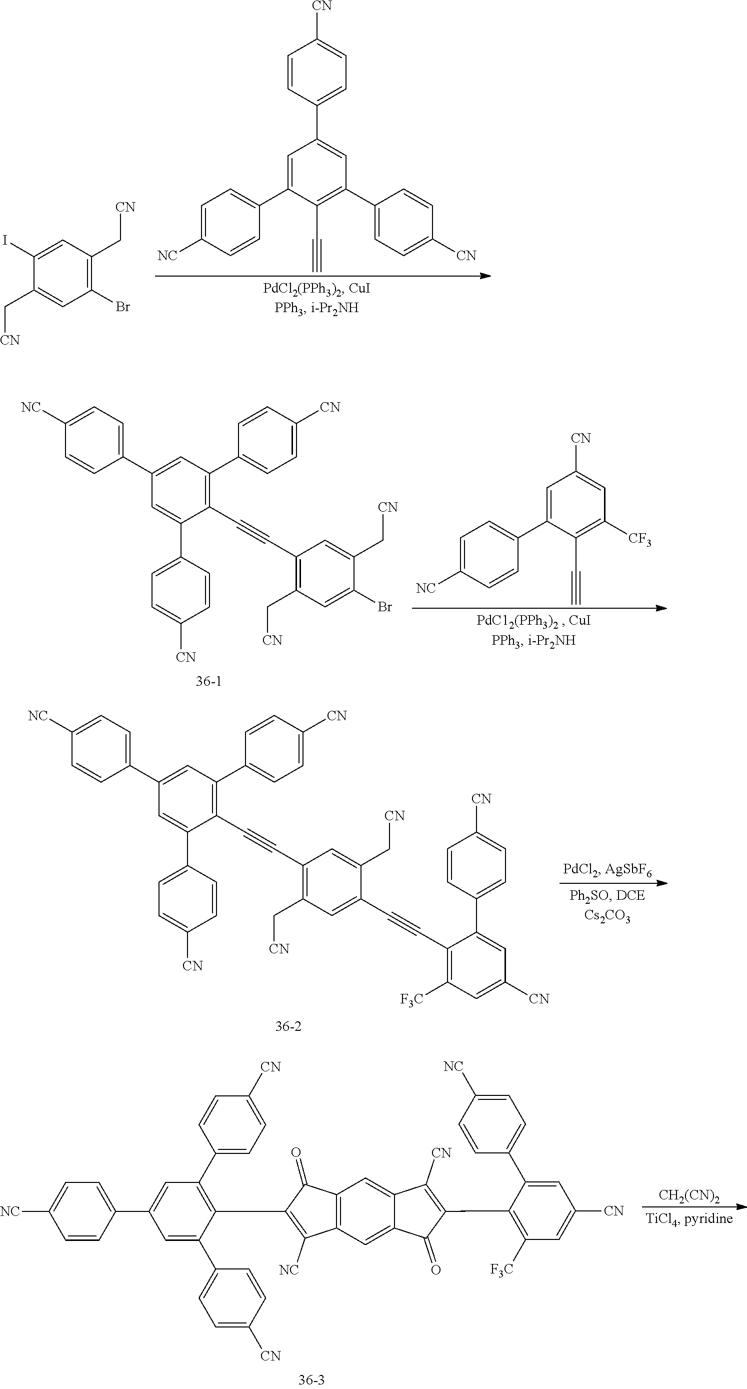

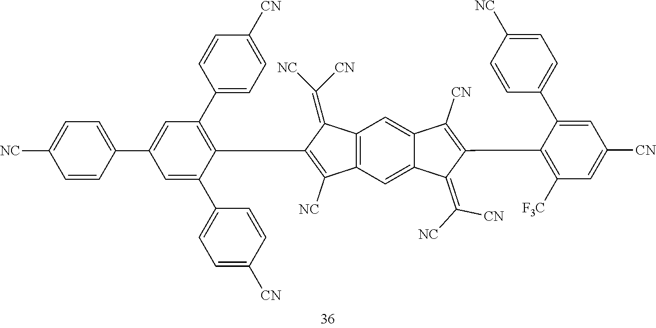 [SMILES: N#CC(C#N)=C1C(c2c(-c3ccc(C#N)cc3)cc(-c3ccc(C#N)cc3)cc2-c2ccc(C#N)cc2)=C(C#N)c2cc3c(cc21)C(C#N)=C(c1c(-c2ccc(C#N)cc2)cc(C#N)cc1C(F)(F)F)C3=C(C#N)C#N]

### 1) Synthesis of Intermediate 36-1

Under a nitrogen atmosphere, 2,2′-(2-bromo-5-iodo-1,4-phenylene)diacetonitrile (1 eq), 5′-(4-cyanophenyl)-2′-ethynyl-[1,1′:3′,1″-terphenyl]-4,4″-dicarbonitrile (1 eq), PdCl₂(PPh₃)₂ (0.05 eq), copper iodide (0.05 eq), triphenylphosphine (0.1 eq), and diisopropylamine (0.2 eq) were dissolved in toluene and stirred at 50° C. for 24 hours. After cooling, the mixture was washed three times with ethyl acetate and water, and the organic layer was separated. The obtained organic layer was dried over MgSO₄, and then dried under reduced pressure. An intermediate 36-1 was obtained by purification using a column chromatography (yield 65%).

### 2) Synthesis of Intermediate 36-2

Under a nitrogen atmosphere, the intermediate 36-1 (1 eq), 6-ethynyl-5-(trifluoromethyl)-[1,1′-biphenyl]-3,4′-dicarbonitrile (1 eq), PdCl₂(PPh₃)₂ (0.05 eq), copper iodide (0.05 eq), triphenylphosphine (0.1 eq), and diisopropylamine (0.2 eq) were dissolved in toluene and stirred at 50° C. for 24 hours. After cooling, the mixture was washed three times with ethyl acetate and water, and an organic layer was separated. The obtained organic layer was dried over MgSO⁴, and then dried under reduced pressure. An intermediate 36-2 was obtained by purification using a column chromatography (yield 58%).

### 3) Synthesis of Intermediate 36-3

The intermediate 36-2 (1 eq), PdCl₂ (0.05 eq), silver hexafluoroantimonate (V) (0.1 eq), and diphenylsulfoxide (3 eq) were dissolved in dichloroethylene. The mixture was stirred at 60° C. for 24 hours, and then Cs₂CO₃ (0.1 eq) was added and stirred for 12 hours. Thereafter, the mixture was extracted with dichloromethane, added to 35% hydrochloric acid, and stirred for 2 hours. After cooling, an organic layer obtained by extracting with dichloromethane and a NH₄Cl aqueous solution was dried under reduced pressure over MgSO₄. An intermediate 36-3 was obtained using a column chromatography (yield 36%).

### 4) Synthesis of Compound 36

The intermediate 36-3 (1 eq), malononitrile (2 eq), and dichloromethane were stirred for 30 minutes under a nitrogen atmosphere. Titanium tetrachloride (3 eq) was slowly added, pyridine was added, and stirred at room temperature. After cooling, an organic layer obtained by extracting it with dichloromethane and a NH₄Cl aqueous solution was dried under reduced pressure with MgSO₄. The compound 36 was obtained using a column chromatography (yield 2.0%).

The obtained compound was confirmed by MS/FAB.

C₆₂H₂₂F₃N₁₁ cal. 977.20, found 977.22.

### Synthesis Example 3: Synthesis of Compound 77

A compound 77 was synthesized according to a reaction scheme as follows.

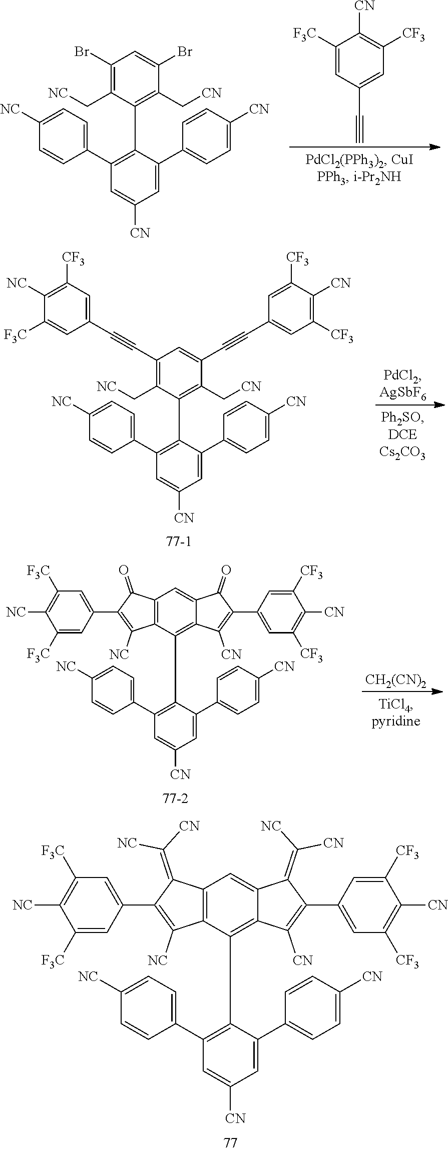

### 1) Synthesis of Intermediate 77-1

2′-(3,5-dibromo-2,6-bis(cyanomethyl)phenyl)-[1,1′:3′,1″-terphenyl]-4,4″,5′-tricarbonitrile (1 eq), 4-ethynyl-2,6-bis(trifluoromethyl)benzonitrile (2 eq), PdCl₂(PPh₃)₂ (0.05 eq), copper iodide (0.05 eq), triphenylphosphine (0.1 eq), and diisopropylamine (0.2 eq) were dissolved in toluene under a nitrogen atmosphere and stirred at 50° C. for 24 hours. After cooling, the mixture was washed three times with ethyl acetate and water, and an organic layer was separated. The obtained organic layer was dried over MgSO₄, and then dried under reduced pressure. An intermediate 77-1 was obtained by purification using a column chromatography (yield 60%).

### 2) Synthesis of Intermediate 77-2

The intermediate 77-1 (1 eq), PdCl₂ (0.05 eq), silver hexafluoroantimonate (V) (0.1 eq), and diphenylsulfoxide (3 eq) were dissolved in dichloroethylene and stirred at 60° C. for 24 hours. Cs₂CO₃ (0.1 eq) was added and stirred for 12 hours. Thereafter, the mixture was extracted with dichloromethane, added to 35% hydrochloric acid, and stirred for 2 hours. After cooling, an organic layer obtained by extracting it with dichloromethane and a NH₄Cl aqueous solution was dried under reduced pressure with MgSO₄. An intermediate 77-2 was obtained by purification using a column chromatography (yield 39%).

### 3) Synthesis of Compound 77

The intermediate 77-2 (1 eq), malononitrile (2 eq), and dichloromethane were stirred for 30 minutes under a nitrogen atmosphere. Titanium tetrachloride (3 eq) was slowly added, pyridine was added, and stirred at room temperature. After cooling, an organic layer obtained by extracting it with dichloromethane and a NH₄Cl aqueous solution was dried under reduced pressure with MgSO₄. The compound 77 was obtained using a column chromatography (yield 2.3%).

The obtained compound was confirmed by MS/FAB.

C₅₉H₁₅F₁₂N₁₁ cal. 1105.13, found 1105.12.

### Synthesis Example 4: Synthesis of Compound 89

A compound 89 was synthesized according to a reaction scheme as follows.

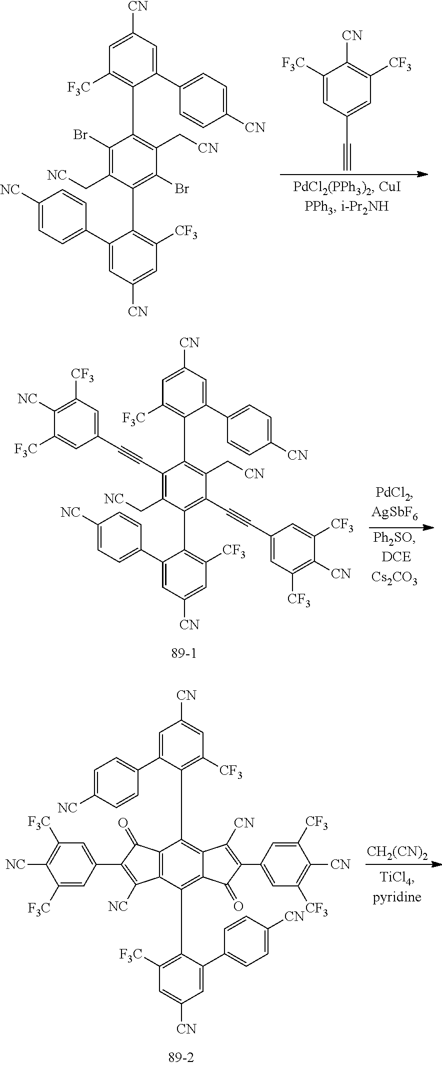

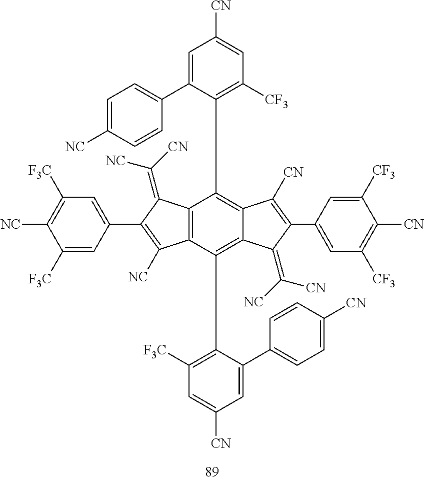 [SMILES: N#CC(C#N)=C1C(c2cc(C(F)(F)F)c(C#N)c(C(F)(F)F)c2)=C(C#N)c2c1c(-c1c(-c3ccc(C#N)cc3)cc(C#N)cc1C(F)(F)F)c1c(c2-c2c(-c3ccc(C#N)cc3)cc(C#N)cc2C(F)(F)F)C(=C(C#N)C#N)C(c2cc(C(F)(F)F)c(C#N)c(C(F)(F)F)c2)=C1C#N]

### 1) Synthesis of Intermediate 89-1

2″,5″-dibromo-3″,6″-bis(cyanomethyl)-3′,6′″-bis(trifluoromethyl)-[1,1′:2′,1″:4″,1′″:2′″,1″″-quinquephenyl]-4,4′″,4″″,5′-tetracarbonitrile (1 eq), 5-ethynyl-2-isocyano-1,3-bis(trifluoromethyl)benzene (2 eq), PdCl₂(PPh₃)₂ (0.05 eq), copper iodide (0.05 eq), triphenylphosphine (0.1 eq), and diisopropylamine (0.2 eq) were dissolved in toluene and stirred at 50° C. for 24 hours. After cooling, the mixture was washed three times with ethyl acetate and water, and an organic layer was separated. The obtained organic layer was dried over MgSO₄, and then dried under reduced pressure. An intermediate 89-1 was obtained by purification using a column chromatography (yield 56%).

### 2) Synthesis of Intermediate 89-2

The intermediate 89-1 (1 eq), PdCl₂ (0.05 eq), silver hexafluoroantimonate (V) (0.1 eq), and diphenylsulfoxide (3 eq) were dissolved in dichloroethylene and stirred at 60° C. for 24 hours. Thereafter, Cs₂CO₃ (0.1 eq) was added and stirred for 12 hours. The mixture was extracted with dichloromethane, added to 35% hydrochloric acid, and stirred for 2 hours. After cooling, an organic layer obtained by extracting it with dichloromethane and a NH₄Cl aqueous solution was dried under reduced pressure with MgSO₄. An intermediate 89-2 was obtained using a column chromatography (yield 37%).

### 3) Synthesis of Compound 89

The intermediate 89-2 (1 eq), malononitrile (2 eq), and dichloromethane were stirred for 30 minutes under a nitrogen atmosphere. Titanium tetrachloride (3 eq) was slowly injected, pyridine was added, and the mixture was stirred at room temperature. After cooling, an organic layer obtained by extracting it with dichloromethane and a NH₄Cl aqueous solution was dried under reduced pressure with MgSO₄. A compound 89 was obtained using a column chromatography (yield 1.7%).

The obtained compound was confirmed by MS/FAB.

C₆₈H₁₆F₁₈N₂ cal. 1342.13, found 1342.12.

### Fabrication of Light-Emitting Device

As the first electrode, a glass substrate (Corning product) on which a 15 Ω/cm² (1200 Å) ITO electrode was formed was cut into a size of 50 mm×50 mm×0.7 mm, and the cut substrate was ultrasonically cleaned for 5 minutes using isopropyl alcohol and pure water. The ultrasonically cleaned substrate was irradiated with an ultraviolet ray for 30 minutes and exposed to ozone, and then mounted on a vacuum deposition device.

Thereafter, a p-dopant compound and HT-7 were vacuum-deposited on the anode in a weight ratio of 3:97 to form a hole injection layer having a thickness of 100 Å. HT-8 was vacuum-deposited on the hole injection layer to form a hole transport layer having a thickness of 1250 Å. PH-8, PH-7 and PD1-9 were vacuum-deposited on the hole transport layer in a weight ratio of 45:45:10 to form an emission layer having a thickness of 300 Å.

ET-4 was vacuum-deposited on the emission layer to form a hole-blocking layer having a thickness of 50 Å. ET-4 and LiQ were vacuum-deposited on the hole-blocking layer in a weight ratio of 5:5 to form an electron transport layer having a thickness of 310 Å.

Yb was vacuum-deposited on the electron transport layer to form an electron injection layer having a thickness of 15 Å. Ag and Mg were vacuum-deposited on the electron injection layer in a weight ratio of 5:5 to form a cathode having a thickness of 1,000 Å.

As the p-dopant compound, compounds of Examples synthesized according to the above-described Synthesis Examples and commercially available compounds of Comparative Examples as shown below were used.

### Compounds of Examples

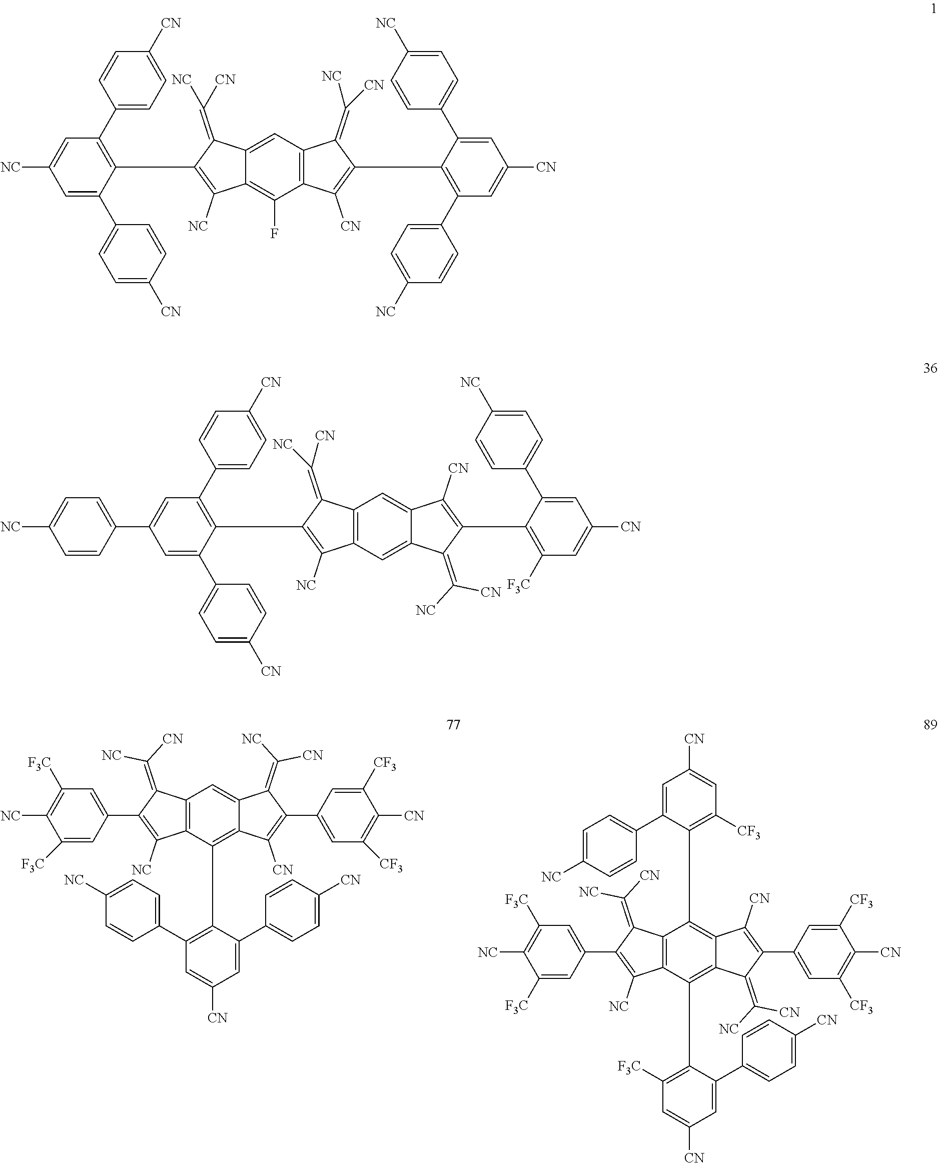

### Compounds of Comparative Examples

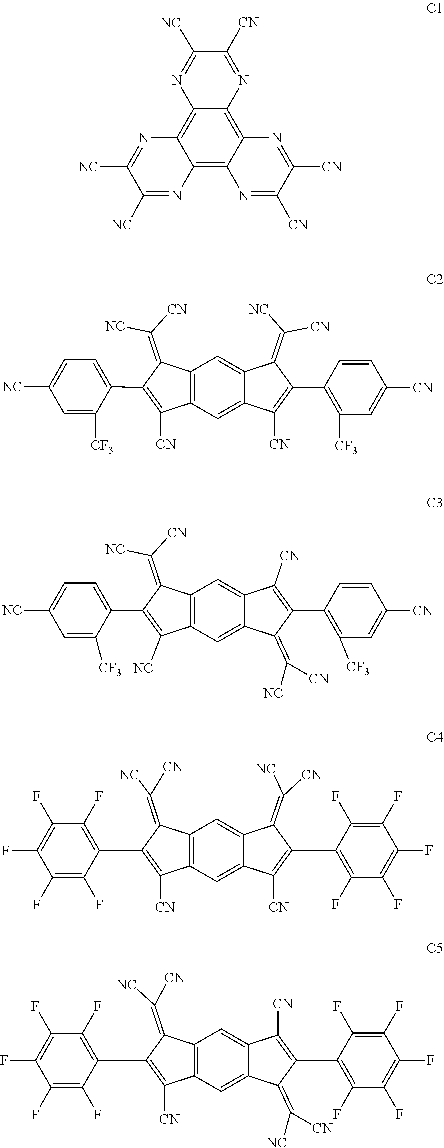 [SMILES: N#Cc1nc2c3nc(C#N)c(C#N)nc3c3nc(C#N)c(C#N)nc3c2nc1C#N ; N#CC(C#N)=C1C(c2ccc(C#N)cc2C(F)(F)F)=C(C#N)c2cc3c(cc21)C(=C(C#N)C#N)C(c1ccc(C#N)cc1C(F)(F)F)=C3C#N ; N#CC(C#N)=C1C(c2ccc(C#N)cc2C(F)(F)F)=C(C#N)c2cc3c(cc21)C(C#N)=C(c1ccc(C#N)cc1C(F)(F)F)C3=C(C#N)C#N ; N#CC(C#N)=C1C(c2c(F)c(F)c(F)c(F)c2F)=C(C#N)c2cc3c(cc21)C(=C(C#N)C#N)C(c1c(F)c(F)c(F)c(F)c1F)=C3C#N ; N#CC(C#N)=C1C(c2c(F)c(F)c(F)c(F)c2F)=C(C#N)c2cc3c(cc21)C(C#N)=C(c1c(F)c(F)c(F)c(F)c1F)C3=C(C#N)C#N]

The following compounds were used in the fabrication of the light-emitting device. Specifically, the commercially available products were used after sublimation purification.

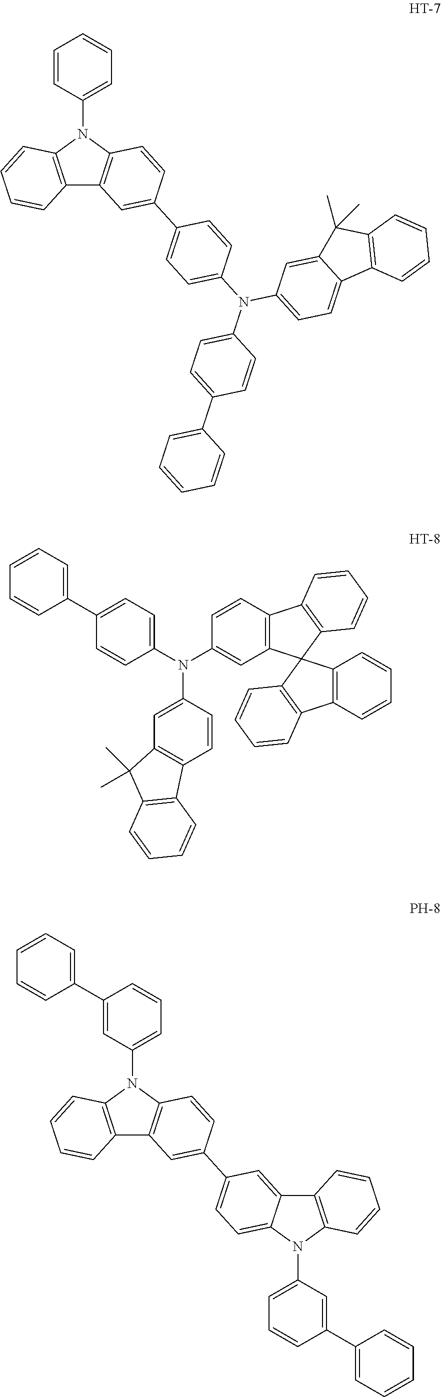 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2cccc(-n3c4ccccc4c4cc(-c5ccc6c(c5)c5ccccc5n6-c5cccc(-c6ccccc6)c5)ccc43)c2)cc1]

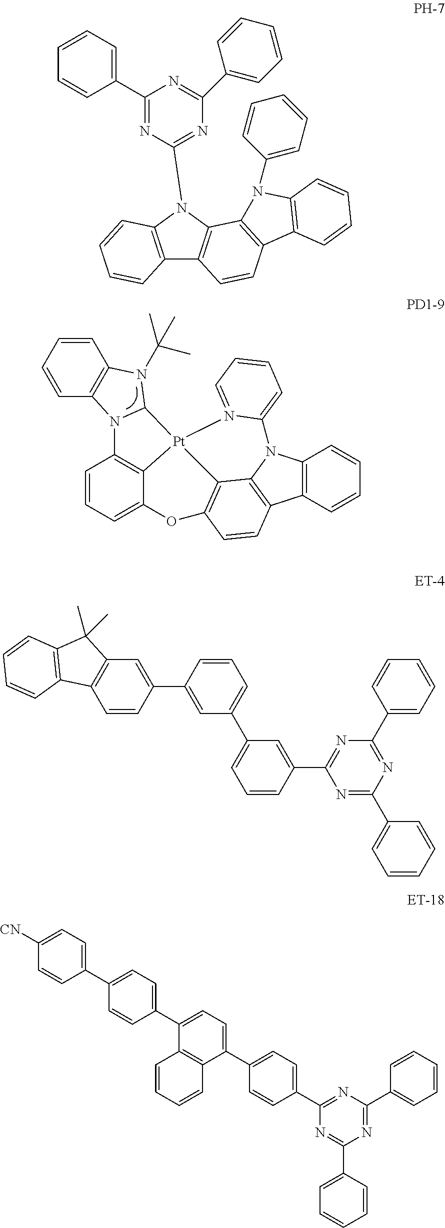

### Evaluation Example

### Evaluation Example 1: Evaluation on Properties of Polycyclic Compounds

Properties of the compounds of Examples and Comparative Examples were evaluated as follows, and the results are shown in Table 1 below.

### (1) HOMO Energy Level Evaluation

Using cyclic voltammetry (CV) (electrolyte: 0.1 M Bu₄NPF₆/solvent: DMF (dimethylforamide)/electrode: 3-electrode system (working electrode: GC (glassy carbon), reference electrode: Ag/AgCl, auxiliary electrode: Pt)), a potential (V)-current (A) graph of each compound was obtained. Thereafter, a HOMO energy level of each compound was calculated from an oxidation onset of the graph.

### (2) LUMO Energy Level Evaluation

Cyclic voltammetry (CV) (electrolyte: 0.1 M Bu₄NPF₆/solvent: DMF (dimethylforamide)/electrode: 3-electrode system (working electrode: GC, reference electrode: Ag/AgCl, auxiliary electrode: Pt)) was used to obtain a potential (V)-current (A) graph of each compound. Thereafter, a LUMO energy level of each compound was calculated from a reduction onset of the graph.

### (3) Hole Mobility and Electron Mobility Evaluation

A hole mobility and an electron mobility were evaluated using a space-charge-limited current (SCLC) method described in the literature, in particular “Hole mobility of N,N′-bis(naphtanlen-1-yl)-N,N′-bis(phenyl)benzidine investigated by using space-charge-limited currents”, Appl. Phys. Lett. 90, 203512 (2007).

### (4) Glass Transition Temperature Evaluation

A glass transition temperature (Tg) was measured by a DSC (differential scanning calorimetry) analysis. Specifically, 5 mg of a sample was heated from room temperature to 300° C. at a ramping rate of 10° C./min, cooled from 300° C. to 25° C., and then heated to 300° C. again. The glass transition temperature was obtained from an inflection point of the graph during the second heating.

TABLE 1

| | | | | hole | electron | glass transition |
|---|---|---|---|---|---|---|
| | | HOMO | LUMO | mobility | mobility | temperature |
| | compound | (eV) | (eV) | (cm²/Vs) | (cm²/Vs) | (° C.) |
| Comparative | C1 | −9.05 | −4.81 | 1.7E−03 | 5.0E−03 | 119 |
| Example 1 | | | | | | |
| Comparative | C2 | −7.91 | −5.22 | 5.4E−04 | 4.7E−03 | 118 |
| Example 2 | | | | | | |
| Comparative | C3 | −7.76 | −5.29 | 1.8E−04 | 3.3E−03 | 120 |
| Example 3 | | | | | | |
| Comparative | C4 | −7.76 | −5.25 | 1.2E−04 | 4.6E−03 | 114 |
| Example 4 | | | | | | |
| Comparative | C5 | −7.66 | −5.32 | 7.6E−05 | 5.1E−03 | 117 |
| Example 5 | | | | | | |
| Example 1 | 1 | −7.87 | −5.56 | 3.3E−04 | 1.6E−03 | 152 |
| Example 2 | 36 | −7.84 | −5.58 | 3.0E−04 | 1.3E−03 | 138 |
| Example 3 | 77 | −8.03 | −5.62 | 5.9E−04 | 8.8E−04 | 141 |
| Example 4 | 89 | −7.95 | −5.65 | 8.3E−04 | 1.3E−03 | 147 |

Referring to Table 1, in the polycyclic compounds according to Examples, the substituent capable of protecting the core was introduced, and deep LUMO energy, high hole mobility, low electron mobility, and high glass transition temperature were provided.

It can be predicted that the polycyclic compounds according to Examples may increase generation efficiency of excitons in the emission layer, and deterioration in a high-temperature process may be prevented by the high glass transition temperature.

The compounds according to Comparative Examples had shallow LUMO energy level, low hole mobility or high electron mobility, and low glass transition temperature. Evaluation Example 2. Performance evaluation of light-emitting device

Properties of the light-emitting devices fabricated as described above were measured at a current density of 10 mA/cm² using V7000 OLED IVL Test System, (Polaronix).

Specifically, a driving voltage at a luminance of 1000 cd/m² were measured using a source meter (Keithley MU 236), and a luminous efficacy (Cd/A) was measured using a luminance meter CS-2000 (Konica Minolta).

The light-emitting device was continuously driven at a current density of 10 mA/cm², and a time until a luminance dropped to 95% of an initial value was measured. A relative value with respect to the time measured in the light-emitting device using the compound of Comparative Example 2 was expressed as a life-span (T95) of each light-emitting device.

The results are shown in Table 2 below.

TABLE 2

| | | | luminous | |
|---|---|---|---|---|
| | | driving voltage | efficiency | relative life-span |
| | p-dopant | (V) | (cd/A/y) | (T95) |
| Comparative | C1 | 4.2 | 16.9 | 94% |
| Example 1 | | | | |
| Comparative | C2 | 3.8 | 17.4 | 100% |
| Example 2 | | | | |
| Comparative | C3 | 3.7 | 17.6 | 101% |
| Example 3 | | | | |
| Comparative | C4 | 3.7 | 17.5 | 100% |
| Example 4 | | | | |
| Comparative | C5 | 3.6 | 17.5 | 102% |
| Example 5 | | | | |
| Example 1 | 1 | 3.3 | 18.7 | 112% |
| Example 2 | 36 | 3.4 | 18.4 | 109% |
| Example 3 | 77 | 3.3 | 18.6 | 112% |
| Example 4 | 89 | 3.2 | 18.8 | 114% |

Referring to Table 2, in the light-emitting devices including the polycyclic compounds according to Examples, the driving voltage was lowered, and the luminous efficiency and the relative life-span were improved. An interaction between the polycyclic compounds of Examples and surrounding compounds in the hole injection layer was suppressed or prevented, thereby improving hole injection performance.

In the light-emitting devices including the compounds according to Comparative Examples, the driving voltage was increased, and the luminous efficiency and relative life-span were decreased.

What is claimed is:

1. A polycyclic compound represented by Chemical Formula 1 or Chemical Formula 2: 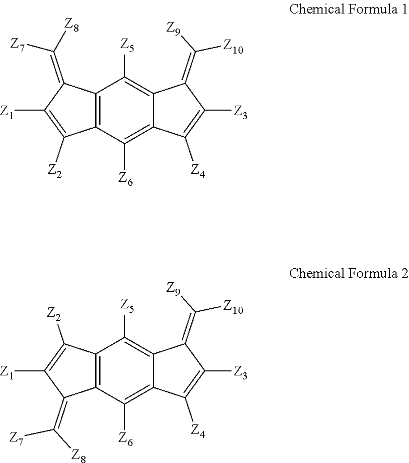
wherein, in Chemical Formulae 1 and 2, Z₁ to Z₁₀ are each independently hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ arylalkyl group, a substituted or unsubstituted C₇-C₆₀ alkyl aryl group, a substituted or unsubstituted C₂-C₆₀ heteroaryl group, a substituted or unsubstituted C₂-C₆₀ alkyl heteroaryl group, a substituted or unsubstituted C₃-C₆₀ heteroarylalkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, or a substituted or unsubstituted silyl group, and two or more adjacent groups among Z₁ to Z₁₀ are optionally combined to form a saturated ring or an unsaturated ring,
at least one of Z₁ to Z₁₀ is a substituted or unsubstituted biphenyl group, a substituted or unsubstituted terphenyl group, a substituted or unsubstituted dibenzofuran group, a substituted or unsubstituted dibenzothiophene group, or a substituted or unsubstituted carbazole group, and at least one of a remainder of Z₁ to Z₁₀ is an electron withdrawing group or a group substituted with an electron withdrawing group, and
the electron-withdrawing group is a group having a para Hammett substituent constant (σp) of a Hammett rule greater than 0.

2. The polycyclic compound of claim 1, wherein at least one of Z₁, Z₃, Z₅ and Z₆ is groups represented by Chemical Formulae 3-1 to 3-4: 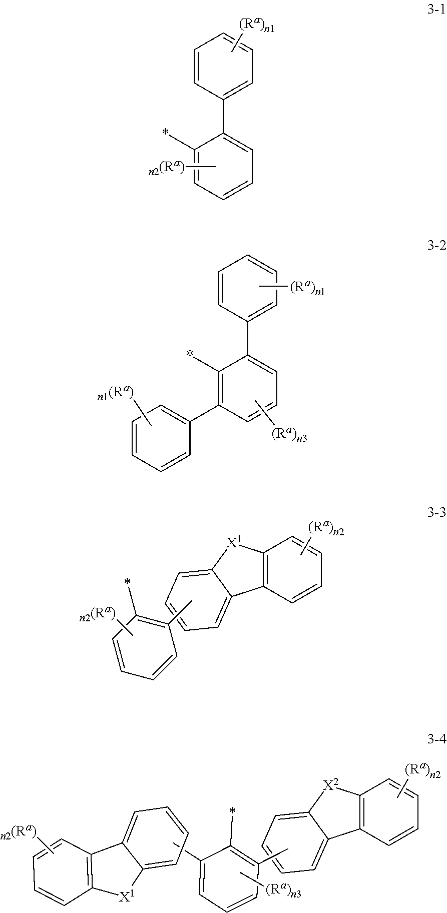
wherein, in Chemical Formulae 3-1 to 3-4, R^{a} is hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₃₀ alkyl group, a substituted or unsubstituted C₂-C₃₀ alkenyl group, a substituted or unsubstituted C₂-C₃₀ alkynyl group, a substituted or unsubstituted C₁-C₃₀ alkoxy group, a C₁-C₃₀ alkylthio group, a substituted or unsubstituted C₃-C₃₀ cycloalkyl group, a substituted or unsubstituted C₅-C₃₀ cycloalkenyl group, a substituted or unsubstituted C₁-C₃₀ heterocycloalkyl group, a substituted or unsubstituted C₁-C₃₀ heterocycloalkenyl group, a substituted or unsubstituted C₆-C₃₀ aryl group, a substituted or unsubstituted C₇-C₃₀ arylalkyl group, a substituted or unsubstituted C₇-C₃₀ alkyl aryl group, a substituted or unsubstituted C₂-C₃₀ heteroaryl group, a substituted or unsubstituted C₂-C₃₀ alkyl heteroaryl group, a substituted or unsubstituted C₃-C₃₀ heteroarylalkyl group, a substituted or unsubstituted C₆-C₃₀ aryloxy group, or a substituted or unsubstituted C₆-C₃₀ arylthio group,
when a plurality of R^{a} is included, the plurality of R^{a} is the same as or different from each other, and two or more adjacent groups among the plurality of R^{a} are optionally combined to form a saturated ring or an unsaturated ring,
n1 is an integer from 0 to 5, n2 is an integer from 0 to 4, and n3 is an integer from 0 to 3,
X¹ and X² are each independently CR₁R₂, NR₃, O, S, or Se,
R₁ to R₃ are each independently hydrogen, deuterium, —CD₃, —CD₂H, —CDH₂, —CF₃, —CF₂H, —CFH₂, a substituted or unsubstituted C₁-C₃₀ alkyl group, a substituted or unsubstituted C₆-C₃₀ aryl group, a substituted or unsubstituted C₇-C₃₀ arylalkyl group, a substituted or unsubstituted C₇-C₃₀ alkyl aryl group, a substituted or unsubstituted C₂-C₃₀ heteroaryl group, a substituted or unsubstituted C₂-C₃₀ alkyl heteroaryl group, or a substituted or unsubstituted C₃-C₃₀ heteroarylalkyl group, and
*- represents a bonding position.

3. The polycyclic compound of claim 2, wherein, Z₁ and Z₃ are each independently a group represented by Chemical Formulae 3-1 to 3-4, or Z₅ and Z₆ are each independently Chemical Formulae 3-1 to 3-4.

4. The polycyclic compound of claim 1, wherein the electron withdrawing group is from —F, —Cl, —Br, —I, —SF₅, —CF₃, —CN, —SCN, —SOCH₃, —SOCH₂CH₃, —SCH(CH₃)₂, —NO₂, and a Tr electron-depleted nitrogen-containing C₃-C₃₀ cyclic group.

5. The polycyclic compound of claim 4, wherein the electron withdrawing group is —F, —Cl, —Br, —I, —SF₅, —CF₃, —CN, —SCN, —SOCH₃, —SOCH₂CH₃, —SCH(CH₃)₂, —NO₂, a substituted or unsubstituted triazine group, a substituted or unsubstituted thiazole group, a substituted or unsubstituted benzothiazole group, a substituted or unsubstituted pyrazine group, a substituted or unsubstituted pyridine group, a substituted or unsubstituted pyridazine group, a substituted or unsubstituted pyrimidine group, a substituted or unsubstituted naphthyridine group, a substituted or unsubstituted phthalazine group, a substituted or unsubstituted quinoline group, a substituted or unsubstituted quinazoline group, a substituted or unsubstituted benzocinnoline group, a substituted or unsubstituted phenanthroline group, or a substituted or unsubstituted acridine group.

6. The polycyclic compound of claim 1, wherein the polycyclic compound is represented by any one of Chemical Formulae 1-1 to 1-10: 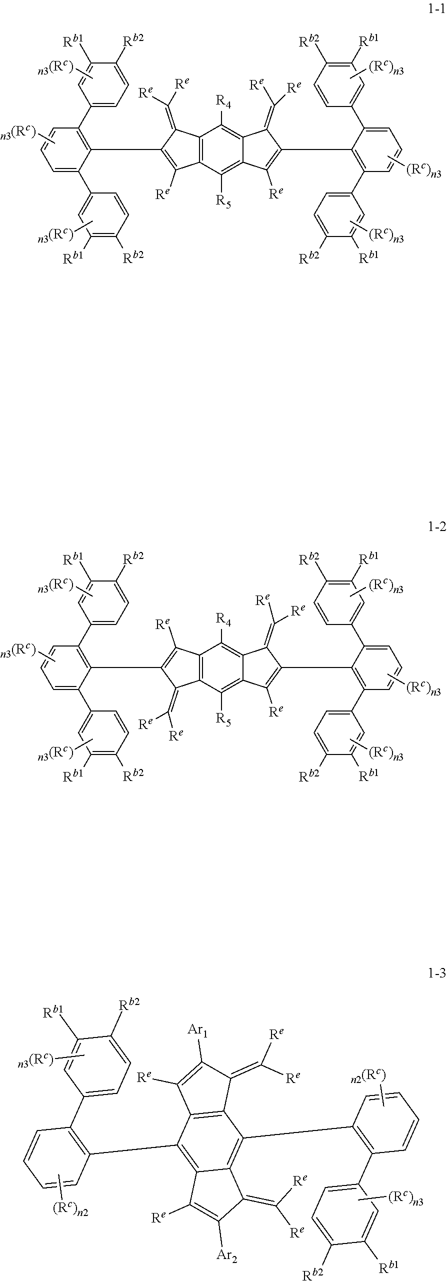 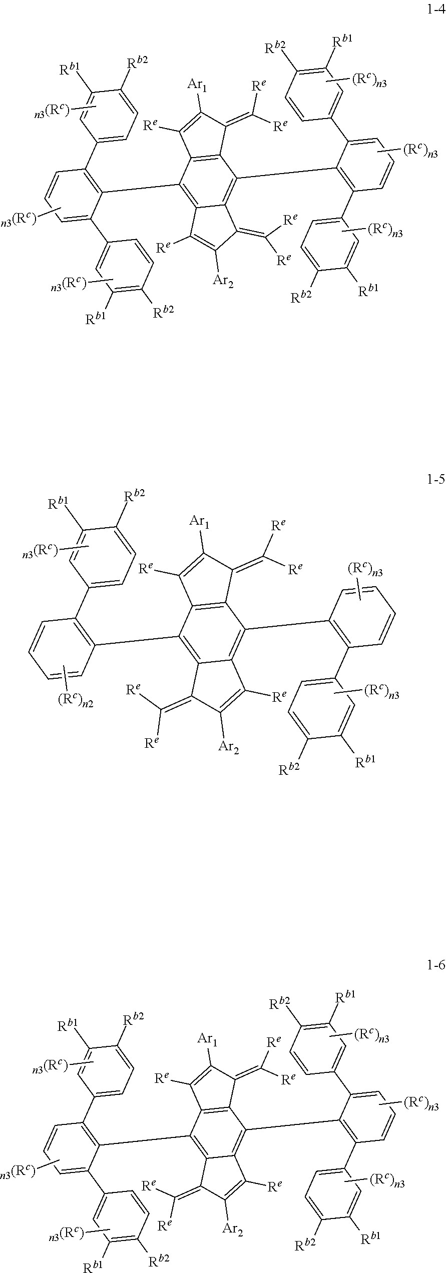 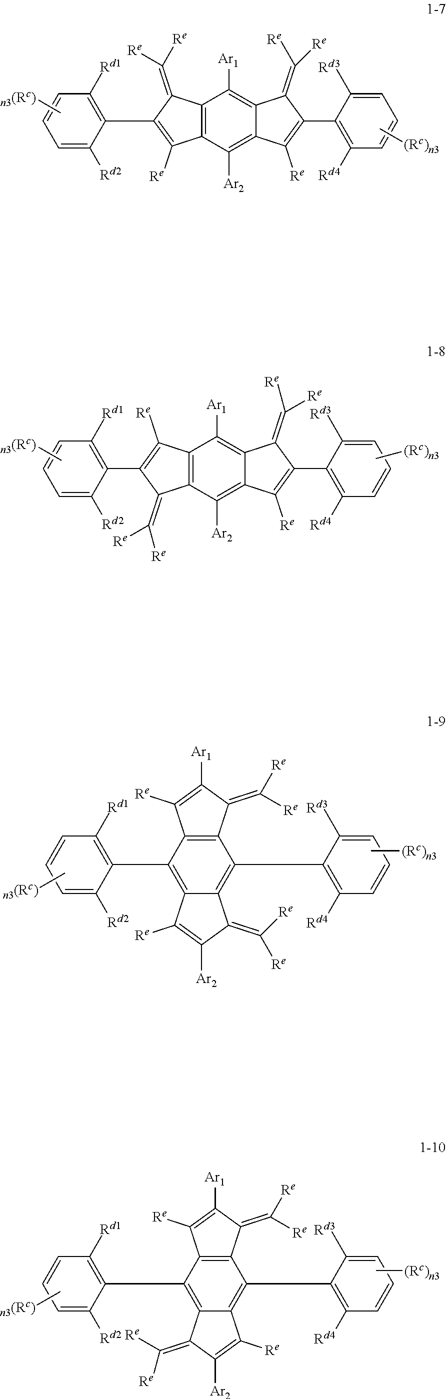
wherein, in Chemical Formulae 1-1 to 1-10, n2 is an integer from 0 to 4, and n3 is an integer from 0 to 3,
R^{c} is hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₃₀ alkyl group, a substituted or unsubstituted C₃-C₃₀ cycloalkyl group, a substituted or unsubstituted C₁-C₃₀ heterocycloalkyl group, a substituted or unsubstituted C₆-C₃₀ aryl group, a substituted or unsubstituted C₇-C₃₀ arylalkyl group, a substituted or unsubstituted C₇-C₃₀ alkyl aryl group, a substituted or unsubstituted C₂-C₃₀ heteroaryl group, a substituted or unsubstituted C₂-C₆₀ alkyl heteroaryl group, or a substituted or unsubstituted C₃-C₃₀ heteroarylalkyl group,
when a plurality of R^{c} is included, two or more adjacent groups among the plurality of R^{c} are optionally combined to form a saturated ring or an unsaturated ring,
a plurality of each of R^{b1} and R^{b2} are the same as or different from each other, and are each independently hydrogen, deuterium, —CN, —F, —CD₃, —CD₂H, —CDH₂, —CF₃, —CF₂H, —CFH₂, a substituted or unsubstituted C₆-C₃₀ aryl group, or a substituted or unsubstituted C₂-C₃₀ heteroaryl group,
Ar₁ and Ar₂ are each independently a substituted or unsubstituted C₆-C₃₀ aryl group, or a substituted or unsubstituted C₂-C₃₀ heteroaryl group,
two or more R^{c} are the same as or different from each other, and are each independently the electron withdrawing group,
R₄ and R₅ are each independently hydrogen, deuterium, —CD₃, —CD₂H, —CDH₂, —CF₃, —CF₂H, —CFH₂, a substituted or unsubstituted C₁-C₃₀ alkyl group, or the electron withdrawing group, and
R^{d1} to R^{d4} are each independently the electron withdrawing group, a substituted or unsubstituted dibenzofuran group, a substituted or unsubstituted dibenzothiophene group, or a substituted or unsubstituted carbazole group, and two or more of R^{d1} to R^{d4} are each independently a substituted or unsubstituted dibenzofuran group, a substituted or unsubstituted dibenzothiophene group, or a substituted or unsubstituted carbazole group.

7. The polycyclic compound of claim 6, wherein, in Chemical Formulae 1-1 to 1-6, two or more R^{b1} are each independently a substituted or unsubstituted C₆-C₃₀ aryl group, or a substituted or unsubstituted C₂-C₃₀ heteroaryl group, and two or more R^{b2} are each independently hydrogen, deuterium, —CN, —F, —CD₃, —CD₂H, —CDH₂, —CF₃, —CF₂H, or —CFH₂.

8. The polycyclic compound of claim 6, wherein, in Chemical Formulae 1-1 and 1-7, R₄ is the electron withdrawing group, and R₅ is hydrogen, deuterium, —CD₃, —CD₂H, —CDH₂, or a substituted or unsubstituted C₁-C₃₀ alkyl group.

9. The polycyclic compound of claim 6, wherein, in Chemical Formulae 1-7 to 1-10, at least one of R^{d1} and R^{d2}, and at least one of R^{d3} and R^{d4} are each independently a substituted or unsubstituted dibenzofuran group, a substituted or unsubstituted dibenzothiophene group, or a substituted or unsubstituted carbazole group.

10. The polycyclic compound of claim 6, wherein a degree of deuteration of the polycyclic compound according to Equation 1 is in a range from 10% to 100%:
Degree of Deuteration (%)=(the number of deuterium atoms)/(the number of deuterium atoms+the number of hydrogen atoms)×100 [Equation 1]
wherein, in Equation 1, the number of deuterium atoms and the number of hydrogen atoms refer to the numbers of deuterium and hydrogen atoms, respectively, included in the polycyclic compound.

11. The polycyclic compound of claim 1, wherein the polycyclic compound has a hole mobility in a range from 2.5×10⁻⁴ cm²/Vs to 1.0×10⁻³ cm²/Vs.

12. The polycyclic compound of claim 1, wherein the polycyclic compound has an electron mobility in a range from 8.0×10⁻⁴ cm²/Vs to 2.5×10⁻³ cm²/Vs.

13. The polycyclic compound of claim 1, wherein the polycyclic compound has a glass transition temperature of 125° C. or more.

14. The polycyclic compound of claim 1, wherein the polycyclic compound has a LUMO energy level in a range from −5.0 eV to −6.0 eV.

15. A light-emitting device, comprising:
a first electrode;
a second electrode; and
an intermediate disposed between the first electrode and the second electrode, the intermediate layer comprising at least one light-emitting structure that comprises a hole transfer region, an emission layer and an electron transfer region,
wherein the hole transfer region includes a polycyclic compound represented by Chemical Formula 1 or Chemical Formula 2: 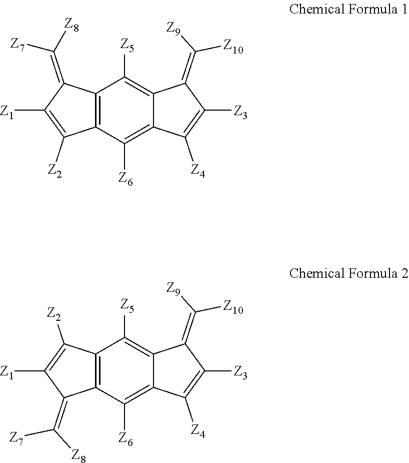
wherein, in Chemical Formulae 1 and 2, Z₁ to Z₁₀ are each independently hydrogen, deuterium, —CN, —F, —Cl, —Br, —I, —SF₅, —CD₃, —CD₂H, —CDH₂, —CF₃, —CF₂H, —CFH₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₁-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₇-C₆₀ arylalkyl group, a substituted or unsubstituted C₇-C₆₀ alkyl aryl group, a substituted or unsubstituted C₂-C₆₀ heteroaryl group, a substituted or unsubstituted C₂-C₆₀ alkyl heteroaryl group, a substituted or unsubstituted C₃-C₆₀ heteroarylalkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, or a substituted or unsubstituted silyl group, and two or more adjacent groups among Z₁ to Z₁₀ are optionally combined to form a saturated ring or an unsaturated ring,
at least one of Z₁ to Z₁₀ is from a substituted or unsubstituted biphenyl group, a substituted or unsubstituted terphenyl group, a substituted or unsubstituted dibenzofuran group, a substituted or unsubstituted dibenzothiophene group, or a substituted or unsubstituted carbazole group, and at least one of a remainder of Z₁ to Z₁₀ is an electron withdrawing group or a group substituted with an electron withdrawing group, and
the electron-withdrawing group is a group having a para Hammett substituent constant (σp) of a Hammett rule greater than 0.

16. The light-emitting device of claim 15, wherein the intermediate layer comprises two or more of the light-emitting structures, and at least one of the hole transfer region includes the polycyclic compound.

17. The light-emitting device of claim 15, wherein the hole transfer region comprises a hole injection layer, and the hole injection layer includes the polycyclic compound.

18. The light-emitting device of claim 17, wherein the polycyclic compound is included in an amount of 1 wt % to 40 wt % based on a total weight of the hole injection layer.

19. An electronic device comprising the light-emitting device of claim 15.

20. The electronic device of claim 19, wherein the electronic device is one of a flat panel display, a curved display, a computer monitor, a medical monitor, a television, a billboard, a light for indoor or outdoor lighting, a signal lighting, a head-up display, a full or partial transparent display, a flexible display, a rollable display, a foldable display, a stretchable display, a laser printer, a phone, a mobile phone, a tablet, a phablet, a personal information terminal (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.
